(19) United States
(12) Patent Application Publication
(10) Pub. No.: US 2025/0164884 A1
(43) Pub. Date: May 22, 2025

(54) METHOD FOR PRODUCING ACTIVE RAY-SENSITIVE OR RADIATION-SENSITIVE RESIN COMPOSITION, METHOD FOR PRODUCING ONIUM SALT COMPOUND FOR ACTIVE RAY-SENSITIVE OR RADIATION-SENSITIVE RESIN COMPOSITION, PATTERN FORMING METHOD, METHOD FOR PRODUCING ELECTRONIC DEVICE, AND ONIUM SALT COMPOSITION

(71) Applicant: FUJIFILM CORPORATION, Tokyo (JP)

(72) Inventors: Akihiro Kaneko, Shizuoka (JP); Akiyoshi Goto, Shizuoka (JP)

(21) Appl. No.: 19/035,324

(22) Filed: Jan. 23, 2025

Related U.S. Application Data
(63) Continuation of application No. PCT/JP2023/027402, filed on Jul. 26, 2023.

(30) Foreign Application Priority Data
Feb. 22, 2022 (JP) 2023-026626
Jul. 29, 2022 (JP) 2022-122353
Aug. 23, 2022 (JP) 2022-132859

(51) Int. Cl. G03F 7/06; G03F 7/004

(52) U.S. Cl. CPC G03F 7/066; G03F 7/0045

(57) ABSTRACT

The present invention provides a method for producing an actinic ray-sensitive or radiation-sensitive resin composition, including (X) mixing a specific onium salt compound (1) with a specific salt compound (2) in a non-aqueous solvent (S) to obtain a specific onium salt compound (3), (Y) obtaining a specific onium salt compound (A) from the onium salt compound (3), and (Z) mixing the onium salt compound (A) with a resin (B) whose solubility in a developer changes due to action of acid; a method for producing an onium salt compound (A) for an actinic ray-sensitive or radiation-sensitive resin composition, including (X) and (Y); a pattern forming method; and a method for manufacturing an electronic device.

### CROSS REFERENCE TO RELATED APPLICATION

This is a continuation of International Application No. PCT/JP2023/027402 filed on Jul. 26, 2023, and claims priorities from Japanese Patent Application No. 2022-122353 filed on Jul. 29, 2022, Japanese Patent Application No. 2022-132859 filed on Aug. 23, 2022, and Japanese Patent Application No. 2023-026626 filed on Feb. 22, 2023, the entire disclosures of which are incorporated herein by reference.

### BACKGROUND OF THE INVENTION

### 1. Field of the Invention

The present invention relates to a method for producing an actinic ray-sensitive or radiation-sensitive resin composition, a method for producing an onium salt compound for an actinic ray-sensitive or radiation-sensitive resin composition, a pattern forming method, a method for manufacturing an electronic device, and an onium salt composition. More specifically, the present invention relates to a method for producing an actinic ray-sensitive or radiation-sensitive resin composition, which can be suitably used for an ultra-microlithography process applicable to a manufacturing process of an ultra large scale integration (LSI) and a high-capacity microchip, a mold creation process for a nanoimprint, a manufacturing process of high-density information recording medium, and other photofabrication processes, a method for producing an onium salt compound, which can be suitably used for the actinic ray-sensitive or radiation-sensitive resin composition, a pattern forming method, a method for manufacturing an electronic device, and an onium salt composition.

### 2. Description of the Related Art

In processes for manufacturing semiconductor devices such as an integrated circuit (IC) and a large scale integration circuit (LSI), microfabrication by lithography using a resist composition has been performed in the related art. In recent years, along with high integration of integrated circuits, a formation of ultrafine patterns in a submicron region or quarter micron region has been required. Along with this, there is a tendency for an exposure wavelength to be shortened, such as from g-rays to i-rays and to KrF excimer laser light, and at present, an exposure machine using, as a light source, an ArF excimer laser having a wavelength of 193 nm has been developed. In addition, as a technique for further enhancing resolving power, development of a so-called immersion method in which a liquid having a high refractive index (hereinafter, also referred to as “immersion liquid”) is filled between a projection lens and a sample has been progressed in the related art.

Furthermore, at present, the development of lithography using electron beams (EB), X-rays, extreme ultraviolet rays (EUV), or the like in addition to excimer laser light is also in progress. In association with this, various resist-sensitive actinic ray-sensitive or radiation-sensitive resin compositions which are effectively sensitive to actinic rays or radiation have been developed.

Various onium salt compounds and manufacturing methods thereof, which are used as salts of acids used in actinic ray-sensitive or radiation-sensitive resin compositions, are known. For example, JP2014-97969A specifically discloses a method for producing an onium salt compound useful as an acid generator component of a photoresist composition, the method including a step of obtaining a synthetic intermediate by subjecting an onium salt compound having a triflate anion (CF₃SO₃⁻, hereinafter also referred to as TfO⁻) and an iodide salt to an ion exchange reaction using a dichloromethane/aqueous solvent.

### SUMMARY OF THE INVENTION

In recent years, miniaturization of a pattern formed using EUV or an electron beam has been promoted, and further improvement in the performance of a resist composition, such as line width roughness (LWR) performance, is required. The LWR performance refers to performance of reducing the LWR of a pattern.

However, it has been found that the resist composition using the onium salt compound produced by the method for producing described in JP2014-97969A has insufficient LWR performance.

An object of the present invention is to provide a method for producing an actinic ray-sensitive or radiation-sensitive resin composition having excellent LWR performance in a pattern formation (for example, a line-and-space pattern having a line width of 25 nm or less or a hole pattern having a hole diameter of 25 nm or less), a pattern forming method including the method for producing, and a method for manufacturing an electronic device.

In addition, another object of the present invention is to provide a method for producing an onium salt compound, which can be suitably used for the actinic ray-sensitive or radiation-sensitive resin composition, and an onium salt composition.

The present inventors have found that the above-described objects can be achieved by the following configurations.

[1]

A method for producing an actinic ray-sensitive or radiation-sensitive resin composition, the method including:
mixing an onium salt compound (1) represented by Formula (1A) or Formula (1B) with a salt compound (2) represented by Formula (2) in a non-aqueous solvent (S) to obtain an onium salt compound (3) represented by Formula (3A) or Formula (3B);
obtaining an onium salt compound (A) represented by Formula (4A) or Formula (4B) from the onium salt compound (3); and
mixing the onium salt compound (A) with a resin (B) whose solubility in a developer changes due to action of acid.

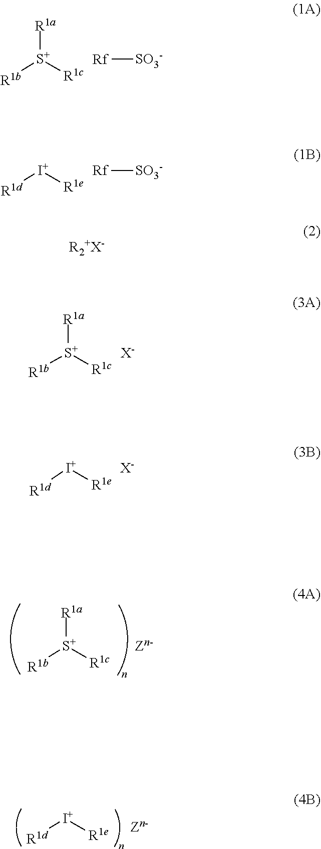

In Formulae (1A), (3A), and (4A), R^{1a}, R^{1b}, and R^{1a} each independently represent an alkyl group, a cycloalkyl group, an aryl group, or *—W₁—C(═O)Ar¹. Two of R^{1a}, R^{1b}, and R^{1c} may be bonded to each other to form a ring. *represents a linkage site to S⁺, W₁ represents a single bond or an alkylene group, and Ar¹ represents an aryl group.

In Formulae (1B), (3B), and (4B), R^{1d} and R^{1e} each independently represent an alkyl group, a cycloalkyl group, or an aryl group.

In Formulae (1A) and (1B), Rf represents an alkyl group or an aryl group which includes one or more fluorine atoms.

In Formula (2), R₂⁺ represents an organic cation having no polymer structure. X⁻ represents Cl⁻ or Br⁻.

In Formulae (3A) and (3B), X⁻ represents Cl⁻ or Br⁻.

In Formulae (4A) and (4B), Zⁿ⁻ represents an n-valent organic anion. n represents an integer of 1 or more.

[2]

The method for producing an actinic ray-sensitive or radiation-sensitive resin composition as described in [1], in which R^{1a}, R^{1b}, and R^{1c} in Formula (1A), Formula (3A), and Formula (4A) each independently represent an alkyl group which does not include a halogen atom, a cycloalkyl group, an aryl group, or *—W₁—C(═O)Ar¹.

However, two of R^{1a}, R^{1b}, and R^{1c} may be bonded to each other to form a ring. *represents a linkage site to S⁺, W₁ represents a single bond or an alkylene group, and Ar¹ represents an aryl group.

[3]

The method for producing an actinic ray-sensitive or radiation-sensitive resin composition as described in [1] or [2], in which R₂⁺ in Formula (2) is an organic cation represented by Formula (2B).

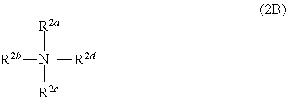

In Formula (2B), R^{2a} to R^{2d} each independently represent an alkyl group, a cycloalkyl group, or an aryl group.

[4]

The method for producing an actinic ray-sensitive or radiation-sensitive resin composition as described in any one of [1] to [3], in which the non-aqueous solvent (S) contains at least one of an ester-based solvent or an ether-based solvent.

[5]

The method for producing an actinic ray-sensitive or radiation-sensitive resin composition as described in any one of [1] to [4], in which R^{1a}, R^{1b}, and R^{1c} in Formulae (1A), (3A), and (4A) are aryl groups.

However, two of R^{1a}, R^{1b}, and R^{1c} may be bonded to each other to form a ring.

[6]

A method for producing an onium salt compound (A) for an actinic ray-sensitive or radiation-sensitive resin composition, the method including:
mixing an onium salt compound (1) represented by Formula (1A) or Formula (1B) with a salt compound (2) represented by Formula (2) in a non-aqueous solvent (S) to obtain an onium salt compound (3) represented by Formula (3A) or Formula (3B); and
obtaining an onium salt compound (A) represented by Formula (4A) or Formula (4B) from the onium salt compound (3).

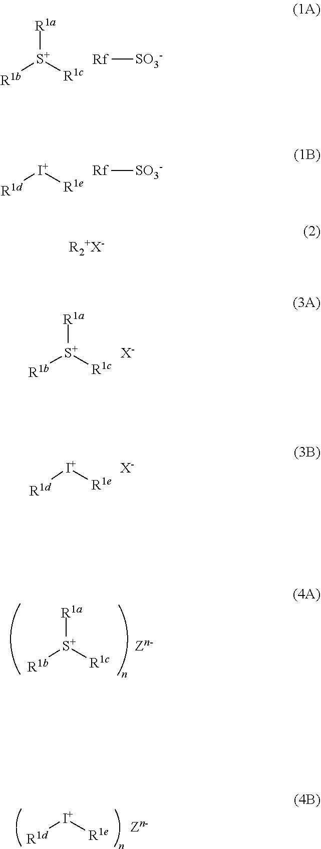

In Formulae (1A), (3A), and (4A), R^{1a}, R^{1b}, and R^{1c} each independently represent an alkyl group, a cycloalkyl group, an aryl group, or *—W₁—C(═O)Ar¹. Two of R^{1a}, R^{1b}, and R^{1c} may be bonded to each other to form a ring. *represents a linkage site to S⁺, W₁ represents a single bond or an alkylene group, and Ar¹ represents an aryl group.

In Formulae (1B), (3B), and (4B), R^{1d} and R^{1e} each independently represent an alkyl group, a cycloalkyl group, or an aryl group.

In Formulae (1A) and (1B), Rf represents an alkyl group or an aryl group which includes one or more fluorine atoms.

In Formula (2), R₂⁺ represents an organic cation having no polymer structure. X⁻ represents Cl⁻ or Br⁻.

In Formulae (3A) and (3B), X⁻ represents Cl⁻ or Br⁻.

In Formulae (4A) and (4B), Zⁿ⁻ represents an n-valent organic anion. n represents an integer of 1 or more.

[7]

The method for producing an onium salt compound (A) according to [6], in which R^{1a}, R^{1b}, and R^{1c} in Formula (1A), Formula (3A), and Formula (4A) each independently represent an alkyl group which does not include a halogen atom, a cycloalkyl group, an aryl group, or *—W₁—C(═O)Ar¹.

However, two of R^{1a}, R^{1b}, and R^{1c} may be bonded to each other to form a ring. *represents a linkage site to S⁺, W₁ represents a single bond or an alkylene group, and Ar¹ represents an aryl group.

[8]

The method for producing an onium salt compound (A) according to [6] or [7], in which R₂⁺ in Formula (2) is an organic cation represented by Formula (2B).

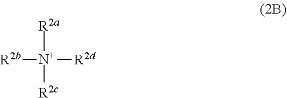

In Formula (2B), R^{2a} to R^{2d} each independently represent an alkyl group, a cycloalkyl group, or an aryl group.

[9]

The method for producing an onium salt compound (A) according to any one of [6] to [8], in which the non-aqueous solvent (S) contains at least one of an ester-based solvent or an ether-based solvent.

[10]

The method for producing an onium salt compound (A) according to any one of [6] to [9], in which R^{1a}, R^{1b}, and R^{1c} in Formulae (1A), (3A), and (4A) are aryl groups.

However, two of R^{1a}, R^{1b}, and R^{1c} may be bonded to each other to form a ring.

[11]

An onium salt composition including an onium salt compound, including: 0.001 mol % to 3 mol % of an organic cation represented by Formula (2A) with respect to 1 mol of the onium salt compound represented by Formula (4A) or Formula (4B).

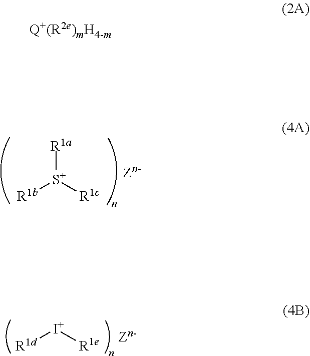

In Formula (2A), Q represents an N atom or a P atom, m represents an integer of 1 to 4, R^{2e} represents an alkyl group, a cycloalkyl group, or an aryl group, and a plurality of R^{2e}'s may be the same as or different from each other. R^{2e}'s adjacent to each other may form a ring.

In Formula (4A), R^{1a}, R^{1b}, and R^{1c} each independently represent an alkyl group, a cycloalkyl group, an aryl group, or *—W₁—C(═O)Ar¹. Two of R^{1a}, R^{1b}, and R^{1c} may be bonded to each other to form a ring. *represents a linkage site to S⁺, W₁ represents a single bond or an alkylene group, and Ar¹ represents an aryl group.

In Formula (4B), R^{1d} and R^{1e} each independently represent an alkyl group, a cycloalkyl group, or an aryl group.

In Formulae (4A) and (4B), Zⁿ⁻ represents an n-valent organic anion. n represents an integer of 1 or more.

[12]

The onium salt composition according to [11], in which the organic cation represented by Formula (2A) is represented by Formula (2B).

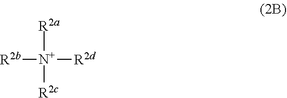

In Formula (2B), R^{2a} to R^{2d} each independently represent an alkyl group, a cycloalkyl group, or an aryl group.

[13]

The onium salt composition according to [11] or [12], in which R^{1a}, R^{1b}, and R^{1c} in Formula (4A) are aryl groups.

[14]

A pattern forming method including:
obtaining an actinic ray-sensitive or radiation-sensitive resin composition by the method for producing an actinic ray-sensitive or radiation-sensitive resin composition according to any one of [1] to [5];
forming an actinic ray-sensitive or radiation-sensitive film on a substrate by using the actinic ray-sensitive or radiation-sensitive resin composition; exposing the actinic ray-sensitive or radiation-sensitive film; and
developing the exposed actinic ray-sensitive or radiation-sensitive film using a developer to form a pattern.
   [15]

A method for manufacturing an electronic device, including:
the pattern forming method according to [14].

According to the present invention, it is possible to provide a method for producing an actinic ray-sensitive or radiation-sensitive resin composition having excellent LWR performance, a pattern forming method including the method for producing, and a method for manufacturing an electronic device.

In addition, according to the present invention, it is possible to provide a method for producing an onium salt compound, which can be suitably used for the actinic ray-sensitive or radiation-sensitive resin composition, and an onium salt composition.

### DESCRIPTION OF THE PREFERRED EMBODIMENTS

Hereinafter, the present invention will be described in detail.

Description of configuration requirements described below may be made on the basis of representative embodiments of the present invention in some cases, but the present invention is not limited to such embodiments.

“Actinic rays” or “radiation” in the present specification means, for example, a bright line spectrum of a mercury lamp, far ultraviolet rays typified by an excimer laser, extreme ultraviolet rays (EUV), X-rays, soft X-rays, electron beams (EB), or the like.

“Light” in the present specification means actinic ray or radiation.

Unless otherwise specified, “exposure” in the present specification encompasses not only exposure by a bright line spectrum of a mercury lamp, far ultraviolet rays typified by an excimer laser, extreme ultraviolet rays, X-rays, EUV, or the like, but also lithography by particle beams such as electron beams and ion beams.

In the present specification, a numerical range expressed using “to” is used in a meaning of a range that includes the preceding and succeeding numerical values of “to” as the lower limit value and the upper limit value, respectively.

In the present specification, (meth)acrylate represents at least one of acrylate or methacrylate. In addition, (meth)acrylic acid represents at least one of acrylic acid or methacrylic acid.

In the present specification, the weight-average molecular weight (Mw), the number-average molecular weight (Mn), and the dispersity (also referred to as a molecular weight distribution) (Mw/Mn) of a resin are each defined as a value expressed in terms of polystyrene by means of gel permeation chromatography (GPC) measurement (solvent: tetrahydrofuran, flow amount (amount of a sample injected): 10 μL, columns: TSK gel Multipore HXL-M manufactured by Tosoh Corporation, column temperature: 40° C., flow rate: 1.0 mL/min, detector: differential refractive index detector) using a GPC apparatus (HLC-8120 GPC manufactured by Tosoh Corporation).

In notations for a group (atomic group) in the present specification, in a case where the group is cited without specifying that it is substituted or unsubstituted, the group includes both a group having no substituent and a group having a substituent as long as it does not impair the spirit of the present invention. For example, an “alkyl group” includes not only an alkyl group having no substituent (unsubstituted alkyl group), but also an alkyl group having a substituent (substituted alkyl group). In addition, an “organic group” in the present specification refers to a group including at least one carbon atom.

A substituent is preferably a monovalent substituent unless otherwise specified. Examples of the substituent include a monovalent non-metal atomic group from which a hydrogen atom has been excluded, and the substituent can be selected from the following substituent T, for example.

### (Substituent T)

Examples of the substituent T include a halogen atom such as a fluorine atom, a chlorine atom, a bromine atom, and an iodine atom; an alkoxy group such as a methoxy group, an ethoxy group, and a tert-butoxy group; a cycloalkyloxy group; an aryloxy group such as a phenoxy group and a p-tolyloxy group; an alkoxycarbonyl group such as a methoxycarbonyl group and a butoxycarbonyl group; a cycloalkyloxycarbonyl group; an aryloxycarbonyl group such as a phenoxycarbonyl group; an acyloxy group such as an acetoxy group, a propionyloxy group, and a benzoyloxy group; an acyl group such as an acetyl group, a benzoyl group, an isobutyryl group, an acryloyl group, a methacryloyl group, and a methoxalyl group; a sulfanyl group; an alkylsulfanyl group such as a methylsulfanyl group and a tert-butylsulfanyl group; an arylsulfanyl group such as a phenylsulfanyl group and a p-tolylsulfanyl group; an alkyl group; an alkenyl group; a cycloalkyl group; an aryl group; an aromatic heterocyclic group; a hydroxy group; a carboxyl group; a formyl group; a sulfo group; a cyano group; an alkylaminocarbonyl group; an arylaminocarbonyl group; a sulfonamide group; a silyl group; an amino group; and a carbamoyl group. In addition, in a case where these substituents can further have one or more substituents, a group having one or more substituents selected from the above-described substituents as the further substituent (for example, a monoalkylamino group, a dialkylamino group, an arylamino group, or a trifluoromethyl group) is also included in the examples of the substituent T.

A bonding direction of divalent groups cited in the present specification is not limited unless otherwise specified. For example, in a case where Y in a compound represented by Formula “X—Y—Z” is —COO—, Y may be —CO—O— or —O—CO—. The above-described compound may be “X—CO—O—Z” or “X—O—CO—Z”.

In the present specification, an acid dissociation constant (pKa) represents a pKa in an aqueous solution, and is specifically a value determined by computation from a value based on a Hammett's substituent constant and database of publicly known literature values, using the following software package 1. Any of the pKa values described in the present specification indicates values determined by computation using the software package.

Software Package 1: Advanced Chemistry Development (ACD/Labs) Software V 8.14 for Solaris (1994-2007 ACD/Labs).

In addition, the pKa can also be determined by a molecular orbital computation method. Examples of a specific method therefor include a method for performing calculation by computing H⁺ dissociation free energy in an aqueous solution based on a thermodynamic cycle. With regard to a computation method for H⁺ dissociation free energy, the H⁺ dissociation free energy can be computed by, for example, density functional theory (DFT), but various other methods have been reported in literature and the like, and are not limited thereto. There are a plurality of software applications capable of performing DFT, and examples thereof include Gaussian 16.

As described above, the pKa in the present specification refers to a value determined by computation from a value based on a Hammett's substituent constant and database of publicly known literature values, using the software package 1, but in a case where the pKa cannot be calculated by the method, a value obtained by Gaussian 16 based on density functional theory (DFT) shall be adopted.

The pKa in the present specification refers to a “pKa in an aqueous solution” as described above, but in a case where the pKa in an aqueous solution cannot be calculated, a “pKa in a dimethyl sulfoxide (DMSO) solution” shall be adopted.

In the present specification, the “solid content” means components that form an actinic ray-sensitive or radiation-sensitive film, and does not include a solvent. In addition, even in a case where a component is liquid, the component is included in the solid content as long as the component forms the actinic ray-sensitive or radiation-sensitive film.

A method for producing an actinic ray-sensitive or radiation-sensitive resin composition of the embodiment of the present invention (hereinafter, also referred to as a method for producing a composition of the embodiment of the present invention) includes:
a step (step (X)) of mixing an onium salt compound (1) represented by Formula (1A) or Formula (1B) with a salt compound (2) represented by Formula (2) in a non-aqueous solvent (S) to obtain an onium salt compound (3) represented by Formula (3A) or Formula (3B),
a step (step (Y)) of obtaining an onium salt compound (A) represented by Formula (4A) or Formula (4B) from the onium salt compound (3), and
a step (step (Z)) of mixing the onium salt compound (A) with the resin (B) whose solubility in an alkali developer changes due to action of acid.

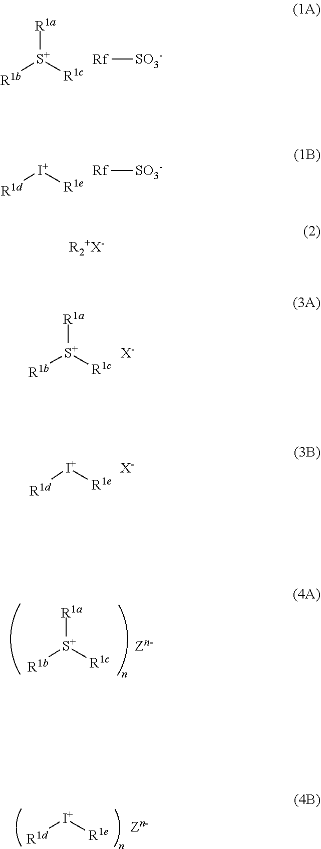

In Formulae (1A), (3A), and (4A), R^{1a}, R^{1b}, and R^{1c} each independently represent an alkyl group, a cycloalkyl group, an aryl group, or *—W₁—C(═O)Ar¹. Two of R^{1a}, R^{1b}, and R^{1c} may be bonded to each other to form a ring. *represents a linkage site to S⁺, W₁ represents a single bond or an alkylene group, and Ar¹ represents an aryl group.

In Formulae (1B), (3B), and (4B), R^{1d} and R^{1e} each independently represent an alkyl group, a cycloalkyl group, or an aryl group.

In Formulae (1A) and (1B), Rf represents an alkyl group or an aryl group which includes one or more fluorine atoms.

In Formula (2), R₂⁺ represents an organic cation having no polymer structure. X⁻ represents Cl⁻ or Br⁻.

In Formulae (3A) and (3B), X⁻ represents Cl⁻ or Br⁻.

In Formulae (4A) and (4B), Zⁿ⁻ represents an n-valent organic anion. n represents an integer of 1 or more.

In addition, the present invention also relates to a method for producing an onium salt compound (A) for an actinic ray-sensitive or radiation-sensitive resin composition, including the step (X) and the step (Y) (hereinafter, also referred to as a method for producing an onium salt compound according to the embodiment of the present invention).

The reason why the composition obtained by the method for producing an actinic ray-sensitive or radiation-sensitive resin composition according to the embodiment of the present invention (hereinafter, also referred to as the composition according to the embodiment of the present invention) has excellent LWR performance is not always clear, but the present inventors have presumed as follows.

According to the studies by the present inventors, it was found that in a case where there are many impurities such as raw material compounds and synthetic intermediates having fluorine-containing anions such as TfO⁻ contained in an onium salt compound in a composition, the LWR performance is deteriorated due to these impurities in the formation of a microfine pattern. Since these impurities can function as an acid or an acid diffusion control agent, it is presumed that these impurities affect acid diffusion control and lead to a decrease in LWR performance. In the onium salt compound obtained by the method for producing described in JP2014-97969A, since the residual rate of such impurities is high and the polarity of the onium salt compound is close to the polarity of the target onium salt compound, it is difficult to remove these impurities. In a case where an onium salt compound having a fluorine-containing anion such as TfO⁻ is used as a raw material, a method of obtaining a target onium salt compound through ion exchange using an ion exchange resin can also be used, but it is difficult to apply the method on an industrial scale from the viewpoint of work efficiency and the like.

In the method for producing an onium salt compound according to the embodiment of the present invention, by using a salt compound as a salt compound of an organic cation and a bromine ion or a chlorine ion for salt exchange with a raw material compound having a fluorine-containing anion such as TfO⁻, and further carrying out the salt exchange reaction of the raw material compound, which is usually carried out in a two-phase system, in a one-phase system using a non-aqueous solvent (S), the residual rate of impurities such as the raw material compound and the synthetic intermediate in the onium salt compound (A), which is a final product, can be reduced. Therefore, it is considered that the composition obtained by mixing the onium salt compound (A) obtained as described above with the resin (B) whose solubility in a developer changes due to an action of an acid, that is, the composition obtained by the method for producing a composition according to the embodiment of the present invention has few impurities in the onium salt compound and has excellent LWR performance even in the formation of an extremely fine pattern.

### [Actinic Ray-Sensitive or Radiation-Sensitive Resin Composition]

Hereinafter, various components used in the actinic ray-sensitive or radiation-sensitive resin composition obtained by the method for producing a composition according to the embodiment of the present invention (hereinafter, also referred to as the composition according to the embodiment of the present invention) will be described.

The composition of the embodiment of the present invention is typically a resist composition, and may be a positive tone resist composition or a negative tone resist composition. The composition of the embodiment of the present invention may be a resist composition for alkali development or a resist composition for organic solvent development.

The composition of the embodiment of the present invention may be a chemically amplified resist composition or a non-chemically amplified resist composition. The composition according to the embodiment of the present invention is typically a chemically amplified resist composition.

An actinic ray-sensitive or radiation-sensitive film can be formed using the composition of the embodiment of the present invention. The actinic ray-sensitive or radiation-sensitive film formed of the composition of the embodiment of the present invention is typically a resist film.

### [Onium Salt Compound (A)]

First, an onium salt compound (A) produced by the method for producing an onium salt compound according to the embodiment of the present invention will be described, and then the method for producing the onium salt compound (A) will be described for each step.

The onium salt compound (A) is a compound represented by Formula (4A) or Formula (4B).

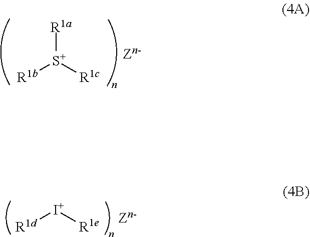

In Formula (4A), R^{1a}, R^{1b}, and R^{1c} each independently represent an alkyl group, a cycloalkyl group, an aryl group, or *—W₁—C(═O)Ar¹. Two of R^{1a}, R^{1b}, and R^{1c} may be bonded to each other to form a ring. *represents a linkage site to S⁺, W₁ represents a single bond or an alkylene group, and Ar¹ represents an aryl group.

In Formula (4B), R^{1d} and R^{1e} each independently represent an alkyl group, a cycloalkyl group, or an aryl group.

In Formulae (4A) and (4B), Zⁿ⁻ represents an n-valent organic anion. n represents an integer of 1 or more.

In Formula (4A), R^{1a}, R^{1b}, and R^{1c} each independently represent an alkyl group, a cycloalkyl group, an aryl group, or *—W₁—C(═O)Ar¹.

The number of carbon atoms in the alkyl group, the cycloalkyl group, and the aryl group as R^{1a}, R^{1b}, and R^{1c}, and the aryl group as Ar¹ in *—W₁—C(═O)Ar¹ is preferably 1 to 30 and more preferably 1 to 20. The alkyl group as R^{1a}, R^{1b}, and R^{1c} may be linear or branched.

The number of carbon atoms in the cycloalkyl group as R^{1a}, R^{1b}, and R^{1c} is preferably 3 to 30 and more preferably 3 to 20. The cycloalkyl group as R^{1a}, R^{1b}, and R^{1c} may be monocyclic or polycyclic.

In addition, the number of carbon atoms in the aryl group as R^{1a}, R^{1b}, and R^{1c} and the aryl group as Ar¹ in *—W₁—C(═O)Ar¹ is preferably 6 to 30 and more preferably 6 to 20. The aryl group as R^{1a}, R^{1b}, and R^{1c} and the aryl group as Ar¹ in *—W₁—C(═O)Ar¹ may be monocyclic or polycyclic.

W₁ represents a single bond or an alkylene group. The alkylene group of W₁ may be linear or branched, and is preferably an alkylene group having 1 to 10 carbon atoms and more preferably an alkylene group having 1 to 6 carbon atoms.

The alkyl group, the cycloalkyl group, and the aryl group as R^{1a}, R^{1b}, and R^{1c}, and the aryl group as Ar¹ in *—W₁—C(═O)Ar¹ may further have a substituent.

Two of R^{1a}, R^{1b}, and R^{1c} may be bonded to each other to form a ring structure, and the ring may include an oxygen atom, a sulfur atom, an ester group, an amide group, or a carbonyl group. Examples of the group formed by the bonding of two of R^{1a}, R^{1b}, and R^{1c} include an alkylene group (for example, a butylene group and a pentylene group) and —CH₂—CH₂—O—CH₂—CH₂—.

In a case where the electron withdrawing property of R^{1a}, R^{1b}, and R^{1c} is too high, the resistance of the cation to a nucleophile or heating may be lowered, which may cause undesirable decomposition in a synthesis process or a pattern forming step. Therefore, in a case where R^{1a}, R^{1b}, and R^{1c} represent an alkyl group, it is preferable that the alkyl group does not include a halogen atom. That is, in Formula (4A), R^{1a}, R^{1b}, and R^{1c} preferably each independently represent an alkyl group which does not include a halogen atom, a cycloalkyl group, an aryl group, or *—W₁—C(═O)Ar¹.

Suitable aspects of the sulfonium cation contained in the onium salt compound represented by Formula (4A) include a cation (ZaI-1), a cation (ZaI-2), a cation (ZaI-3b), and a cation (ZaI-4b), which will be described later.

First, the cation (ZaI-1) will be described.

The cation (ZaI-1) is an arylsulfonium cation in which at least one of R^{1a}, R^{1b}, or R^{1c} in Formula (4A) is an aryl group.

In the arylsulfonium cation, all of R^{1a}, R^{1b}, and R^{1c} may be aryl groups, or some of R^{1a}, R^{1b}, and R^{1c} may be aryl groups, and the rest may be an alkyl group, a cycloalkyl group, or *—W₁—C(═O)Ar¹.

One of R^{1a}, R^{1b}, or R^{1c} is an aryl group, and the remaining two of R^{1a}, R^{1b}, and R^{1c} may be bonded to each other to form a ring structure, where the ring may include an oxygen atom, a sulfur atom, an ester group, an amide group, or a carbonyl group. Examples of the group formed by the bonding of two of R^{1a}, R^{1b}, and R^{1c} include an alkylene group (for example, a butylene group, a pentylene group, and —CH₂—CH₂—O—CH₂—CH₂—) in which one or more methylene groups may be substituted with an oxygen atom, a sulfur atom, an ester group, an amide group, and/or a carbonyl group.

Examples of the arylsulfonium cation include a triarylsulfonium cation, a diarylalkylsulfonium cation, an aryldialkylsulfonium cation, a diarylcycloalkylsulfonium cation, and an aryldicycloalkylsulfonium cation.

It is preferable that all of R^{1a}, R^{1b}, and R^{1c} are aryl groups. Two of R^{1a}, R^{1b}, and R^{1c} may be bonded to each other to form a ring. That is, the arylsulfonium cation is preferably a triarylsulfonium cation.

The aryl group included in the arylsulfonium cation is preferably a phenyl group or a naphthyl group, and more preferably a phenyl group. The aryl group may be an aryl group which has a heterocyclic structure having an oxygen atom, a nitrogen atom, a sulfur atom, or the like. Examples of the heterocyclic structure include a pyrrole residue, a furan residue, a thiophene residue, an indole residue, a benzofuran residue, and a benzothiophene residue. In a case where the arylsulfonium cation has two or more aryl groups, the two or more aryl groups may be the same or different from each other.

The alkyl group or the cycloalkyl group included in the arylsulfonium cation as necessary is preferably a linear alkyl group having 1 to 15 carbon atoms, a branched alkyl group having 3 to 15 carbon atoms, or a cycloalkyl group having 3 to 15 carbon atoms, and more preferably a methyl group, an ethyl group, a propyl group, an n-butyl group, a sec-butyl group, a t-butyl group, a cyclopropyl group, a cyclobutyl group, or a cyclohexyl group.

Ar¹ in *—W₁—C(═O)Ar¹ which is contained in the arylsulfonium cation as necessary is preferably the group mentioned as the aryl group contained in the above-described arylsulfonium cation.

As the substituent which may be contained in the aryl group, the alkyl group, the cycloalkyl group, and *—W₁—C(═O)Ar¹ of R^{1a}, R^{1b}, and R^{1c}, an alkyl group (for example, having 1 to 15 carbon atoms), a cycloalkyl group (for example, having 3 to 15 carbon atoms), an aryl group (for example, having 6 to 14 carbon atoms), an alkoxy group (for example, having 1 to 15 carbon atoms), a cycloalkylalkoxy group (for example, having 1 to 15 carbon atoms), a halogen atom (for example, fluorine and iodine), a hydroxyl group, a carboxyl group, an ester group, a sulfinyl group, a sulfonyl group, an alkylthio group, or a phenylthio group is preferable.

The substituent may further have a substituent if possible, and it is also preferable that the above-described alkyl group has a halogen atom as the substituent to form an alkyl halide group such as a trifluoromethyl group.

However, as described above, in a case where R^{1a}, R^{1b}, or R^{1c} represents an alkyl group, it is preferable that the substituent does not contain a halogen atom.

It is also preferable that the substituents form an acid-decomposable group by any combination.

The acid-decomposable group is intended to a group which is decomposed by action of acid to generate a polar group, and preferably has a structure in which a polar group is protected by a group which is eliminated by action of acid. Examples of the above-described polar group and leaving group include a polar group and a leaving group described later in a repeating unit having an acid-decomposable group of the resin (B) described later.

Next, the cation (ZaI-2) will be described.

The cation (ZaI-2) is a cation in which R^{1a}, R^{1b}, and R^{1c} in Formula (4A) each independently represent an alkyl group or a cycloalkyl group and represent a cation having no aromatic ring. The aromatic ring also encompasses an aromatic ring including a heteroatom.

The number of carbon atoms in the alkyl group and the cycloalkyl group as R^{1a}, R^{1b}, and R^{1c} is preferably 1 to 30 and more preferably 1 to 20.

Examples of the alkyl group and cycloalkyl group of R^{1a}, R^{1b}, and R^{1c} include a linear alkyl group having 1 to 10 carbon atoms or a branched alkyl group having 3 to 10 carbon atoms (for example, a methyl group, an ethyl group, a propyl group, a butyl group, and a pentyl group), and a cycloalkyl group having 3 to 10 carbon atoms (for example, a cyclopentyl group, a cyclohexyl group, and a norbornyl group).

R^{1a}, R^{1b}, and R^{1c} may be further substituted with a halogen atom, an alkoxy group (for example, an alkoxy group having 1 to 5 carbon atoms), a hydroxyl group, a cyano group, or a nitro group.

However, as described above, in a case where R^{1a}, R^{1b}, or R^{1c} represents an alkyl group, it is preferable that the substituent does not contain a halogen atom.

It is also preferable that the substituents of R^{1a}, R^{1b}, and R^{1c} each independently form an acid-decomposable group by any combination of the substituents.

Next, the cation (ZaI-3b) will be described.

The cation (ZaI-3b) is a cation represented by Formula (ZaI-3b).

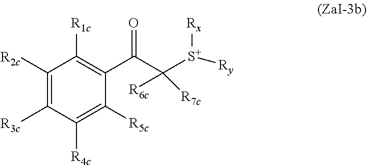

In Formula (ZaI-3b), R_{1c} to R_{5c} each independently represent a hydrogen atom, an alkyl group, a cycloalkyl group, an aryl group, an alkoxy group, an aryloxy group, an alkoxycarbonyl group, an alkylcarbonyloxy group, a cycloalkylcarbonyloxy group, a halogen atom, a hydroxyl group, a nitro group, an alkylthio group, or an arylthio group.

R_{6c} and R_{7c} each independently represent a hydrogen atom, an alkyl group (for example, a t-butyl group and the like), a cycloalkyl group, a halogen atom, a cyano group, or an aryl group.

Rₓ and R_{y} each independently represent an alkyl group or a cycloalkyl group.

It is also preferable that the substituents of R_{1c} to R_{7c}, Rₓ, and R_{y} each independently form an acid-decomposable group by any combination of the substituents.

Any two or more of R_{1c}, . . . , or R_{5c}, R_{5c} and R_{6c}, R_{6c} and R_{7c}, R_{5c} and Rₓ, and Rₓ and R_{y} may each be bonded to each other to form a ring, and the rings may each independently include an oxygen atom, a sulfur atom, a ketone group, an ester bond, or an amide bond.

Examples of the above-described ring include an aromatic or non-aromatic hydrocarbon ring, an aromatic or non-aromatic heterocyclic ring, and a polycyclic fused ring formed by a combination of two or more of these rings. Examples of the ring include a 3- to 10-membered ring, and the ring is preferably a 4- to 8-membered ring and more preferably a 5- or 6-membered ring.

Examples of the group formed by the bonding of any two or more of R_{1c}, . . . , or R_{5c}, R_{6c} and R_{7c}, and Rₓ and R_{y} include an alkylene group such as a butylene group and a pentylene group. A methylene group in this alkylene group may be substituted with a heteroatom such as an oxygen atom.

As the group formed by the bonding of R_{5c} and R_{6c}, and R_{5c} and Rₓ, a single bond or an alkylene group is preferable. Examples of the alkylene group include a methylene group and an ethylene group.

The ring formed by bonding R_{1c} to R_{5c}, R_{6c}, R_{7c}, Rₓ, R_{y}, any two or more of R_{1c}, . . . , or R_{5c}, R_{5c} and R_{6c}, R_{6c} and R_{7c}, R_{5c} and Rₓ, and Rₓ and R_{y} to each other may have a substituent.

Next, the cation (ZaI-4b) will be described.

The cation (ZaI-4b) is a cation represented by Formula (ZaI-4b).

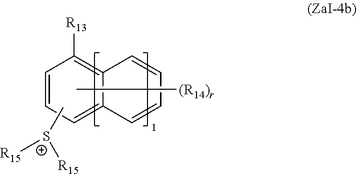

In Formula (ZaI-4b), 1 represents an integer of 0 to 2, and r represents an integer of 0 to 8.

R₁₃ represents a hydrogen atom, a halogen atom (for example, a fluorine atom, an iodine atom, or the like), a hydroxyl group, an alkyl group, an alkyl halide group, an alkoxy group, a carboxyl group, an alkoxycarbonyl group, or a group having a cycloalkyl group (which may be the cycloalkyl group itself or a group including the cycloalkyl group in a part thereof). These groups may have a substituent.

R₁₄ represents a hydroxyl group, a halogen atom (for example, a fluorine atom, an iodine atom, or the like), an alkyl group, an alkyl halide group, an alkoxy group, an alkoxycarbonyl group, an alkylcarbonyl group, an alkylsulfonyl group, a cycloalkylsulfonyl group, or a group including a cycloalkyl group (which may be the cycloalkyl group itself or a group including the cycloalkyl group in a part thereof). These groups may have a substituent. In a case of a plurality of R₁₄'s, R₁₄'s each independently represent the above-described group such as a hydroxyl group.

R₁₅'s each independently represent an alkyl group, a cycloalkyl group, or a naphthyl group. Two R₁₅'s may be bonded to each other to form a ring. In a case where two R₁₅'s are bonded to each other to form a ring, the ring skeleton may include a heteroatom such as an oxygen atom and a nitrogen atom.

In one aspect, it is preferable that two R₁₅'s are alkylene groups and are bonded to each other to form a ring structure. The above-described alkyl group, the above-described cycloalkyl group, the above-descried naphthyl group, and the ring formed by bonding two R₁₅'s to each other may have a substituent.

In Formula (ZaI-4b), the alkyl group of R₁₃, R₁₄, and R₁₅ may be linear or branched. The number of carbon atoms in the alkyl group is preferably 1 to 10. The alkyl group is preferably a methyl group, an ethyl group, an n-butyl group, a t-butyl group, or the like.

It is also preferable that the substituents of R₁₃ to R₁₅, Rₓ, and R_{y} each independently form an acid-decomposable group by any combination of the substituents.

Next, R^{1d} and R^{1e} in Formula (4B) will be described.

In Formula (4B), R^{1d} and R^{1e} each independently represent an alkyl group, a cycloalkyl group, or an aryl group.

The aryl group of R^{1d} and R^{1e} is preferably a phenyl group or a naphthyl group, and more preferably a phenyl group. The aryl group of R^{1d} and R^{1e} may be an aryl group which has a heterocyclic ring having an oxygen atom, a nitrogen atom, a sulfur atom, or the like. Examples of a skeleton of the aryl group having a heterocyclic ring include pyrrole, furan, thiophene, indole, benzofuran, and benzothiophene.

The alkyl group and cycloalkyl group of R^{1d} and R^{1e} are preferably a linear alkyl group having 1 to 10 carbon atoms or a branched alkyl group having 3 to 10 carbon atoms (for example, a methyl group, an ethyl group, a propyl group, a butyl group, and a pentyl group), or a cycloalkyl group having 3 to 10 carbon atoms (for example, a cyclopentyl group, a cyclohexyl group, and a norbornyl group).

The alkyl group, the cycloalkyl group, and the aryl group of R^{1d} and R^{1e} may each independently have a substituent. Examples of the substituent which may be included in each of the aryl group, the alkyl group, and the cycloalkyl group of R^{1d} and R^{1e} include an alkyl group (for example, having 1 to 15 carbon atoms), a cycloalkyl group (for example, having 3 to 15 carbon atoms), an aryl group (for example, having 6 to 15 carbon atoms), an alkoxy group (for example, having 1 to 15 carbon atoms), a halogen atom, a hydroxyl group, and a phenylthio group. In addition, it is also preferable that the substituents of R^{1d} and R^{1e} each independently form an acid-decomposable group by any combination of the substituents.

Specific examples of the sulfonium cation contained in the onium salt compound represented by Formula (4A) and the iodonium cation contained in the onium salt compound represented by Formula (4B) will be shown below, but the present invention is not limited thereto.

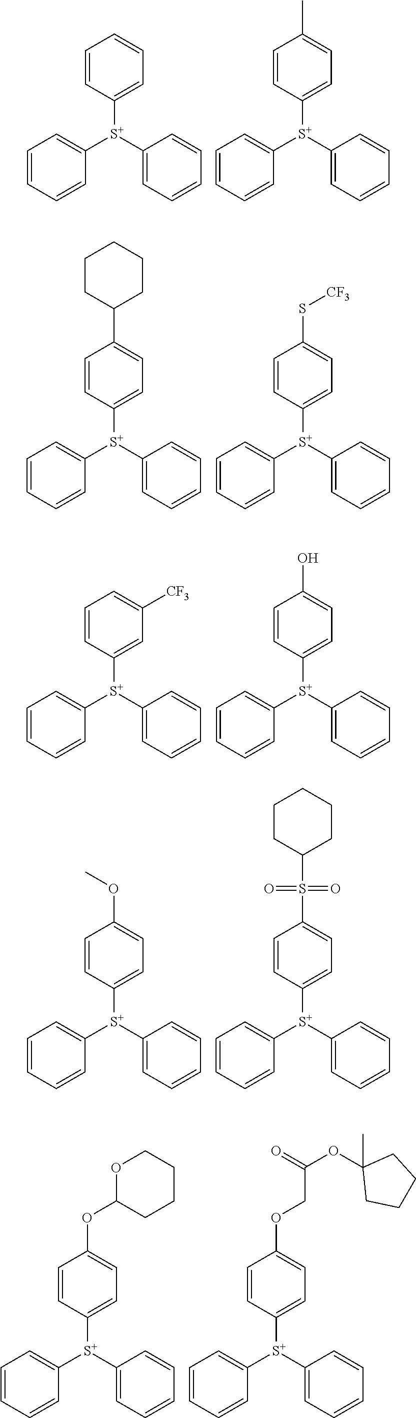

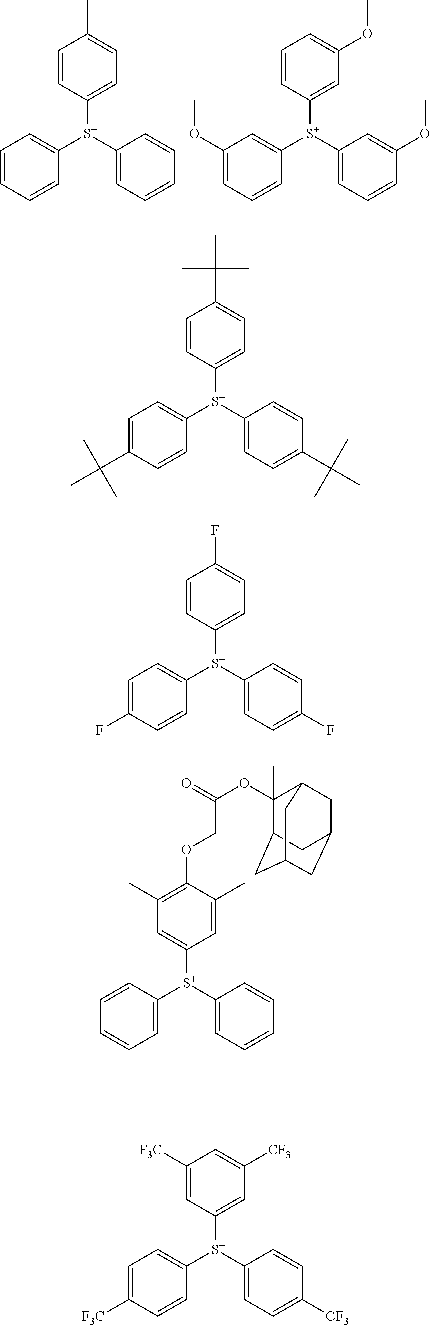 [SMILES: Cc1ccc([S+](c2ccccc2)c2ccccc2)cc1 ; COc1cccc([S+](c2cccc(OC)c2)c2cccc(OC)c2)c1 ; CC(C)(C)c1ccc([S+](c2ccc(C(C)(C)C)cc2)c2ccc(C(C)(C)C)cc2)cc1 ; Fc1ccc([S+](c2ccc(F)cc2)c2ccc(F)cc2)cc1 ; Cc1cc([S+](c2ccccc2)c2ccccc2)cc(C)c1OCC(=O)OC1(C)C2CC3CC(C2)CC1C3 ; FC(F)(F)c1ccc([S+](c2ccc(C(F)(F)F)cc2)c2cc(C(F)(F)F)cc(C(F)(F)F)c2)cc1]

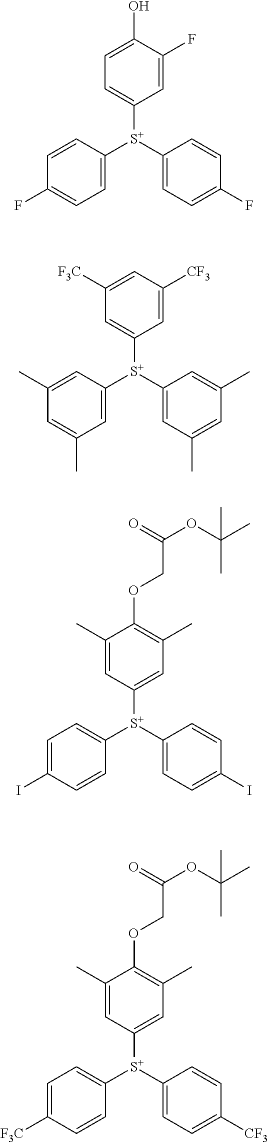

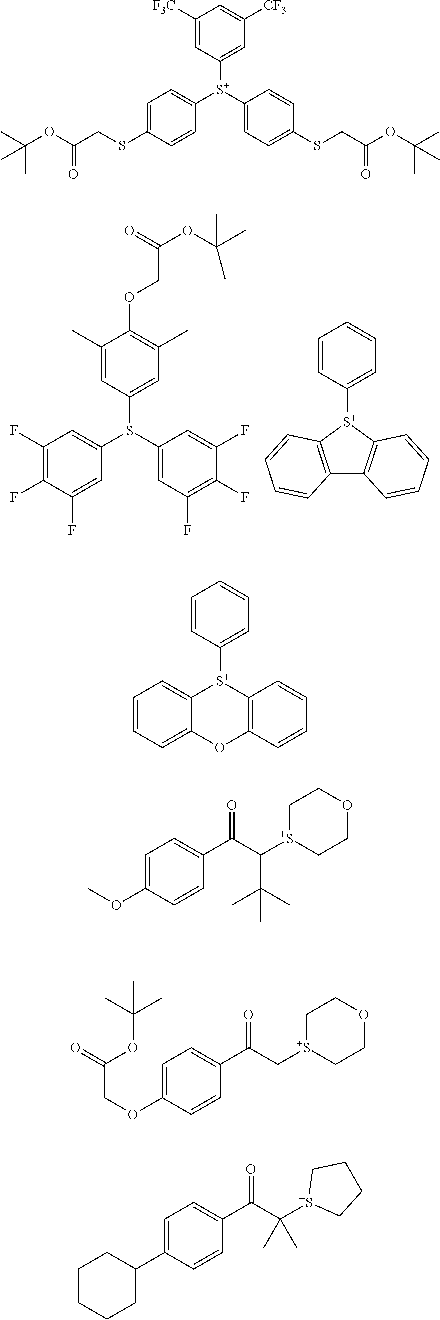

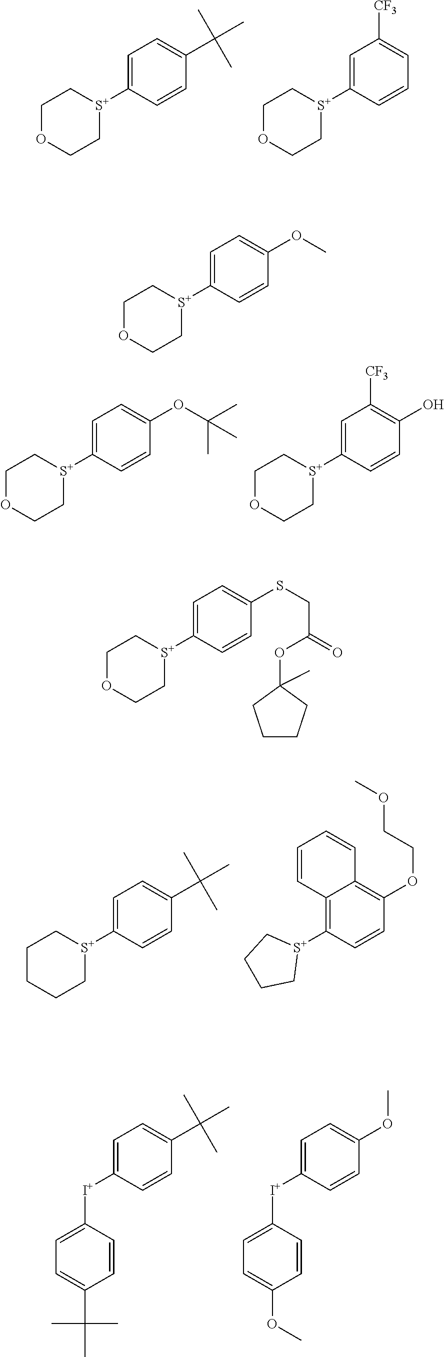

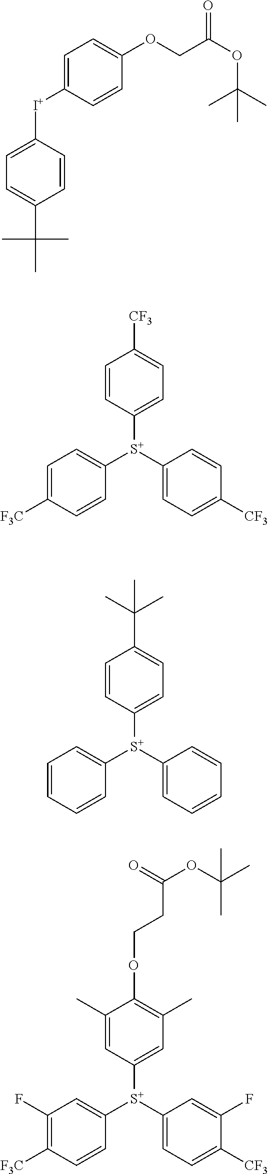 [SMILES: CC(C)(C)OC(=O)COc1ccc([I+]c2ccc(C(C)(C)C)cc2)cc1 ; FC(F)(F)c1ccc([S+](c2ccc(C(F)(F)F)cc2)c2ccc(C(F)(F)F)cc2)cc1 ; CC(C)(C)c1ccc([S+](c2ccccc2)c2ccccc2)cc1 ; Cc1cc([S+](c2ccc(C(F)(F)F)c(F)c2)c2ccc(C(F)(F)F)c(F)c2)cc(C)c1OCCC(=O)OC(C)(C)C]

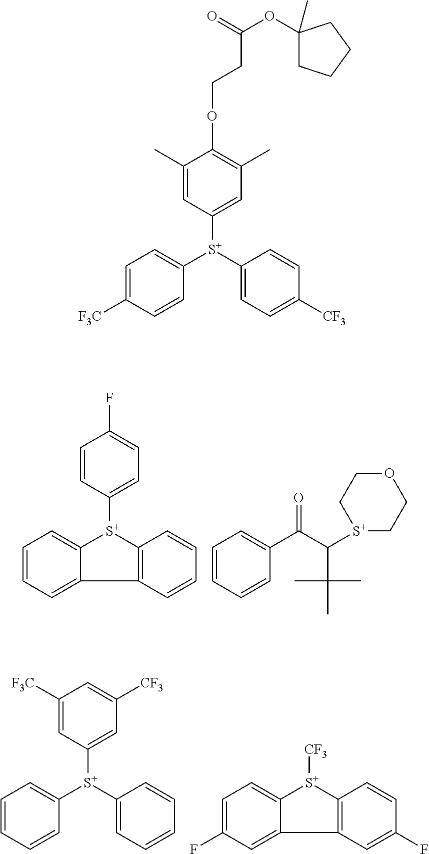 [SMILES: Cc1cc([S+](c2ccc(C(F)(F)F)cc2)c2ccc(C(F)(F)F)cc2)cc(C)c1OCCC(=O)OC1(C)CCCC1 ; Fc1ccc(-[s+]2c3ccccc3c3ccccc32)cc1 ; CC(C)(C)C(C(=O)c1ccccc1)[S+]1CCOCC1 ; FC(F)(F)c1cc([S+](c2ccccc2)c2ccccc2)cc(C(F)(F)F)c1 ; Fc1ccc2c(c1)c1cc(F)ccc1[s+]2C(F)(F)F]

The number of sulfonium cations in the onium salt compound represented by Formula (4A) and the number of iodonium cations in the onium salt compound represented by Formula (4B) may be 1 or 2 or more. That is, n may be 1 or 2 or more. In a case where n represents an integer of 2 or more, a plurality of the cations may be the same or different from each other, but are preferably the same.

n in Formulae (4A) and (4B) is preferably 1 to 4 and more preferably 1 to 3.

Zⁿ⁻ in Formulae (4A) and (4B) represents an n-valent organic anion.

The organic anion is not particularly limited, and examples thereof include a monovalent or di- or higher valent organic anion.

The organic anion is preferably an anion with significantly lower ability to undergo nucleophilic reaction, and more preferably a non-nucleophilic anion.

Examples of the non-nucleophilic anion include a sulfonate anion (an aliphatic sulfonate anion, an aromatic sulfonate anion, a camphor sulfonate anion, and the like), a carboxylate anion (an aliphatic carboxylate anion, an aromatic carboxylate anion, an aralkyl carboxylate anion, and the like), a sulfonylimide anion, a bis(alkylsulfonyl)imide anion, and a tris(alkylsulfonyl)methide anion.

An aliphatic moiety in the aliphatic sulfonate anion and the aliphatic carboxylate anion may be a linear or branched alkyl group or a cycloalkyl group, and a linear or branched alkyl group having 1 to 30 carbon atoms or a cycloalkyl group having 3 to 30 carbon atoms is preferable.

The alkyl group may be, for example, a fluoroalkyl group (which may have a substituent other than a fluorine atom, and may be a perfluoroalkyl group).

An aryl group in the aromatic sulfonate anion and the aromatic carboxylate anion is preferably an aryl group having 6 to 14 carbon atoms, and examples thereof include a phenyl group, a tolyl group, and a naphthyl group.

The alkyl group, cycloalkyl group, and aryl group mentioned above may have a substituent. The substituent is not particularly limited, and examples thereof include a nitro group, a halogen atom such as a fluorine atom and a chlorine atom, a carboxyl group, a hydroxyl group, an amino group, a cyano group, an alkoxy group (preferably having 1 to 15 carbon atoms), an alkyl group (preferably having 1 to 10 carbon atoms), a cycloalkyl group (preferably having 3 to 15 carbon atoms), an aryl group (preferably having 6 to 14 carbon atoms), an alkoxycarbonyl group (preferably having 2 to 7 carbon atoms), an acyl group (preferably having 2 to 12 carbon atoms), an alkoxycarbonyloxy group (preferably having 2 to 7 carbon atoms), an alkylthio group (preferably having 1 to 15 carbon atoms), an alkylsulfonyl group (preferably having 1 to 15 carbon atoms), an alkyliminosulfonyl group (preferably having 1 to 15 carbon atoms), and an aryloxysulfonyl group (preferably having 6 to 20 carbon atoms).

As the aralkyl group in the aralkyl carboxylate anion, an aralkyl group having 7 to 14 carbon atoms is preferable.

Examples of the aralkyl group having 7 to 14 carbon atoms include a benzyl group, a phenethyl group, a naphthylmethyl group, a naphthylethyl group, and a naphthylbutyl group.

Examples of the sulfonylimide anion include a saccharin anion.

As the alkyl group in the bis(alkylsulfonyl)imide anion and the tris(alkylsulfonyl)methide anion, an alkyl group having 1 to 5 carbon atoms is preferable. Examples of a substituent of these alkyl group include a halogen atom, an alkyl group substituted with a halogen atom, an alkoxy group, an alkylthio group, an alkyloxysulfonyl group, an aryloxysulfonyl group, and a cycloalkylaryloxysulfonyl group, and a fluorine atom or an alkyl group substituted with a fluorine atom is preferable.

In addition, the alkyl groups in the bis(alkylsulfonyl)imide anion may be bonded to each other to form a ring structure. As a result, acid strength is increased.

Examples of other non-nucleophilic anions include fluorinated phosphorus (for example, PF₆⁻), fluorinated boron (for example, BF₄⁻), and fluorinated antimony (for example, SbF₆⁻).

As the non-nucleophilic anion, an aliphatic sulfonate anion in which at least an α-position of the sulfonic acid is substituted with a fluorine atom, an aromatic sulfonate anion substituted with a fluorine atom or a group having a fluorine atom, a bis(alkylsulfonyl)imide anion in which an alkyl group is substituted with a fluorine atom, or a tris(alkylsulfonyl)methide anion in which an alkyl group is substituted with a fluorine atom is preferable. Among these, a perfluoroaliphatic sulfonate anion (preferably having 4 to 8 carbon atoms) or benzenesulfonate anion having a fluorine atom is more preferable, and a nonafluorobutanesulfonate anion, a perfluorooctanesulfonate anion, a pentafluorobenzenesulfonate anion, or a 3,5-bis(trifluoromethyl)benzenesulfonate anion is still more preferable.

As the non-nucleophilic anion, an anion represented by Formula (AN1) is also preferable.

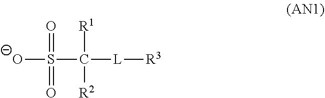

In Formula (AN1), R¹ and R² each independently represent a hydrogen atom or a substituent.

The substituent is not particularly limited, but a group which is not an electron withdrawing group is preferable. Examples of the group which is not an electron withdrawing group include a hydrocarbon group, a hydroxyl group, an oxyhydrocarbon group, an oxycarbonyl hydrocarbon group, an amino group, a hydrocarbon-substituted amino group, and a hydrocarbon-substituted amide group.

In addition, the groups which are not an electron withdrawing group are each independently preferably —R′, —OH, —OR′, —OCOR′, —NH₂, —NR′₂, —NHR′, or —NHCOR′. R′ is a monovalent hydrocarbon group.

Examples of the above-described monovalent hydrocarbon group represented by R′ include an alkyl group such as a methyl group, an ethyl group, a propyl group, and a butyl group; an alkenyl group such as an ethenyl group, a propenyl group, and a butenyl group; a monovalent linear or branched hydrocarbon group of an alkynyl group or the like, such as an ethynyl group, a propynyl group, and a butynyl group; a cycloalkyl group such as a cyclopropyl group, a cyclobutyl group, a cyclopentyl group, a cyclohexyl group, a norbornyl group, and an adamantyl group; a monovalent alicyclic hydrocarbon group of a cycloalkenyl group or the like, such as a cyclopropenyl group, a cyclobutenyl group, a cyclopentenyl group, and a norbornenyl group; an aryl group such as a phenyl group, a tolyl group, a xylyl group, a mesityl group, a naphthyl group, a methylnaphthyl group, an anthryl group, and a methylanthryl group; and a monovalent aromatic hydrocarbon group of an aralkyl group or the like, such as a benzyl group, a phenethyl group, a phenylpropyl group, a naphthylmethyl group, and an anthrylmethyl group.

Among these, R¹ and R² are each independently a hydrocarbon group (preferably, a cycloalkyl group) or a hydrogen atom.

L represents a divalent linking group.

In a case of a plurality of L's, L's may be the same or different from each other.

Examples of the divalent linking group include —O—CO—O—, —COO—, —CONH—, —CO—, —O—, —S—, —SO—, —SO₂—, an alkylene group (preferably having 1 to 6 carbon atoms), a cycloalkylene group (preferably having 3 to 15 carbon atoms), an alkenylene group (preferably having 2 to 6 carbon atoms), and a divalent linking group formed by a combination of a plurality of these groups. Among these, as the divalent linking group, —O—CO—O—, —COO—, —CONH—, —CO—, —O—, —SO₂—, —O—CO—O-alkylene group-, —COO-alkylene group-, or —CONH-alkylene group-, and more preferably —O—CO—O—, —O—CO—O-alkylene group-, —COO—, —CONH—, —SO₂—, or —COO-alkylene group-.

For example, L is preferably a group represented by Formula (AN1-1).

*^{a}—(CR^{2a}₂)_{X}-Q-(CR^{2b}₂)_{Y}—*^{b} (AN1-1)

In Formula (AN1-1), *^{a} represents a bonding position with R³ in Formula (AN1).

*^{b} represents a bonding position —C(R¹)(R²)— in Formula (AN1).

X and Y each independently represent an integer of 0 to 10, preferably an integer of 0 to 3.

R^{2a} and R^{2b} each independently represent a hydrogen atom or a substituent.

In a case where R^{2a}'s and R^{2b}'s are present in a plural number, the R^{2a}'s and R^{2b}'s which are present in a plural number may be the same or different from each other.

However, in a case where Y is 1 or more, R^{2b} in CR^{2b}₂ which is directly bonded to —C(R¹)(R²)— in Formula (AN1) is not a fluorine atom.

Q represents *^{A}—O—CO—O—*^{B}, *^{A}—CO—*^{B}, *^{A}—CO—O—*^{B}, *^{A}—O—CO—*^{B}, *^{A}—O—*^{B}, *^{A}—S—*^{B} or *^{A}—SO₂—*^{B} However, in a case where X+Y in Formula (AN1-1) is 1 or more and R^{2a} and R^{2b} in Formula (AN1-1) are all hydrogen atoms, Q represents *^{A}—O—CO—O—*^{B}, *^{A}—CO—*^{B}, *^{A}—O—CO—*^{B}, *^{A}—O—*^{B}, *^{A}—S—*^{B}, or *^{A}—SO₂—*^{B}.

*^{A} represents a bonding position on the R³ side in Formula (AN1), and *^{B} represents a bonding position on the —SO3⁻ side in Formula (AN1).

In Formula (AN1), R³ represents an organic group.

The above-described organic group is not particularly limited as long as it has one or more carbon atoms, and may be a linear group (for example, a linear alkyl group) or a branched group (for example, a branched alkyl group such as a t-butyl group), and may be a cyclic group. The above-described organic group may or may not have a substituent. The above-described organic group may or may not have a heteroatom (oxygen atom, sulfur atom, nitrogen atom, and/or the like).

Among these, R³ is preferably an organic group having a cyclic structure. The above-described cyclic structure may be monocyclic or polycyclic, and may have a substituent. The ring of the organic group including a cyclic structure is preferably directly bonded to L in Formula (AN1).

For example, the above-described organic group having a cyclic structure may or may not have a heteroatom (oxygen atom, sulfur atom, nitrogen atom, and/or the like). The heteroatom may be substituted on one or more carbon atoms forming the cyclic structure.

As the above-described organic group having a cyclic structure, for example, a hydrocarbon group having a cyclic structure, a lactone ring group, or a sultone ring group is preferable. Among these, the above-described organic group having a cyclic structure is preferably a hydrocarbon group having a cyclic structure.

The above-described hydrocarbon group having a cyclic structure is preferably a monocyclic or polycyclic cycloalkyl group. These groups may have a substituent.

The above-described cycloalkyl group may be a monocycle (cyclohexyl group or the like) or a polycycle (adamantyl group or the like), and the number of carbon atoms is preferably 5 to 12.

As the above-described lactone group and sultone group, for example, a group obtained by removing one hydrogen atom from ring member atoms constituting the lactone structure or the sultone structure in any of the structures represented by Formulae (LC1-1) to (LC1-21) described above and the structures represented by Formulae (SL1-1) to (SL1-3) described above is preferable.

The non-nucleophilic anion may be a benzenesulfonate anion, and is preferably a benzenesulfonate anion substituted with a branched alkyl group or a cycloalkyl group.

As the non-nucleophilic anion, an anion represented by Formula (AN2) is also preferable.

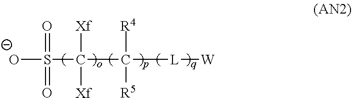

In Formula (AN2), o represents an integer of 1 to 3. p represents an integer of 0 to 10. q represents an integer of 0 to 10.

Xf represents a hydrogen atom, a fluorine atom, an alkyl group substituted with at least one fluorine atom, or an organic group not having a fluorine atom. The number of carbon atoms in the alkyl group is preferably 1 to 10 and more preferably 1 to 4. The alkyl group substituted with at least one fluorine atom is preferably a perfluoroalkyl group.

Xf is preferably a fluorine atom or a perfluoroalkyl group having 1 to 4 carbon atoms and more preferably a fluorine atom or CF₃, and it is still more preferable that both Xf's are fluorine atoms.

R⁴ and R⁵ each independently represent a hydrogen atom, a fluorine atom, an alkyl group, or an alkyl group substituted with at least one fluorine atom. In a case of a plurality of R⁴'s and R⁵'s, R⁴'s and R⁵'s may be the same or different from each other. The alkyl group represented by R⁴ and R⁵ preferably has 1 to 4 carbon atoms. The above-described alkyl group may further have a substituent. R₄ and R₅ are preferably a hydrogen atom.

L represents a divalent linking group. The definition of L is synonymous with L in Formula (AN1).

W represents an organic group including a cyclic structure. Among these, a cyclic organic group is preferable.

Examples of the cyclic organic group include an alicyclic group, an aryl group, and a heterocyclic group.

The alicyclic group may be monocyclic or polycyclic. Examples of the monocyclic alicyclic group include a monocyclic cycloalkyl group such as a cyclopentyl group, a cyclohexyl group, and a cyclooctyl group. Examples of the polycyclic alicyclic group include a polycyclic cycloalkyl group such as a norbornyl group, a tricyclodecanyl group, a tetracyclodecanyl group, a tetracyclododecanyl group, and an adamantyl group. Among these, an alicyclic group having a bulky structure with 7 or more carbon atoms, such as a norbornyl group, a tricyclodecanyl group, a tetracyclodecanyl group, a tetracyclododecanyl group, and an adamantyl group, is preferable.

The aryl group may be monocyclic or polycyclic. Examples of the above-described aryl group include a phenyl group, a naphthyl group, a phenanthryl group, and an anthryl group.

The heterocyclic group may be monocyclic or polycyclic. Among these, in a case of a polycyclic heterocyclic group, the diffusion of the acid can be further suppressed. The heterocyclic group may or may not have aromaticity. Examples of a heterocycle having aromaticity include a furan ring, a thiophene ring, a benzofuran ring, a benzothiophene ring, a dibenzofuran ring, a dibenzothiophene ring, and a pyridine ring. Examples of a heterocycle not having aromaticity include a tetrahydropyran ring, a lactone ring, a sultone ring, and a decahydroisoquinoline ring. As the heterocycle in the heterocyclic group, a furan ring, a thiophene ring, a pyridine ring, or a decahydroisoquinoline ring is preferable.

The above-described cyclic organic group may have a substituent. Examples of the above-described substituent include an alkyl group (may be linear or branched; preferably having 1 to 12 carbon atoms), a cycloalkyl group (may be monocyclic, polycyclic, or spirocyclic; preferably having 3 to 20 carbon atoms), an aryl group (preferably having 6 to 14 carbon atoms), a hydroxyl group, an alkoxy group, an ester group, an amide group, a urethane group, a ureido group, a thioether group, a sulfonamide group, and a sulfonic acid ester group. A carbon constituting the cyclic organic group (carbon contributing to ring formation) may be a carbonyl carbon.

As the anion represented by Formula (AN2), SO₃⁻—CF₂—CH₂—OCO-(L)_{q′}-W, SO₃⁻—CF₂—CHF—CH₂—OCO-(L)_{q′}—W, SO₃⁻—CF₂—COO-(L)_{q′}-W, SO₃⁻—CF₂—CF₂—CH₂—CH₂-(L)_{q}- W, or SO₃−—CF₂—CH(CF₃)OCO-(L)_{q′}-W is preferable. Here, L, q, and W are the same as in Formula (AN2). q′ represents an integer of 0 to 10.

As the non-nucleophilic anion, an aromatic sulfonate anion represented by Formula (AN3) is also preferable.

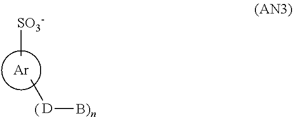

In Formula (AN3), Ar represents an aryl group (phenyl group or the like), and may further have a substituent other than a sulfonate anion and a -(D-B) group. Examples of the substituent which may be further included include a fluorine atom and a hydroxyl group.

n represents an integer of 0 or more. n is preferably 1 to 4, more preferably 2 or 3, and still more preferably 3.

D represents a single bond or a divalent linking group. Examples of the divalent linking group include an ether group, a thioether group, a carbonyl group, a sulfoxide group, a sulfone group, a sulfonic acid ester group, an ester group, and a group consisting of a combination of two or more of these groups.

B represents a hydrocarbon group.

B is preferably an aliphatic hydrocarbon group, and more preferably an isopropyl group, a cyclohexyl group, or an aryl group which may further have a substituent (such as a tricyclohexylphenyl group).

As the non-nucleophilic anion, a disulfonamide anion is also preferable.

The disulfonamide anion is, for example, an anion represented by N⁻(SO₂—R^{q})₂.

Here, R^{q} represents an alkyl group which may have a substituent, and is preferably a fluoroalkyl group and more preferably a perfluoroalkyl group. Two R^{q}'s may be bonded to each other to form a ring. The group formed by bonding two R^{q}'s to each other is preferably an alkylene group which may have a substituent, preferably a fluoroalkylene group, and still more preferably a perfluoroalkylene group. The number of carbon atoms in the above-described alkylene group is preferably 2 to 4.

In addition, examples of the non-nucleophilic anion include anions represented by Formulae (d1-1) to (d1-4).

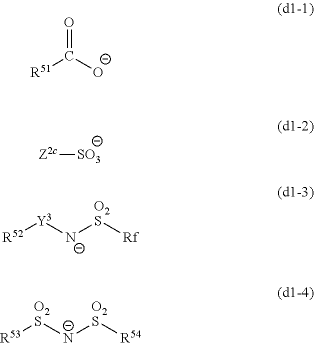

In Formula (d1-1), R⁵¹ represents a hydrocarbon group (for example, an aryl group such as a phenyl group) which may have a substituent (for example, a hydroxyl group).

In Formula (d1-2), Z^{2c} represents a hydrocarbon group having 1 to 30 carbon atoms, which may have a substituent (provided that a carbon atom adjacent to S is not substituted with a fluorine atom).

The above-described hydrocarbon group in Z^{2c} may be linear or branched, may have a cyclic structure. In addition, carbon atoms in the above-described hydrocarbon group (preferably, carbon atoms which are ring member atoms in a case where the above-described hydrocarbon group has a cyclic structure) may be a carbonyl carbon (—CO—). Examples of the hydrocarbon group include a group having a norbornyl group which may have a substituent. Carbon atoms forming the above-described norbornyl group may be a carbonyl carbon.

It is preferable that “Z^{2c}—SO₃—” in Formula (d1-2) is different from the above-described anions represented by Formulae (AN1) to (AN3). For example, Z^{2e} is preferably a group other than an aryl group. For example, in Z²c, atoms at α-position and β-position with respect to —SO₃⁻ are preferably atoms other than carbon atoms having a fluorine atom as a substituent. For example, in Z²c, it is preferable that the atom at α-position and/or the atom at β-position with respect to —SO₃⁻ is a ring member atom in a cyclic group.

In Formula (d1-3), R⁵² represents an organic group (preferably, a hydrocarbon group having a fluorine atom), Y³ represents a linear, branched, or cyclic alkylene group, an arylene group, or a carbonyl group, and Rf represents a hydrocarbon group.

In Formula (d1-4), R⁵³ and R⁵⁴ each independently represent an organic group (preferably, a hydrocarbon group having a fluorine atom). R⁵³ and R⁵⁴ may be bonded to each other to form a ring.

The organic anion may be used alone or in combination of two or more kinds thereof.

Specific examples of Zⁿ⁻ in Formulae (4A) and (4B) are shown below, but the present invention is not limited thereto. The following specific examples are specific examples in which n in Formulae (4A) and (4B) is 1.

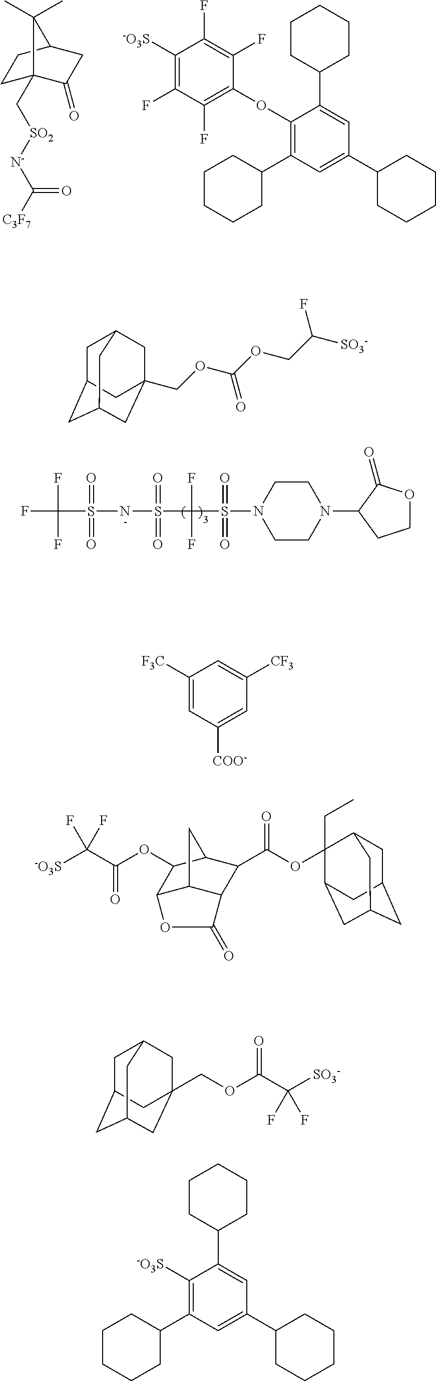

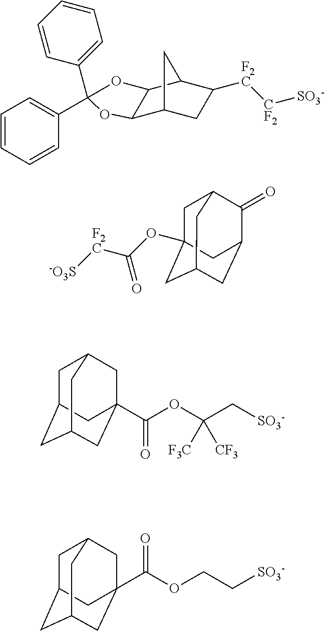

The onium salt compound (A) represented by Formula (4A) or Formula (4B) is also preferably a compound corresponding to at least one selected from the group consisting of the compounds (I) to (II). That is, Zⁿ⁻ in Formula (4A) or (4B) is also preferably a divalent or higher valent anion in the following compounds (I) to (II).

### (Compound (I))

The compound (I) is a compound having one or more of the following structural moieties X and one or more of the following structural moieties Y, in which the compound generates an acid including the following first acidic moiety derived from the following structural moiety X and the following second acidic moiety derived from the following structural moiety Y by irradiation with actinic ray or radiation.

Structural moiety X: a structural moiety which consists of an anionic moiety A₁⁻ and a cationic moiety M₁⁺, and forms a first acidic moiety represented by HA₁ by irradiation with actinic ray or radiation

Structural moiety Y: a structural moiety which consists of an anionic moiety A₂⁻ and a cationic moiety M₂⁺, and forms a second acidic moiety represented by HA₂ by irradiation with actinic ray or radiation

The compound (I) satisfies the following condition I.

Condition I: a compound PI, which is formed by, in the compound (I), replacing the cationic moiety M₁⁺ in the structural moiety X and the cationic moiety M₂⁺ in the structural moiety Y with H⁺, has an acid dissociation constant a1 derived from the acidic moiety represented by HA₁, formed by replacing the cationic moiety M₁⁺ in the structural moiety X with H⁺, and has an acid dissociation constant a2 derived from the acidic moiety represented by HA₂, formed by replacing the cationic moiety M₂⁺ in the structural moiety Y with H⁺, in which the acid dissociation constant a2 is larger than the acid dissociation constant a1

Hereinafter, the condition I will be described in more detail.

In a case where the compound (I) is, for example, a compound that generates an acid having one first acidic moiety derived from the above-described structural moiety X and one second acidic moiety derived from the above-described structural moiety Y, the compound PI corresponds to “compound having HA₁ and HA₂”.

As the acid dissociation constant a1 and the acid dissociation constant a2 of such a compound PI, more specifically, in a case of obtaining acid dissociation constants of the compound PI, a pKa in a case where the compound PI is to be “compound having A₁⁻ and HA₂” is defined as the acid dissociation constant a1, and a pKa in a case where the “compound having A₁⁻ and HA₂” is to be “compound having A₁⁻ and A₂⁻” is defined as the acid dissociation constant a2.

In a case where the compound (I) is, for example, a compound that generates an acid having two first acidic moieties derived from the above-described structural moiety X and one second acidic moiety derived from the above-described structural moiety Y, the compound PI corresponds to “compound having two HA₁'s and one HA₂”.

In a case of obtaining acid dissociation constants of such a compound PI, an acid dissociation constant in a case where the compound PI is to be “compound having one A₁⁻, one HA₁, and one HA₂” and an acid dissociation constant in a case where the “compound having one A₁⁻, one HA₁, and one HA₂” is to be “compound having two A₁⁻ and one HA₂” correspond to the above-described acid dissociation constant a1. An acid dissociation constant in a case where the “compound having two A₁⁻ and one HA₂” is to be “compound having two A₁⁻ and one A₂⁻” corresponds to the acid dissociation constant a2. That is, in the compound PI, in a case of a plurality of acid dissociation constants derived from the acidic moiety represented by HA₁, which is formed by replacing the above-described cationic moiety M₁⁺ in the above-described structural moiety X with H⁺, a value of the acid dissociation constant a2 is larger than the largest value of the plurality of acid dissociation constants a1. In a case where the acid dissociation constant in a case where the compound PI is to be the “compound having one A₁⁻, one HA₁, and one HA₂” is defined as aa, and the acid dissociation constant in a case where the “compound having one A₁⁻, one HA₁, and one HA₂” is to be the “compound having two A₁⁻ and one HA₂” is defined as ab, a relationship between aa and ab satisfies aa<ab.

The acid dissociation constant a1 and the acid dissociation constant a2 can be obtained by the above-described method for measuring an acid dissociation constant.

The above-described compound PI corresponds to an acid generated in a case where the compound (I) is irradiated with actinic ray or radiation.

In a case where the compound (I) has two or more of the structural moieties X, the structural moieties X may be the same or different from each other. In addition, two or more of A₁'s and two or more of M₁⁺ may be the same or different from each other.

In the compound (I), A₁⁻ and A₂⁻, and M₁⁺ and M₂⁺ may be the same or different from each other, but it is preferable that A₁⁻ and A₂⁻ are different from each other.

In the above-described compound PI, a difference (absolute value) between the acid dissociation constant a1 (in a case of a preferably of acid dissociation constants a1, the maximum value thereof) and the acid dissociation constant a2 is preferably 0.1 or more, more preferably 0.5 or more, and still more preferably 1.0 or more. The upper limit value of the difference (absolute value) between the acid dissociation constant a1 (in a case of a preferably of acid dissociation constants a1, the maximum value thereof) and the acid dissociation constant a2 is not particularly limited, but is, for example, 16 or less.

In the above-described compound PI, the acid dissociation constant a2 is, for example, 20 or less, preferably 15 or less. The lower limit value of the acid dissociation constant a2 is preferably −4.0 or more.

In the above-described compound PI, the acid dissociation constant a1 is, for example, 2.0 or less, preferably 0 or less. The lower limit value of the acid dissociation constant a1 is preferably −20.0 or more.

The anionic moiety A₁⁻ and the anionic moiety A₂⁻ are structural moieties including a negatively charged atom or atomic group, and examples thereof include structural moieties selected from the group consisting of Formulae (AA-1) to (AA-3), and Formulae (BB-1) to (BB-6).

The anionic moiety A₁⁻ is preferably an acidic moiety capable of forming an acidic moiety having a small acid dissociation constant, and among these, any one of Formulae (AA-1) to (AA-3) is more preferable and any one of Formula (AA-1) or (AA-3) is still more preferable.

In addition, the anionic moiety A₂⁻ is preferably an acidic moiety capable of forming an acidic moiety having a larger acid dissociation constant than the anionic moiety A₁⁻, and any one of Formulae (BB-1) to (BB-6) is more preferable and any one of Formula (BB-1) or (BB-4) is still more preferable.

In Formulae (AA-1) to (AA-3) and Formulae (BB-1) to (BB-6), *represents a bonding position.

In Formula (AA-2), R^{A} represents a monovalent organic group. The monovalent organic group represented by R^{A} is not particularly limited, and examples thereof include a cyano group, a trifluoromethyl group, and a methanesulfonyl group.

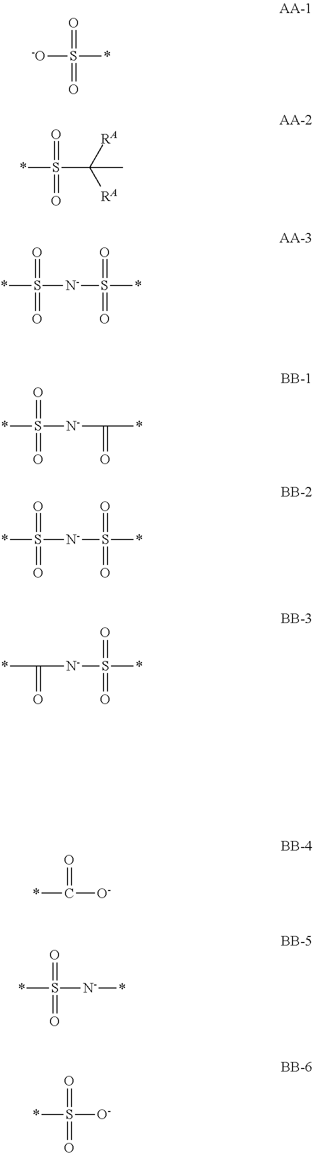

The cationic moiety M₁⁺ and the cationic moiety M₂⁺ are a structural moiety including a positively charged atom or atomic group, and examples thereof include an organic cation having a charge of 1.

In a case where the onium salt compound (A) corresponds to the compound (1), the cationic moiety M₁⁺ and the cationic moiety M₂⁺ correspond to a sulfonium cation in the compound represented by Formula (4A) or an iodonium cation in the compound represented by Formula (4B), and the total number of the cationic moiety M₁⁺ and the cationic moiety M₂⁺ included in the compound corresponds to n in the compound represented by Formula (4A) or (4B).

The cationic moiety M₁⁺ and the cationic moiety M₂⁺ may have the same structure or may not have the same structure, but it is preferable that the cationic moiety M₁⁺ and the cationic moiety M₂⁺ have the same structure.

The specific structure of the onium salt compound (A) in a case of corresponding to the compound (I) is not particularly limited, and examples thereof include compounds represented by Formulae (Ia-1) to (Ia-5) described later.

### —Compound Represented by Formula (Ia-1)—

Hereinafter, first, the compound represented by Formula (Ia-1) will be described.

M₁₁⁺A₁₁⁺-L₁-A₁₂⁻M₁₂⁺ (Ia-1)

The divalent anion represented by A₁₁⁻-L₁-A₁₂ in Formula (Ia-1) corresponds to Zⁿ⁻ in the compound represented by Formula (4A) or (4B), and in this case, n in Formulae (4A) and (4B) is 2.

The compound represented by Formula (Ia-1) generates an acid represented by HA₁₁-L₁-A₁₂H by irradiation with actinic ray or radiation.

In Formula (Ia-1), M₁ₗ⁺ and M₁₂⁺ each independently represent a sulfonium cation in the compound represented by Formula (4A) or an iodonium cation in the compound represented by Formula (4B).

A₁₁⁻ and A₁₂⁻ each independently represent a monovalent anionic functional group.

L₁ represents a divalent linking group.

M₁ₗ⁺ and M₁₂⁺ may be the same or different from each other.

A₁₁⁻ and A₁₂⁻ may be the same or different from each other, but it is preferable to be different from each other.

However, in the compound PIa (HA₁₁-L₁-A₁₂H) formed by replacing cations represented by M₁ₗ⁺ and M₁₂⁺ with H⁺ in Formula (Ia-1), the acid dissociation constant a2 derived from the acidic moiety represented by A₁₂H is larger than the acid dissociation constant a1 derived from the acidic moiety represented by HA₁₁. Suitable values of the acid dissociation constant a1 and the acid dissociation constant a2 are as described above. The acid generated from the compound PIa and the acid generated from the compound represented by Formula (Ia-1) by irradiation with actinic ray or radiation are the same.

In addition, at least one of M₁ₗ⁺, M₁₂⁺, A₁₁⁻, A₁₂⁻, or L₁ may have an acid-decomposable group as a substituent.

In Formula (Ia-1), the sulfonium cation in the compound represented by Formula (4A) represented by M₁₁⁺ and M₁₂⁺ or the iodonium cation in the compound represented by Formula (4B) is as described above, and a suitable aspect thereof is also the same.

The monovalent anionic functional group represented by A₁₁⁻ is intended to be a monovalent group including the above-described anionic moiety A₁⁻. In addition, the monovalent anionic functional group represented by A₁₂⁻ is intended to be a monovalent group including the above-described anionic moiety A₂⁻.

As the monovalent anionic functional group represented by A₁₁⁻ and A₁₂⁻, a monovalent anionic functional group including the anionic moiety of any of Formulae (AA-1) to (AA-3) and Formulae (BB-1) to (BB-6) described above is preferable, and a monovalent anionic functional group selected from the group consisting of Formulae (AX-1) to (AX-3) and Formulae (BX-1) to (BX-7) is more preferable. Among these, as the monovalent anionic functional group represented by A₁₁⁻, a monovalent anionic functional group represented by any of Formulae (AX-1) to (AX-3) is preferable. As the monovalent anionic functional group represented by A₁₂⁻, a monovalent anionic functional group represented by any of Formulae (BX-1) to (BX-7) is preferable, and a monovalent anionic functional group represented by any of Formulae (BX-1) to (BX-6) is more preferable.

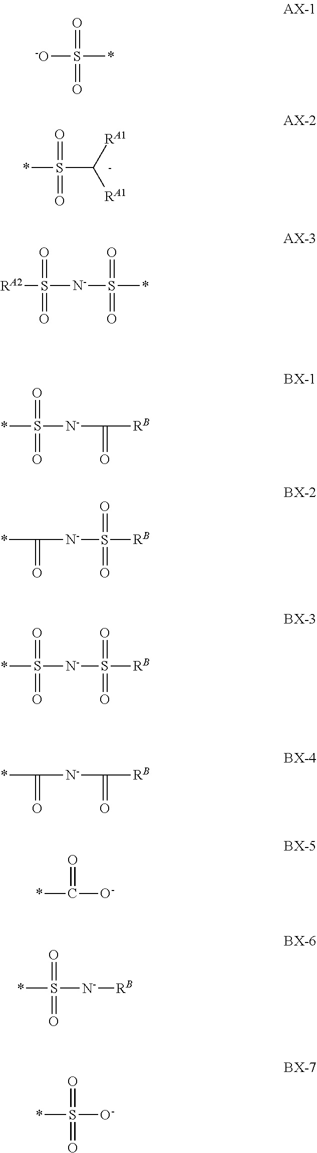

In Formulae (AX-1) to (AX-3), R^{A1} and R^{A2} each independently represent a monovalent organic group. *represents a bonding position.

The monovalent organic group represented by R^{A1} is not particularly limited, and examples thereof include a cyano group, a trifluoromethyl group, and a methanesulfonyl group.

As the monovalent organic group represented by R^{A2}, a linear, branched, or cyclic alkyl group, or an aryl group is preferable.

The number of carbon atoms in the above-described alkyl group is preferably 1 to 15, more preferably 1 to 10, and still more preferably 1 to 6.

The above-described alkyl group may have a substituent. As the substituent, a fluorine atom or a cyano group is preferable, and a fluorine atom is more preferable. In a case where the above-described alkyl group has a fluorine atom as the substituent, the substituent may be a perfluoroalkyl group.

The above-described aryl group is preferably a phenyl group or a naphthyl group, and more preferably a phenyl group.

The above-described aryl group may have a substituent. As the substituent, a fluorine atom, an iodine atom, a perfluoroalkyl group (for example, preferably having 1 to 10 carbon atoms and more preferably having 1 to 6 carbon atoms), or a cyano group is preferable, and a fluorine atom, an iodine atom, or a perfluoroalkyl group is more preferable.

In Formulae (BX-1) to (BX-4) and (BX-6), R^{B} represents a monovalent organic group. *represents a bonding position.

As the monovalent organic group represented by R^{B}, a linear, branched, or cyclic alkyl group, or an aryl group is preferable.

The number of carbon atoms in the above-described alkyl group is preferably 1 to 15, more preferably 1 to 10, and still more preferably 1 to 6.

The above-described alkyl group may have a substituent. The substituent is not particularly limited, but a fluorine atom or a cyano group is preferable, and a fluorine atom is more preferable. In a case where the above-described alkyl group has a fluorine atom as the substituent, the substituent may be a perfluoroalkyl group.

In a case where the carbon atom that is a bonding position in the alkyl group has a substituent, the substituent is preferably a substituent other than a fluorine atom or a cyano group. Here, the carbon atom that serves as a bonding position in the alkyl group corresponds to, for example, in a case of Formulae (BX-1) and (BX-4), a carbon atom that is directly bonded to —CO— specified in the formula of the alkyl group, in a case of Formulae (BX-2) and (BX-3), a carbon atom that is directly bonded to —SO₂— specified in the formula of the alkyl group, and in a case of Formula (BX-6), a carbon atom that is directly bonded to N⁻ specified in the formula of the alkyl group.

The above-described alkyl group may have a carbon atom substituted with a carbonyl carbon.

The above-described aryl group is preferably a phenyl group or a naphthyl group, and more preferably a phenyl group.

The above-described aryl group may have a substituent. As the substituent, a fluorine atom, an iodine atom, a perfluoroalkyl group (for example, preferably having 1 to 10 carbon atoms and more preferably having 1 to 6 carbon atoms), a cyano group, an alkyl group (for example, preferably having 1 to 10 carbon atoms and more preferably having 1 to 6 carbon atoms), an alkoxy group (for example, preferably having 1 to 10 carbon atoms and more preferably having 1 to 6 carbon atoms), or an alkoxycarbonyl group (for example, preferably having 2 to 10 carbon atoms and more preferably having 2 to 6 carbon atoms) is preferable; and a fluorine atom, an iodine atom, a perfluoroalkyl group, an alkyl group, an alkoxy group, or an alkoxycarbonyl group is more preferable.

In Formula (Ia-1), the divalent liking group represented by L₁ is not particularly limited, and examples thereof include —CO—, —NR—, —O—, —S—, —SO—, —SO₂—, an alkylene group (preferably having 1 to 6 carbon atoms; may be linear or branched), a cycloalkylene group (preferably having 3 to 15 carbon atoms), an alkenylene group (preferably having 2 to 6 carbon atoms), a divalent aliphatic heterocyclic group (preferably a 5- to 10-membered ring, more preferably a 5- to 7-membered ring, and still more preferably a 5- or 6-membered ring; each having at least one of an N atom, an O atom, an S atom, or an Se atom in the ring structure), and a divalent aromatic heterocyclic group (preferably a 5- to 10-membered ring, more preferably a 5- to 7-membered ring, and still more preferably a 5- or 6-membered ring; each having at least one of an N atom, an O atom, an S atom, or an Se atom in the ring structure), a divalent aromatic hydrocarbon ring group (preferably a 6- to 10-membered ring, and more preferably a 6-membered ring), and a divalent linking group formed by a combination of a plurality of these groups. Examples of R include a hydrogen atom and a monovalent organic group. The monovalent organic group is not particularly limited, but is preferably, for example, an alkyl group (preferably having 1 to 6 carbon atoms).

The above-described alkylene group, the above-described cycloalkylene group, the above-described alkenylene group, and the above-described divalent aliphatic heterocyclic group, divalent aromatic heterocyclic group, and divalent aromatic hydrocarbon ring group may have a substituent. Examples of the substituent include a halogen atom (preferably, a fluorine atom).

Among these, as the divalent linking group represented by L₁, a divalent linking group represented by Formula (L1) is preferable.

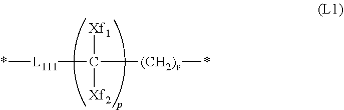

In Formula (L1), L₁₁₁ represents a single bond or a divalent linking group.

The divalent linking group represented by L₁₁₁ is not particularly limited, and examples thereof include —CO—, —NH—, —O—, —SO—, —SO₂—, and an alkylene group which may have a substituent (preferably having 1 to 6 carbon atoms; may be linear or branched), a cycloalkylene group (preferably having 3 to 15 carbon atoms) which may have a substituent, an aryl group (preferably having 6 to 10 carbon atoms) which may have a substituent, and a divalent linking group obtained by combining a plurality of these groups. The substituent is not particularly limited, and examples thereof include a halogen atom.

p represents an integer of 0 to 3, and preferably represents an integer of 1 to 3.

v represents an integer of 0 or 1.

Xf₁'s each independently represent a fluorine atom or an alkyl group substituted with at least one fluorine atom. The number of carbon atoms in the alkyl group is preferably 1 to 10 and more preferably 1 to 4. The alkyl group substituted with at least one fluorine atom is preferably a perfluoroalkyl group.

Xf₂'s each independently represent a hydrogen atom, an alkyl group which may have a fluorine atom as a substituent, or a fluorine atom. The number of carbon atoms in the alkyl group is preferably 1 to 10 and more preferably 1 to 4. Among these, Xf₂ preferably represents a fluorine atom or an alkyl group substituted with at least one fluorine atom, and more preferably represents a fluorine atom or a perfluoroalkyl group.

Among these, as each of Xf₁ and Xf₂, a fluorine atom or a perfluoroalkyl group having 1 to 4 carbon atoms is preferable, and a fluorine atom or CF₃ is more preferable. In particular, it is still more preferable that both Xf₁ and Xf₂ are fluorine atoms. *represents a bonding position.

In a case where L₁₁ in Formula (Ia-1) represents the divalent linking group represented by Formula (L1), it is preferable that the bonding site (*) on the L₁₁₁ side of Formula (L1) is bonded to A₁₂⁻ of Formula (Ia-1).

### —Compounds Represented by Formulae (Ia-2) to (Ia-4)—

Next, the compounds represented by Formulae (Ia-2) to (Ia-4) will be described.

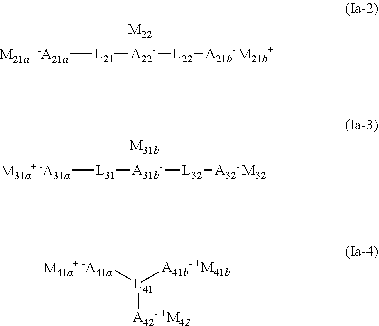

The trivalent anion in Formulae (Ia-2) to (Ia-4) corresponds to Zⁿ⁻ in the compound represented by Formula (4A) or (4B), and in this case, n in Formulae (4A) and (4B) is 3.

In Formula (Ta-2), A₂₁ₐ⁻ and A_{21b}⁻ each independently represent a monovalent anionic functional group. Here, the monovalent anionic functional group represented by A₂₁ₐ⁻ and A_{21b}⁻ is intended to be a monovalent group including the above-described anionic moiety A₁⁻. The monovalent anionic functional group represented by A₂₁ₐ⁻ and A_{21b}⁻ is not particularly limited, and examples thereof include the monovalent anionic functional group selected from the group consisting of Formulae (AX-1) to (AX-3) described above.

A₂₂⁻ represents a divalent anionic functional group. Here, the divalent anionic functional group represented by A₂₂⁻ is intended to be a divalent linking group including the above-described anionic moiety A₂⁻. Examples of the divalent anionic functional group represented by A₂₂⁻ include a divalent anionic functional group represented by Formulae (BX-8) to (BX-11).

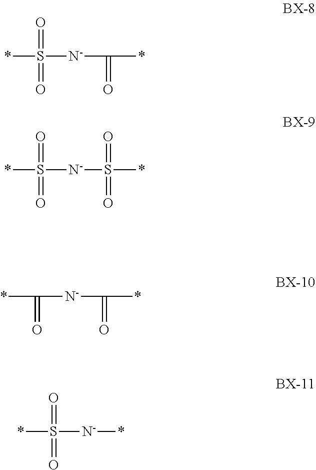 [SMILES: *C(=O)[N-]S(*)(=O)=O ; *S(=O)(=O)[N-]S(*)(=O)=O ; *C(=O)[N-]C(*)=O ; *[N-]S(*)(=O)=O]

M₂₁ₐ⁺, M_{21b}⁺, and M₂₂⁺ each independently represent a sulfonium cation in the compound represented by Formula (4A) or an iodonium cation in the compound represented by Formula (4B), and suitable aspects thereof are also the same.

L₂₁ and L₂₂ each independently represent a divalent organic group.

In a compound PIa-2 of Formula (Ia-2), in which the organic cation represented by M₂₁ₐ⁺, M_{21b}⁺, and M₂₂⁺ is replaced with H⁺, an acid dissociation constant a2 derived from an acidic moiety represented by A₂₂H is larger than an acid dissociation constant a1-1 derived from A₂₁ₐH and an acid dissociation constant a1-2 derived from an acidic moiety represented by A_{21b}H. The acid dissociation constant a1-1 and the acid dissociation constant a1-2 correspond to the above-described acid dissociation constant a1.

A₂₁ₐ⁻ and A_{21b}⁻ may be the same or different from each other. M₂₁ₐ⁺, M_{21b}⁺, and M₂₂⁺ may be the same or different from each other.

At least one of M₂₁ₐ⁺, M_{21b}⁺, M₂₂⁺, A₂₁ₐ⁻, A_{21b}⁻, L₂₁, or L₂₂ may have an acid-decomposable group as a substituent.

In Formula (Ia-3), A₃₁ₐ⁻ and A₃₂ₐ⁻ each independently represent a monovalent anionic functional group. The definition of the monovalent anionic functional group represented by A₃₁ₐ⁻ is the same as A₂₁ₐ⁻ and A₂₁⁻ in Formula (Ia-2), and a suitable aspect thereof is also the same.

The monovalent anionic functional group represented by A₃₂⁻ is intended to be a monovalent group including the above-described anionic moiety A₂⁻. The monovalent anionic functional group represented by A₃₂⁻ is not particularly limited, and examples thereof include the monovalent anionic functional group selected from the group consisting of Formulae (BX-1) to (BX-7) described above.

A_{31b}⁻ represents a divalent anionic functional group. Here, the divalent anionic functional group represented by A_{31b}⁻ is intended to be a divalent group including the above-described anionic moiety A₁⁻. Examples of the divalent anionic functional group represented by A_{31b}⁻ include a divalent anionic functional group represented by Formula (AX-4).

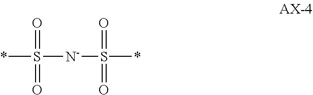 [SMILES: *S(=O)(=O)[N-]S(*)(=O)=O]

M₃₁ₐ⁺, M_{31b}⁺, and M₃₂⁺ each independently represent a sulfonium cation in the compound represented by Formula (4A) or an iodonium cation in the compound represented by Formula (4B), and suitable aspects thereof are also the same.

L₃₁ and L₃₂ each independently represent a divalent organic group.

In a compound PIa-3 of Formula (Ia-3), in which the organic cation represented by M₃₁ₐ⁺, M_{31b}⁺, and M₃₂⁺ is replaced with H⁺, an acid dissociation constant a2 derived from an acidic moiety represented by A₃₂H is larger than an acid dissociation constant a1-3 derived from an acidic moiety represented by A₃₁ₐH and an acid dissociation constant a1-4 derived from an acidic moiety represented by A_{31b}H. The acid dissociation constant a1-3 and the acid dissociation constant a1-4 correspond to the above-described acid dissociation constant a1.

A₃₁ₐ⁻ and A₃₂⁻ may be the same or different from each other. In addition, M₃₁ₐ⁺, M_{31b}⁺, and M₃₂⁺ may be the same or different from each other.

At least one of M₃₁ₐ⁺, M_{31b}⁺, M₃₂⁺, A₃₁ₐ⁻, A₃₂⁻, L₃₁, or L₃₂ may have an acid-decomposable group as a substituent.

In Formula (Ia-4), A₄₁ₐ⁻, A_{41b}⁻, and A₄₂⁻ each independently represent a monovalent anionic functional group. The definition of the monovalent anionic functional group represented by A₄₁ₐ⁻ and A₄₁⁻ is the same as A₂₁ₐ⁻ and A₂₁⁻ in Formula (Ia-2). The definition of the monovalent anionic functional group represented by A₄₂⁻ is the same as A₃₂⁻ in Formula (Ia-3), and a suitable aspect thereof is also the same.

M₄₁ₐ⁺, M_{41b}⁺, and M₄₂⁺ each independently represent a sulfonium cation in the compound represented by Formula (4A) or an iodonium cation in the compound represented by Formula (4B), and suitable aspects thereof are also the same.

L₄₁ represents a trivalent organic group.

In a compound PIa-4 of Formula (Ia-4), in which the organic cation represented by M₄₁ₐ⁺, M_{41b}⁺, and M₄₂⁺ is replaced with H⁺, an acid dissociation constant a2 derived from an acidic moiety represented by A₄₂H is larger than an acid dissociation constant a1-5 derived from an acidic moiety represented by A₄₁ₐH and an acid dissociation constant a1-6 derived from an acidic moiety represented by A_{41b}H. The acid dissociation constant a1-5 and the acid dissociation constant a1-6 correspond to the above-described acid dissociation constant a1.

A₄₁ₐ⁻, A_{41b}⁻, and A₄₂⁻ may be the same or different from each other. In addition, M₄₁ₐ⁺, M_{41b}⁺, and M₄₂⁺ may be the same or different from each other.

At least one of M₄₁ₐ⁺, M_{41b}⁺, M₄₂⁺, A₄₁ₐ⁻, A_{41b}⁻, A₄₂⁻, or L₄₁ may have an acid-decomposable group as a substituent.

The divalent organic group represented by L₂₁ and L₂₂ in Formula (Ia-2) and L₃₁ and L₃₂ in Formula (Ia-3) is not particularly limited, and examples thereof include —CO—, —NR—, —O—, —S—, —SO—, —SO₂—, an alkylene group (preferably having 1 to 6 carbon atoms; may be linear or branched), a cycloalkylene group (preferably having 3 to 15 carbon atoms), an alkenylene group (preferably having 2 to 6 carbon atoms), a divalent aliphatic heterocyclic group (preferably a 5- to 10-membered ring, more preferably a 5- to 7-membered ring, and still more preferably a 5- or 6-membered ring; each having at least one of an N atom, an O atom, an S atom, or an Se atom in the ring structure), and a divalent aromatic heterocyclic group (preferably a 5- to 10-membered ring, more preferably a 5- to 7-membered ring, and still more preferably a 5- or 6-membered ring; each having at least one of an N atom, an O atom, an S atom, or an Se atom in the ring structure), a divalent aromatic hydrocarbon ring group (preferably a 6- to 10-membered ring, and more preferably a 6-membered ring), and a divalent organic group formed by a combination of a plurality of these groups. Examples of R in —NR— include a hydrogen atom and a monovalent organic group. The monovalent organic group is not particularly limited, but is preferably, for example, an alkyl group (preferably having 1 to 6 carbon atoms).

The above-described alkylene group, the above-described cycloalkylene group, the above-described alkenylene group, and the above-described divalent aliphatic heterocyclic group, divalent aromatic heterocyclic group, and divalent aromatic hydrocarbon ring group may have a substituent. Examples of the substituent include a halogen atom (preferably, a fluorine atom).

As the divalent organic group represented by L₂₁ and L₂₂ in Formula (Ia-2) and L₃₁ and L₃₂ in Formula (Ia-3), for example, a divalent organic group represented by Formula (L2) is also preferable.

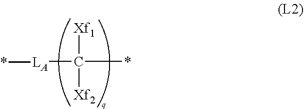

In Formula (L2), q represents an integer of 1 to 3. *represents a bonding position.

Xf's each independently represent a fluorine atom or an alkyl group substituted with at least one fluorine atom. The number of carbon atoms in the alkyl group is preferably 1 to 10 and more preferably 1 to 4. The alkyl group substituted with at least one fluorine atom is preferably a perfluoroalkyl group.

Xf is preferably a fluorine atom or a perfluoroalkyl group having 1 to 4 carbon atoms, and more preferably a fluorine atom or CF₃. In particular, it is still more preferable that both Xf's are fluorine atoms.

L_{A} represents a single bond or a divalent linking group.

The divalent linking group represented by L_{A} is not particularly limited, and examples thereof include —CO—, —O—, —SO—, —SO₂—, an alkylene group (preferably having 1 to 6 carbon atoms; may be linear or branched), a cycloalkylene group (preferably having 3 to 15 carbon atoms), a divalent aromatic hydrocarbon ring group (preferably a 6- to 10-membered ring, and more preferably a 6-membered ring), and a divalent linking group formed by a combination of a plurality of these groups.

The above-described alkylene group, the above-described cycloalkylene group, and the divalent aromatic hydrocarbon ring group may have a substituent. Examples of the substituent include a halogen atom (preferably, a fluorine atom).

Examples of the divalent organic group represented by Formula (L2) include *—CF₂—*, *—CF₂—CF₂—*, *—CF₂—CF₂—CF₂—*, *-Ph-O—SO₂—CF₂—*, *-Ph-O—SO₂—CF₂—CF₂—*, *-Ph-O—SO₂—CF₂—CF₂—CF₂—*, and *-Ph-OCO—CF₂—*. Ph is a phenylene group which may have a substituent, and is preferably a 1,4-phenylene group. The substituent is not particularly limited, and an alkyl group (for example, preferably having 1 to 10 carbon atoms and more preferably having 1 to 6 carbon atoms) or an alkoxy group (for example, preferably having 1 to 10 carbon atoms and more preferably having 1 to 6 carbon atoms), or an alkoxycarbonyl group (for example, preferably having 2 to 10 carbon atoms and more preferably having 2 to 6 carbon atoms) is preferable.

In a case where L₂₁ and L₂₂ in Formula (Ia-2) represents the divalent organic group represented by Formula (L2), it is preferable that the bonding site (*) on the L_{A} side of Formula (L2) is bonded to A₂₁ₐ⁻ and A_{21b}⁻ of Formula (Ia-2).

In a case where L₃₁ and L₃₂ in Formula (Ia-3) represents the divalent organic group represented by Formula (L2), it is preferable that the bonding site (*) on the L_{A} side of Formula (L2) is bonded to A₃₁ₐ⁻ and A32⁻ of Formula (Ia-3).

### —Compound Represented by Formula (Ia-5)—

Next, Formula (Ia-5) will be described.

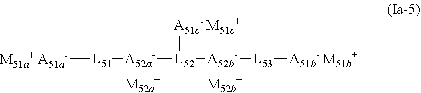

The pentavalent anion in Formula (Ia-5) corresponds to Zⁿ⁻ in the compound represented by Formula (4A) or (4B), and in this case, n in Formulae (4A) and (4B) is 5.

In Formula (Ia-5), A₅₁ₐ⁻, A_{51b}⁻, and A_{51c}⁻ each independently represent a monovalent anionic functional group. Here, the monovalent anionic functional group represented by A₅₁ₐ⁻, A_{51b}⁻, and A_{51c}⁻ is intended to be a monovalent group including the above-described anionic moiety A₁⁻. The monovalent anionic functional group represented by A₅₁ₐ⁻, A_{51b}⁻, and A_{51c}⁻ is not particularly limited, and examples thereof include the monovalent anionic functional group selected from the group consisting of Formulae (AX-1) to (AX-3) described above.

A₅₂ₐ⁻ and A_{52b}⁻ represents a divalent anionic functional group. Here, the divalent anionic functional group represented by A₅₂ₐ⁻ and A_{52b}⁻ is intended to be a divalent group including the above-described anionic moiety A₂⁻. Examples of the divalent anionic functional group represented by A₂₂⁻ include the divalent anionic functional group selected from the group consisting of Formulae (BX-8) to (BX-11) described above.

M₅₁ₐ⁺, M_{51b}⁺, M_{51c}⁺, M₅₂ₐ⁺, and M_{52b}⁺ each independently represent a sulfonium cation in the compound represented by Formula (4A) or an iodonium cation in the compound represented by Formula (4B), and suitable aspects thereof are also the same.

L₅₁ and L₅₃ each independently represent a divalent organic group. The divalent organic group represented by L₅₁ and L₅₃ has the same meaning as L₂₁ and L₂₂ in Formula (Ia-2) described above, and a suitable aspect thereof is also the same.

L₅₂ represents a trivalent organic group. The trivalent organic group represented by L₅₂ has the same meaning as L₄₁ in Formula (Ia-4) described above, and a suitable aspect thereof is also the same.

In a compound PIa-5 of Formula (Ia-5), in which the organic cation represented by M₅₁ₐ⁺, M_{51b}⁺, M_{51c}⁺, M₅₂ₐ⁺, and M_{52b}⁺ is replaced with H⁺, an acid dissociation constant a2-1 derived from an acidic moiety represented by A₅₂ₐH and an acid dissociation constant a2-2 derived from an acidic moiety represented by A_{52b}H are larger than an acid dissociation constant a1-1 derived from an acidic moiety represented by A₅₁ₐH, an acid dissociation constant a1-2 derived from an acidic moiety represented by A_{51b}H, and an acid dissociation constant a1-3 derived from an acidic moiety represented by A_{51c}H. The acid dissociation constants a1-1 to a1-3 correspond to the above-described acid dissociation constant a1, and the acid dissociation constants a2-1 and a2-2 correspond to the above-described acid dissociation constant a2.

A₅₁ₐ⁻, A_{51b}⁻, and A_{51c}⁻ may be the same or different from each other. In addition, A₅₂ₐ⁻ and A_{52b}⁻ may be the same or different from each other.

M₅₁ₐ⁺, M_{51b}⁺, M_{51c}⁺, M₅₂ₐ⁺, and M_{52b}⁺ may be the same or different from each other.

At least one of M_{51b}⁺, M_{51c}⁺, M₅₂ₐ⁺, M_{52b}⁺, A₅₁ₐ⁻, A_{51b}⁻, A_{51c}⁻, L₅₁, L₅₂, or L₅₃ may have an acid-decomposable group as a substituent.

### (Compound (II))

The compound (II) is a compound having two or more of the structural moieties X and one or more of the following structural moieties Z, in which the compound generates an acid including two or more of the above-described first acidic moieties derived from the above-described structural moiety X and a structural moiety Z by irradiation with actinic ray or radiation.

Structural moiety Z: a non-ionic moiety capable of neutralizing an acid

In the compound (II), the definition of the structural moiety X and the definitions of A₁⁻ and M₁⁺ are the same as the definition of the structural moiety X and the definitions of A₁⁻ and M₁⁺ in the above-described compound (I), and suitable aspects thereof are also the same.

In a compound PII formed by replacing the above-described cationic moiety M₁⁺ in the above-described structural moiety X with H⁺ in the above-described compound (II), a suitable range of an acid dissociation constant a1 derived from the acidic moiety represented by HA₁, formed by replacing the above-described cationic moiety M₁⁺ in the above-described structural moiety X with H⁺, is the same as in the acid dissociation constant a1 of the above-described compound PI.

In a case where the compound (II) is, for example, a compound which generates an acid having two of the above-described first acidic moieties derived from the above-described structural moiety X and the above-described structural moiety Z, the compound PII corresponds to “compound having two HA₁”. In a case of obtaining acid dissociation constants of the compound PII, an acid dissociation constant in a case where the compound PII is to be “compound having one A₁⁻ and one HA₁” and an acid dissociation constant in a case where the “compound having one A₁⁻ and one HA₁” is to be “compound having two A₁⁻” correspond to the acid dissociation constant a1.

The acid dissociation constant a1 can be obtained by the above-described method for measuring an acid dissociation constant.

The above-described compound PII corresponds to an acid generated in a case where the compound (TI) is irradiated with actinic ray or radiation.

The above-described two or more of the structural moieties X may be the same or different from each other. Two or more of A₁⁻'s and two or more of M₁⁺ may be the same or different from each other.

The non-ionic moiety capable of neutralizing an acid in the structural moiety Z is not particularly limited, and is preferably, for example, a moiety including a functional group having a group or an electron which is capable of electrostatically interacting with a proton.

Examples of the functional group having a group or electron capable of electrostatically interacting with a proton include a functional group with a macrocyclic structure, such as a cyclic polyether, or a functional group having a nitrogen atom having an unshared electron pair not contributing to π-conjugation. For example, the nitrogen atom having the unshared electron pair, which does not contribute to the π-conjugation, is a nitrogen atom having a partial structure represented by the following formula.

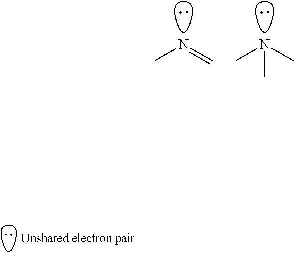

Examples of the partial structure of the functional group having a group or electron which is capable of electrostatically interacting with a proton include a crown ether structure, an azacrown ether structure, primary to tertiary amine structures, a pyridine structure, an imidazole structure, and a pyrazine structure, and among these, primary to tertiary amine structures are preferable.

The specific structure of the onium salt compound (A) in a case of corresponding to the compound (II) is not particularly limited, and examples thereof include a compound represented by Formula (IIa-1) and a compound represented by Formula (IIa-2).

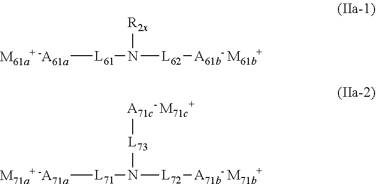

The divalent anion in Formula (IIa-1) corresponds to Zⁿ⁻ in the compound represented by Formula (4A) or (4B), and in this case, n in Formulae (4A) and (4B) is 2. In addition, the trivalent anion in Formula (IIa-2) corresponds to Zⁿ⁻ in the compound represented by Formula (4A) or (4B), and in this case, n in Formulae (4A) and (4B) is 3.

In Formula (IIa-1), A₆₁ₐ⁻ and A_{61b}⁻ have the same meaning as A₁₁⁻ in Formula (ha-1) described above, and suitable aspects thereof are also the same. In addition, M₆₁ₐ⁺ and M_{61b}⁺ each independently represent a sulfonium cation in the compound represented by Formula (4A) or an iodonium cation in the compound represented by Formula (4B), and suitable aspects thereof are also the same.

In Formula (IIa-1), L₆₁ and L₆₂ have the same meaning as L₁ in Formula (Ia-1) described above, and suitable aspects thereof are also the same.

In Formula (IIa-1), R₂ₓ represents a monovalent organic group. The monovalent organic group represented by R₂ₓ is not particularly limited, and examples thereof include an alkyl group (preferably having 1 to 10 carbon atoms; may be linear or branched), a cycloalkyl group (preferably having 3 to 15 carbon atoms), and an alkenyl group (preferably having 2 to 6 carbon atoms). —CH₂— in the alkyl group, the cycloalkyl group, and the alkenyl group in the monovalent organic group represented by R₂ₓ may be substituted with one or a combination of two or more selected from the group consisting of —CO—, —NH—, —O—, —S—, —SO—, and —SO₂—.

The above-described alkylene group, the above-described cycloalkylene group, and the above-described alkenylene group may have a substituent. The substituent is not particularly limited, and examples thereof include a halogen atom (preferably, a fluorine atom).

In a compound PIIa-1 of Formula (IIa-1), in which the organic cation represented by M₆₁ₐ⁺ and M_{61b}⁺ is replaced with H⁺, an acid dissociation constant a1-7 derived from an acidic moiety represented by A₆₁ₐH and an acid dissociation constant a1-8 derived from an acidic moiety represented by A_{61b}H correspond to the above-described acid dissociation constant a1.

The compound PIIa-1 formed by replacing the above-described cationic moieties M₆₁ₐ⁺ and M_{61b}⁺ in the above-described structural moiety X with H⁺ in Formula (IIa-1) corresponds to HA₆₁ₐ-L₆₁-N(R₂ₓ)-L₆₂-A_{61b}H. In addition, the acid generated from the compound PIIa-1 and the acid generated from the compound represented by Formula (IIa-1) by irradiation with actinic ray or radiation are the same.

At least one of M₆₁ₐ⁺, M_{61b}⁺, A₆₁ₐ⁻, A_{61b}⁻, L₆₁, L₆₂, or R₂ₓ may have an acid-decomposable group as a substituent.

In Formula (IIa-2), A₇₁ₐ⁻, A_{71b}⁻, and A_{71c}⁻ have the same meaning as A₁₁⁻ in Formula (Ia-1) described above, and suitable aspects thereof are also the same. M₇₁ₐ⁺, M_{71b}⁺, and M_{71c}⁺ each independently represent a sulfonium cation in the compound represented by Formula (4A) or an iodonium cation in the compound represented by Formula (4B), and suitable aspects thereof are also the same.

In Formula (IIa-2), L₇₁, L₇₂, and L₇₃ have the same meaning as L₁ in Formula (Ia-1) described above, and suitable aspects thereof are also the same.

In a compound PIIa-2 of Formula (IIa-2), in which the organic cation represented by M₇₁ₐ⁺, M_{71b}⁺, and M_{71c}⁺ is replaced with H⁺, an acid dissociation constant a1-9 derived from an acidic moiety represented by A₇₁ₐH, an acid dissociation constant a1-10 derived from an acidic moiety represented by A_{71b}H, and an acid dissociation constant a1-11 derived from an acidic moiety represented by A_{71c}H correspond to the above-described acid dissociation constant a1.

The compound PIIa-2 formed by replacing the above-described cationic moieties M₇₁ₐ⁺, M_{71b}⁺, and M_{71c}⁺ in the above-described structural moiety X with H⁺ in Formula (IIa-2) corresponds to HA₇₁ₐ-L₇₁-N(L₇₃-A_{71c}H)-L₇₂-A_{71b}H. In addition, the acid generated from the compound PIIa-2 and the acid generated from the compound represented by Formula (IIa-2) by irradiation with actinic ray or radiation are the same.

At least one of M₇₁ₐ⁺, M_{71b}⁺, M_{71c}⁺, A₇₁ₐ⁻, A_{71b}⁻, A_{71c}⁻, L₇₁, L₇₂, or L₇₃ may have an acid-decomposable group as a substituent.

Among the specific examples of Zⁿ⁻ in Formulae (4A) and (4B), specific examples in which n is 2 or more are shown below, but the present invention is not limited thereto.

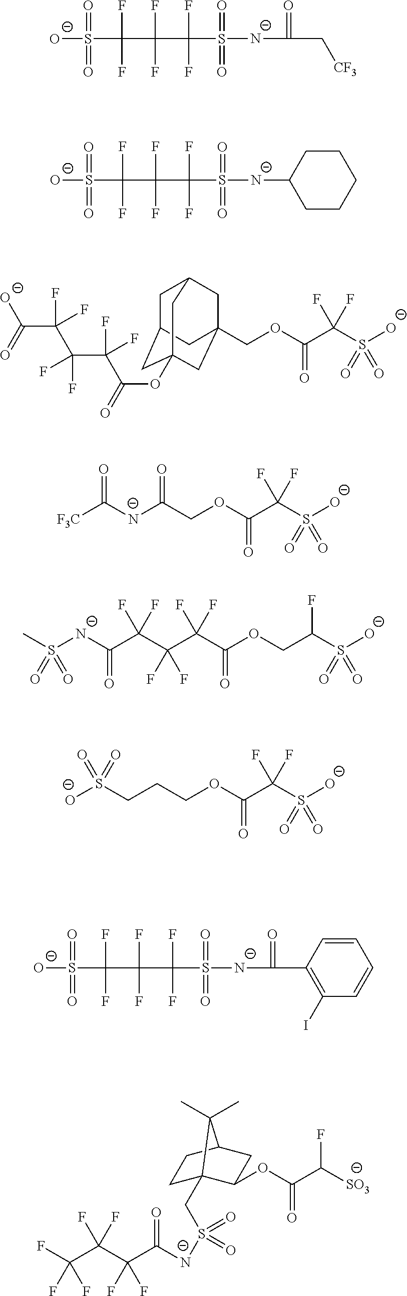

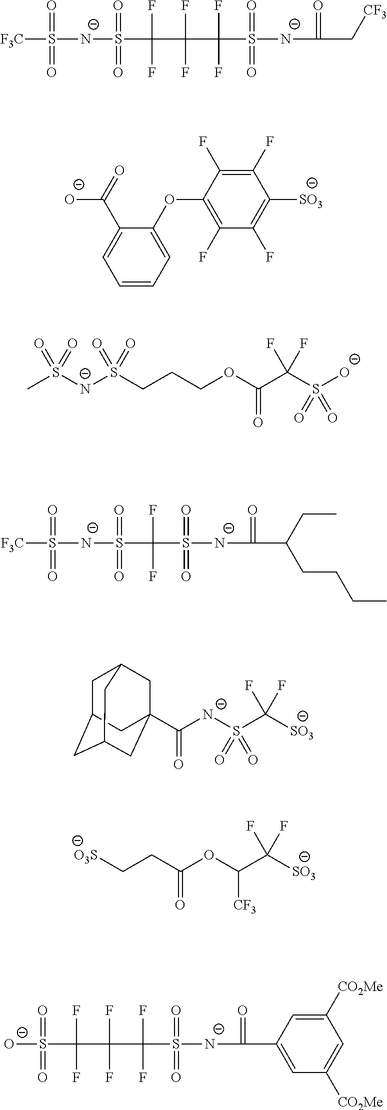

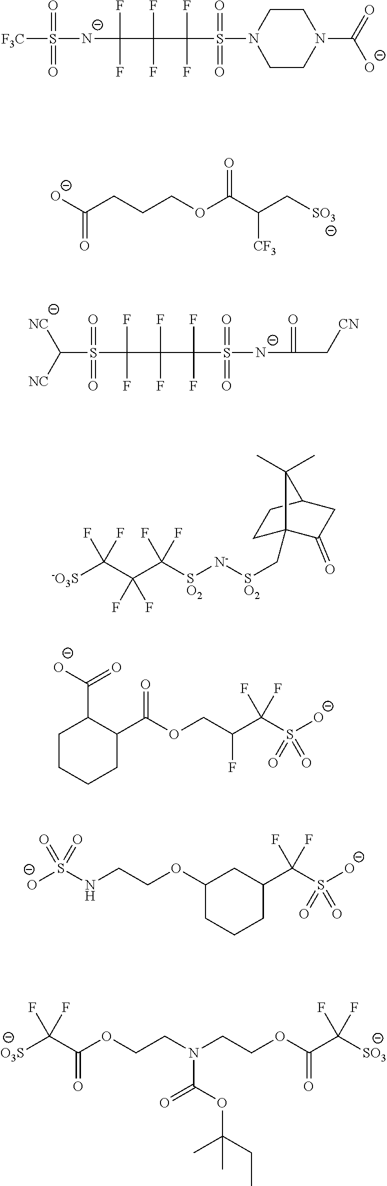

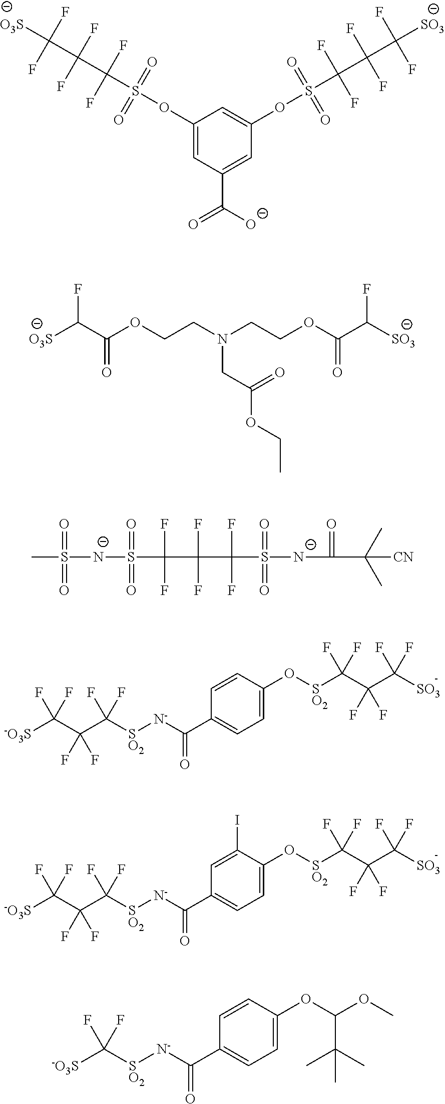

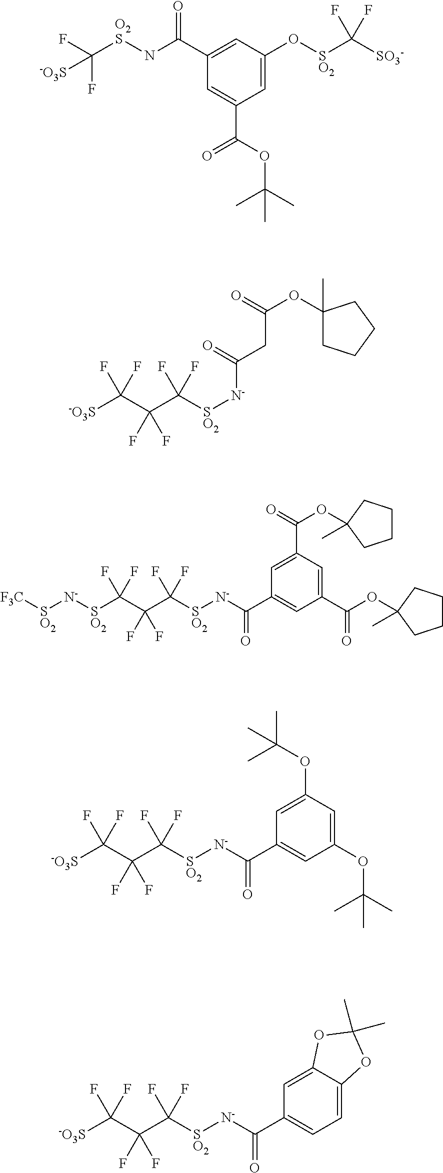

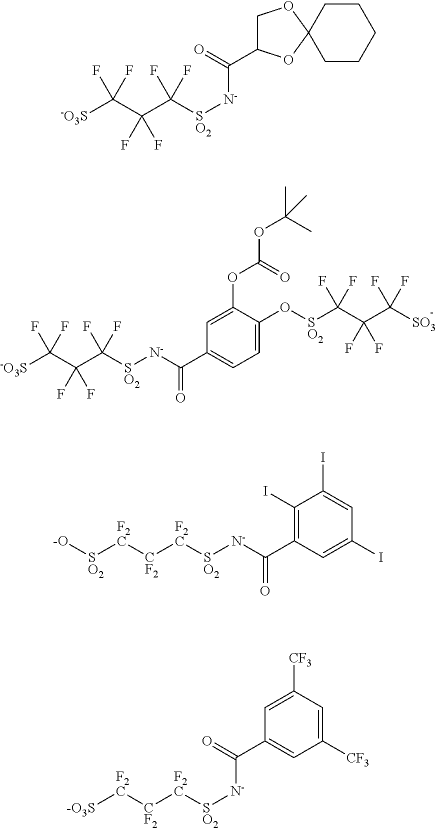

Specific examples of the onium salt compound (A) are shown below, but the present invention is not limited thereto.

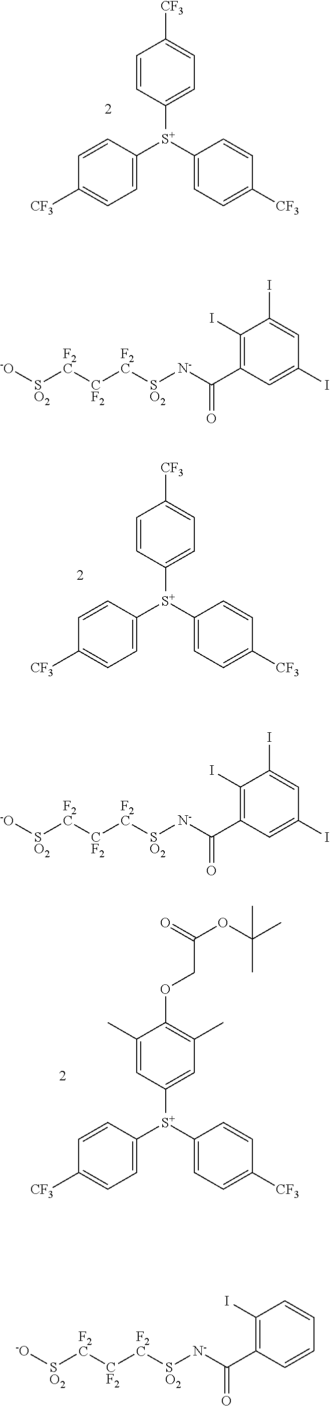

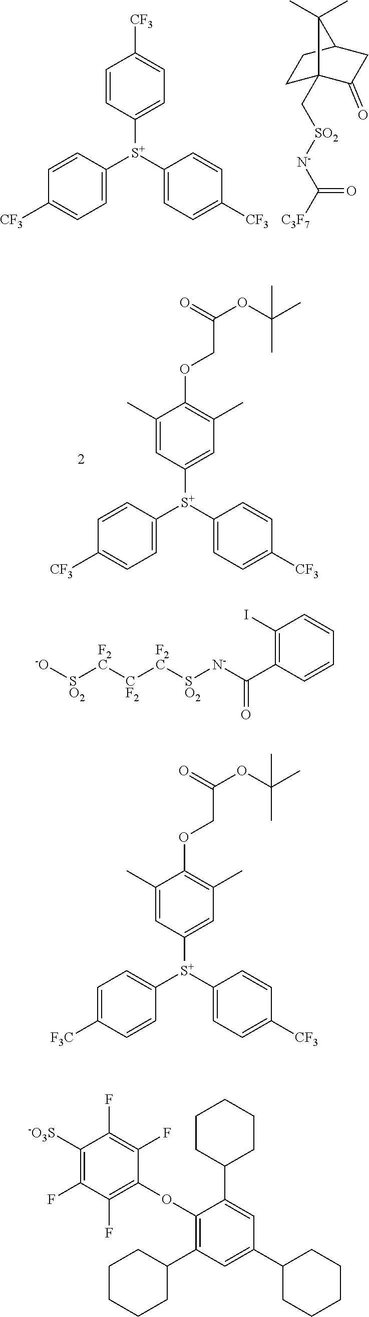

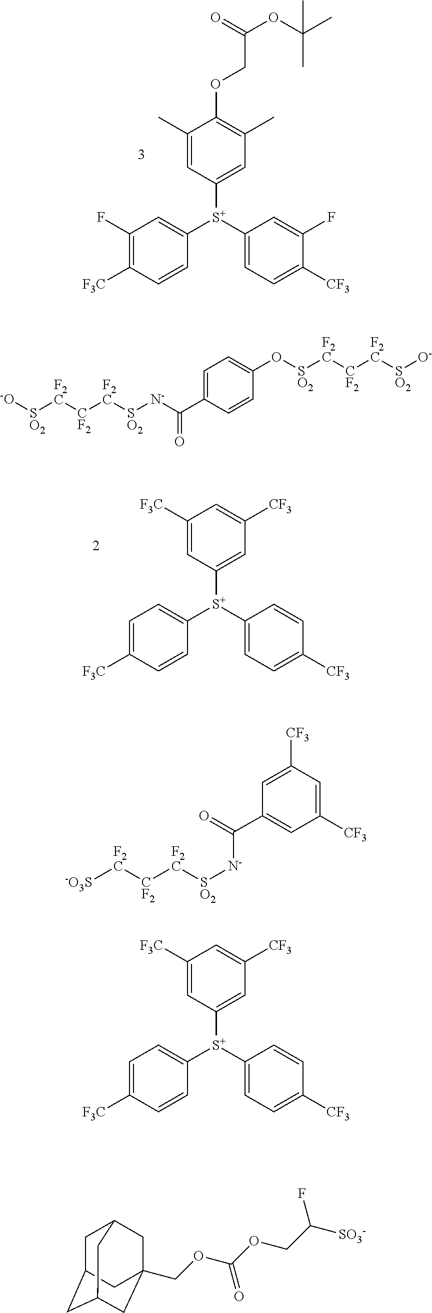

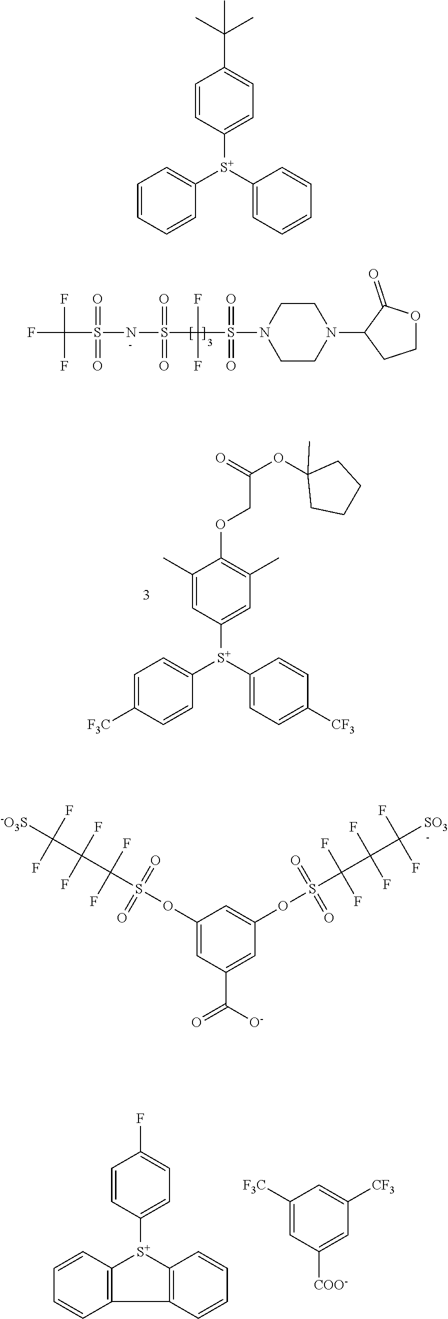

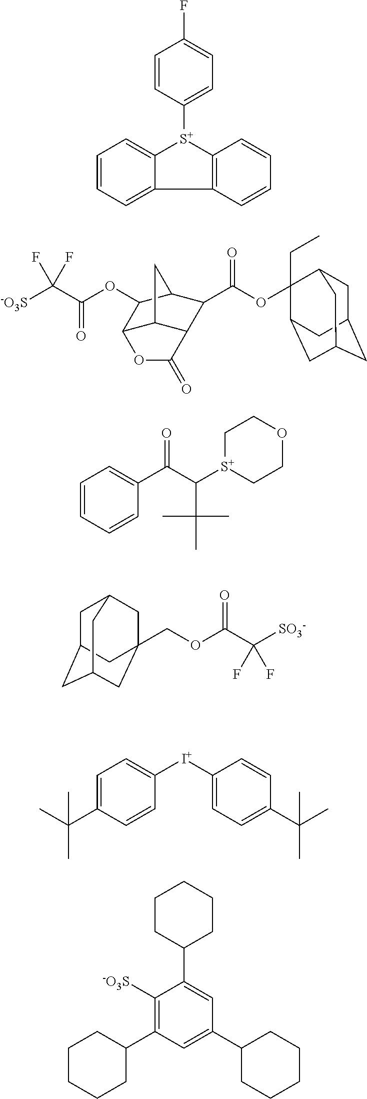

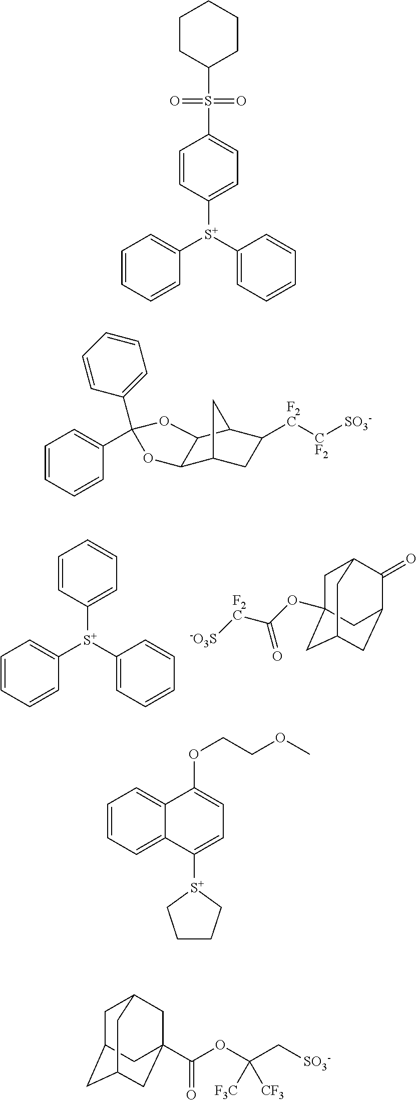

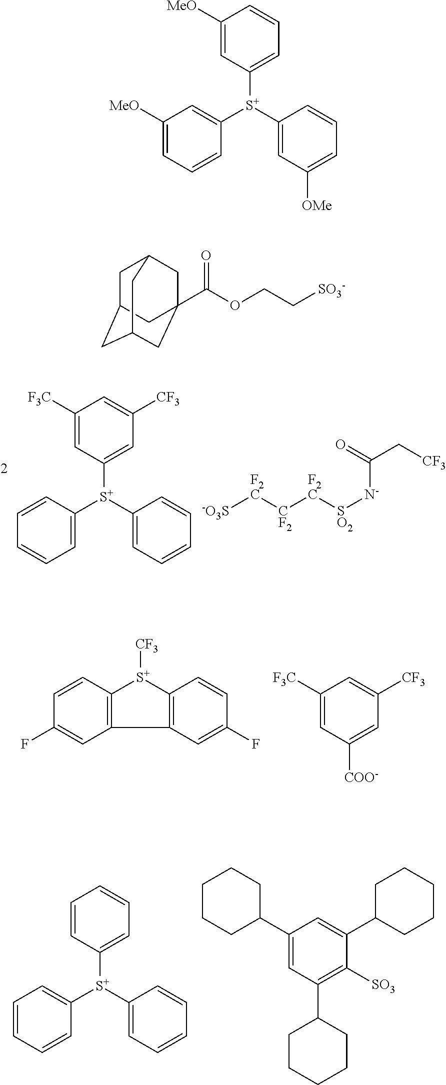

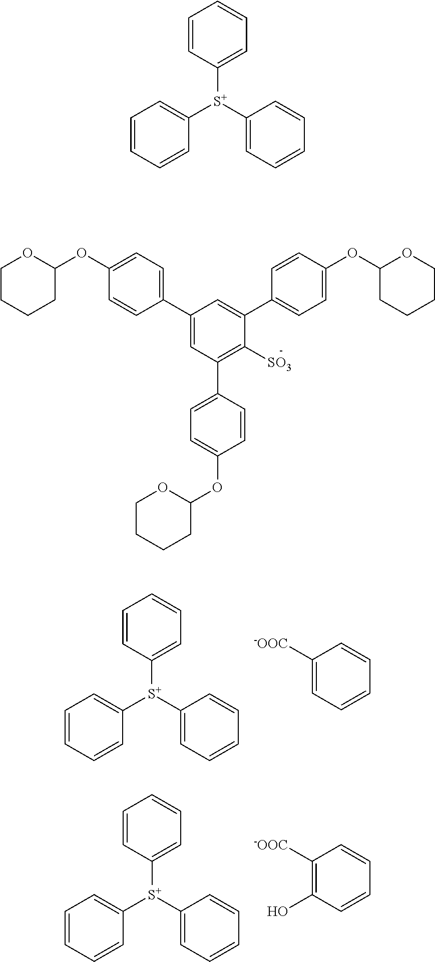

### [Method for Producing Onium Salt Compound (A)]

A method for producing an onium salt compound (A) for an actinic ray-sensitive or radiation-sensitive resin composition of the embodiment of the present invention includes:
a step (step (X)) of mixing an onium salt compound (1) represented by Formula (1A) or Formula (1B) with a salt compound (2) represented by Formula (2) in a non-aqueous solvent (S) to obtain an onium salt compound (3) represented by Formula (3A) or Formula (3B); and
a step (step (Y)) of obtaining an onium salt compound (A) represented by Formula (4A) or Formula (4B) from the onium salt compound (3).

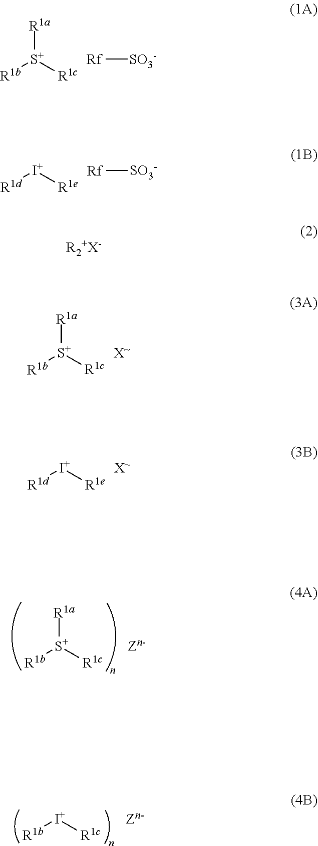

In Formulae (1A), (3A), and (4A), R^{1a}, R^{1b}, and R^{1c} each independently represent an alkyl group, a cycloalkyl group, an aryl group, or *—W₁—C(═O)Ar¹. Two of R^{1a}, R^{1b}, and R^{1c} may be bonded to each other to form a ring. *represents a linkage site to S⁺, W₁ represents a single bond or an alkylene group, and Ar represents an aryl group.

In Formulae (1B), (3B), and (4B), R^{1d} and R^{1e} each independently represent an alkyl group, a cycloalkyl group, or an aryl group.

In Formulae (1A) and (1B), Rf represents an alkyl group or an aryl group which includes one or more fluorine atoms.

In Formula (2), R₂⁺ represents an organic cation having no polymer structure. X⁻ represents Cl⁻ or Br⁻.

In Formulae (3A) and (3B), X⁻ represents Cl⁻ or Br⁻.

In Formulae (4A) and (4B), Zⁿ⁻ represents an n-valent organic anion. n represents an integer of 1 or more.

### <Step (X)>

The step (X) is a step (step (X)) of mixing an onium salt compound (1) represented by Formula (1A) or Formula (1B) with a salt compound (2) represented by Formula (2) in a non-aqueous solvent (S) to obtain an onium salt compound (3), which is a synthetic intermediate, represented by Formula (3A) or Formula (3B).

### (Onium Salt Compound (1))

The onium salt compound (1) used as a raw material substance is an onium salt compound represented by Formula (1A) or Formula (1B).

R^{1a}, R^{1b}, and R^{1c} in Formula (1A) have the same meanings as R^{1a}, R^{1b}, and R^{1c} in Formula (4A) described above, and preferred examples thereof are also the same.

R^{1d} and R^{1e} in Formula (1B) have the same meanings as R^{1d} and R^{1e} in Formula (4B) described above, and preferred examples thereof are also the same.

In Formulae (1A) and (1B), Rf represents an alkyl group or an aryl group which includes one or more fluorine atoms.

As the alkyl group having one or more fluorine atoms represented by Rf, a linear or branched alkyl group having one or more fluorine atoms, each having 1 to 4 carbon atoms, is preferable, a linear or branched perfluoroalkyl group having one or more fluorine atoms, each having 1 to 4 carbon atoms, is more preferable, and a trifluoromethyl group is still more preferable.

As the aryl group having one or more fluorine atoms, represented by Rf, a phenyl group having one or more fluorine atoms or a naphthyl group is preferable.

Rf preferably represents a linear or branched perfluoroalkyl group having 1 to 4 carbon atoms, and Rf—SO₃— preferably represents a triflate anion.

### (Salt Compound (2))

The salt compound (2) is a salt compound represented by Formula (2).

In Formula (2), R₂⁺ represents an organic cation having no polymer structure. The fact that a polymer structure is not provided means that the partial structure containing a cation does not form a part of the main chain of the polymer, and the partial structure containing a cation is not carried on the polymer side chain, for example, the polymer structure is not a structure like an anion exchange resin.

Examples of the organic cation represented by R₂⁺ include an ammonium cation, a phosphonium cation, a pyridinium cation, and an imidazolium cation, and an ammonium cation is preferable and an organic cation represented by Formula (2A) is more preferable.

Q⁺(R^{2e})ₘH₄₋ₘ (2A)

In Formula (2A), Q represents an N atom or a P atom, m represents an integer of 1 to 4, R^{2e} represents an alkyl group, a cycloalkyl group, or an aryl group, and a plurality of R^{2e}'s may be the same as or different from each other. R^{2e}'s adjacent to each other may form a ring. As the alkyl group represented by R^{2e}, a linear or branched alkyl group having 1 to 20 carbon atoms is preferable, and a linear alkyl group having 1 to 20 carbon atoms is more preferable.

Examples of the cycloalkyl group represented by R^{2e} include a cycloalkyl group having 3 to 15 carbon atoms.

Examples of the aryl group represented by R^{2e} include an aryl group having 6 to 14 carbon atoms, and a phenyl group is preferable.

The alkyl group, the cycloalkyl group, or the aryl group, which is represented by R^{2e}, may have a substituent, and examples of the substituent include an alkyl group (for example, having 1 to 15 carbon atoms), a cycloalkyl group (for example, having 3 to 15 carbon atoms), an aryl group (for example, having 6 to 14 carbon atoms), an alkoxy group (for example, having 1 to 15 carbon atoms), and a cycloalkylalkoxy group (for example, having 1 to 15 carbon atoms).

R^{2e}'s adjacent to each other may form a ring.

As the organic cation represented by R₂⁺, an organic cation represented by Formula (2B) is more preferable.

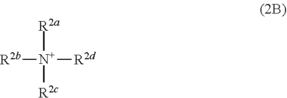

In Formula (2B), R^{2a} to R^{2d} each independently represent an alkyl group, a cycloalkyl group, or an aryl group.

As the alkyl group represented by R^{2a} to R^{2d}, a linear or branched alkyl group having 1 to 20 carbon atoms is preferable, and a linear alkyl group having 1 to 20 carbon atoms is more preferable.

Examples of the cycloalkyl group represented by R^{2a} to R^{2d} include a cycloalkyl group having 3 to 15 carbon atoms.

Examples of the aryl group represented by R^{2a} to R^{2d} include an aryl group having 6 to 14 carbon atoms, and a phenyl group is preferable.

The alkyl group, the cycloalkyl group, or the aryl group represented by R^{2a} to R^{2d} may have a substituent, and examples of the substituent include an alkyl group (for example, having 1 to 15 carbon atoms), a cycloalkyl group (for example, having 3 to 15 carbon atoms), an aryl group (for example, having 6 to 14 carbon atoms), an alkoxy group (for example, having 1 to 15 carbon atoms), and a cycloalkylalkoxy group (for example, having 1 to 15 carbon atoms).

### (Onium Salt Compound (3))

The onium salt compound (3), which is a synthetic intermediate obtained by the step (X), is an onium salt compound represented by Formula (3A) or Formula (3B).

R^{1a}, R^{1b}, and R^{1c} in Formula (3A) have the same meanings as R^{1a}, R^{1b}, and R^{1c} in Formula (4A) described above, and preferred examples thereof are also the same.

R^{1d} and R^{1e} in Formula (3B) have the same meanings as R^{1d} and R^{1e} in Formula (4B) described above, and preferred examples thereof are also the same.

X⁻ in Formulae (3A) and (3B) has the same meaning as X⁻ in Formula (2) described above.

In the step (X), first, the onium salt compound (1) and the salt compound (2) are mixed in the non-aqueous solvent (S).

### (Non-Aqueous Solvent (S))

The non-aqueous solvent referred to here refers to all organic solvents other than water. Examples of the non-aqueous solvent (S) include a ketone-based solvent, an ester-based solvent, an alcohol-based solvent, a nitrile-based solvent, an amide-based solvent, an ether-based solvent, a halogen-based solvent, and a hydrocarbon-based solvent, and two or more kinds of solvents may be used in combination.

Examples of the ketone-based solvent include 1-octanone, 2-octanone, 1-nonanone, 2-nonanone, acetone, 2-heptanone (methyl amyl ketone), 4-heptanone, 1-hexanone, 2-hexanone, diisobutyl ketone, cyclohexanone, methylcyclohexanone, phenyl acetone, methyl ethyl ketone, methyl isobutyl ketone, acetyl acetone, acetonyl acetone, ionone, diacetonyl alcohol, acetyl carbinol, acetophenone, methyl naphthyl ketone, isophorone, and propylene carbonate. Among these, methyl ethyl ketone, methyl isobutyl ketone, and 2-heptanone are preferable.

Examples of the ester-based solvent include methyl acetate, butyl acetate, ethyl acetate, isopropyl acetate, pentyl acetate, isopentyl acetate, amyl acetate, propylene glycol monomethyl ether acetate, ethylene glycol monoethyl ether acetate, diethylene glycol monobutyl ether acetate, diethylene glycol monoethyl ether acetate, ethyl-3-ethoxypropionate, 3-methoxybutyl acetate, 3-methyl-3-methoxybutyl acetate, methyl formate, ethyl formate, butyl formate, propyl formate, ethyl lactate, butyl lactate, propyl lactate, butyl butanoate, methyl 2-hydroxyisobutyrate, isoamyl acetate, isobutyl isobutyrate, and butyl propionate. Among these, butyl acetate, ethyl acetate, isopropyl acetate, isoamyl acetate, and propylene glycol monomethyl ether acetate are preferable.

Examples of the alcohol-based solvent include alcohols such as methyl alcohol, ethyl alcohol, n-propyl alcohol, isopropyl alcohol, n-butyl alcohol, sec-butyl alcohol, tert-butyl alcohol, isobutyl alcohol, 4-methyl-2-pentanol, n-hexyl alcohol, n-heptyl alcohol, n-octyl alcohol, and n-decanol, glycol-based solvents such as ethylene glycol, diethylene glycol, and triethylene glycol; and glycol ether-based solvents such as ethylene glycol monomethyl ether, propylene glycol monomethyl ether, ethylene glycol monoethyl ether, propylene glycol monoethyl ether, diethylene glycol monomethyl ether, triethylene glycol monoethyl ether, and methoxymethyl butanol. Among these, isopropyl alcohol, n-butyl alcohol, 4-methyl-2-pentanol, or propylene glycol monomethyl ether is preferable.

Examples of the nitrile-based solvent include acetonitrile, propionitrile, and benzonitrile. Among these, acetonitrile and propionitrile are preferable.

Examples of the amide-based solvent include N-methyl-2-pyrrolidone, N,N-dimethylacetamide, N,N-dimethylformamide, hexamethylphosphoric triamide, and 1,3-dimethyl-2-imidazolidinone. Among these, N-methyl-2-pyrrolidone is preferable.

Examples of the ether-based solvent include dioxane, tetrahydrofuran, tert-butyl methyl ether, 2-methyltetrahydrofuran, cyclopentyl methyl ether, 4-methyltetrahydropyran, anisole, and the like, in addition to the above-described glycol ether-based solvent. Among these, 2-methyltetrahydrofuran, cyclopentyl methyl ether, or 4-methyltetrahydropyran is preferable.

Examples of the halogen-based solvent include methylene chloride, chloroform, 1,2-dichloroethane, tetrachloromethane, chlorobenzene, o-dichlorobenzene, and m-dichlorobenzene. Among these, chloroform and 1,2-dichloroethane are preferable.

Examples of the hydrocarbon-based solvent include aromatic hydrocarbon-based solvents such as toluene and xylene, and aliphatic hydrocarbon-based solvents such as pentane, hexane, heptane, octane, and decane. Since the solubility of the onium salt compound (1) is low in only the hydrocarbon-based solvent, it is preferable to use the hydrocarbon-based solvent in combination with the other solvent species described above, and toluene or heptane is preferable as the solvent species.

The non-aqueous solvent (S) does not necessarily have to completely dissolve the onium salt compound (1) and the salt compound (2), but it is preferable that the onium salt compound (1) is dissolved and the onium salt compound (3) is not dissolved in the solvent, since ion exchange is promoted.

Since both of the compounds are not dissolved in a low-polarity solvent and both of the compounds are dissolved in a high-polarity solvent, the non-aqueous solvent (S) is preferably a solvent having appropriate polarity. From that viewpoint, the non-aqueous solvent (S) preferably contains at least any one selected from a ketone-based solvent, an ester-based solvent, an ether-based solvent, or a halogen-based solvent, and more preferably contains at least one of an ester-based solvent or an ether-based solvent.

In a case where two or more kinds of solvents are combined, it is preferable that at least one kind of solvent is an ester-based solvent or an ether-based solvent, and it is more preferable that all solvents are composed of an ester-based solvent or an ether-based solvent.

The non-aqueous solvent (S) preferably exhibits appropriate polarity.

Examples of an indicator of appropriate polarity of the non-aqueous solvent (S) include an SP value calculated from HSPiP 5th Edition 5.1.08. The SP value (δTot) calculated by the above-described method is preferably 16.5 to 21.5, more preferably 17.0 to 20.0, and still more preferably 17.0 to 19.0. In a case where two or more kinds of solvents are combined, it is preferable that the calculated SP value of the mixed solvent is within the above-described range. The calculated SP value of the mixed solvent is obtained by obtaining an arithmetic mean of numerical values obtained by multiplying the Hansen parameters of the respective solvents constituting the mixed solvent by the volume ratio, calculating the respective Hansen parameters (δD, δP, δH) of the mixed solvent, and then calculating the SP value (δTot) from the respective Hansen parameters of the mixed solvent.

A mixing order and mixing method in a case of mixing the onium salt compound (1) and the salt compound (2) in the non-aqueous solvent (S) are not particularly limited, and for example, the onium salt compound (1) may be added to the non-aqueous solvent (S) and dissolved (may not be completely dissolved) by stirring, then the salt compound (2) may be added while stirring the solution, and after the addition of the salt compound (2) is completed, the solution may be continuously stirred for a predetermined time to mix the onium salt compound (1) and the salt compound (2). A salt exchange reaction occurs by mixing both in the non-aqueous solvent (S).

The amount ratio of the onium salt compound (1) and the chloride compound (2) used in the step (X) is determined by the stoichiometric ratio, but the molar ratio of [chloride compound (2)]/[onium salt compound (1)] is preferably 2.0 to 0.9, more preferably 1.5 to 1.0, and still more preferably 1.2 to 1.0.

In addition, the amount of the non-aqueous solvent (S) used may be, for example, 2 times to 50 times the amount of the onium salt compound (1) by mass, and is preferably 2 times to 20 times the amount of the onium salt compound (1) by mass.

The reaction temperature (mixing temperature) is not particularly limited, and is, for example, preferably set to about 0° C. to 150° C. and more preferably set to 20° C. to 100° C.

The reaction time (mixing time) is not particularly limited, and for example, may be 15 minutes to 15 hours and is preferably 30 minutes to 8 hours from the start of the reaction (that is, from the start of mixing the onium salt compound (1) and the salt compound (2)).

By going through the above-described step (X), a reaction solution including the onium salt compound (3) represented by Formula (3A) or Formula (3B) is obtained.

After the step (X) is completed, the obtained reaction solution may be subjected to isolation and purification of the onium salt compound (3) by using general separation and purification means such as washing, extraction, drying, filtration, and concentration. These separation and purification means can be used alone or in combination of two or more kinds thereof.

For example, the onium salt compound (3) can be isolated and purified by filtering the obtained reaction solution to collect crude crystals, dissolving the crude crystals in a good solvent such as a ketone-based solvent such as acetone, an alcohol solvent such as methanol, or a halogen-based solvent such as methylene chloride, and adding a poor solvent such as an ether-based solvent such as tert-butyl methyl ether, a hydrocarbon-based solvent such as n-hexane, or water, to precipitate crystals, which are then collected by filtration. In addition, the onium salt compound (3) can also be purified by dispersing the crude crystals in an ester-based solvent such as butyl acetate or an ether-based solvent such as 4-methyltetrahydropyrane, stirring the mixture at 40° C. to 80° C. for 15 minutes to 2 hours, allowing the mixture to cool, and collecting the heated, dispersed, and washed crystals by filtration.

### <Step (Y)>

The step (Y) is a step of obtaining the onium salt compound (A) represented by Formula (4A) or Formula (4B) from the onium salt compound (3).

The onium salt compound (3) as a starting material and the onium salt compound (A) as a product in the step (Y) are as described above.

In the step (Y), it is preferable that the onium salt (3) and a salt compound (5) represented by Formula (5) are reacted with each other in a solvent to obtain the onium salt compound (A).

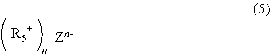

In Formula (5), R₅⁺ represents a metal ion or an organic cation. Zⁿ⁻ represents an n-valent organic anion. n represents an integer of 1 or more.

In Formula (5), R₅⁺ represents a metal ion or an organic cation.

Examples of the metal ion include a sodium ion, a potassium ion, and a silver ion.

Examples of the organic cation include an ammonium cation, a phosphonium cation, a pyridinium cation, and an imidazolium cation.

R₅⁺ is preferably an alkali metal ion.

In Formula (5), Zⁿ⁻ represents an n-valent organic anion. n represents an integer of 1 or more.

Zⁿ⁻ in Formula (5) has the same meaning as Zⁿ⁻ in Formulae (4A) and (4B) described above, and preferred examples thereof are also the same.

n in Formula (5) has the same meaning as n in Formulae (4A) and (4B) described above, and the same applies to the preferred range thereof.

For the reaction in the step (Y), for example, a general salt exchange reaction of a biphasic system using water and an organic solvent can be adopted. The onium salt compound (3) and the salt compound (5) are mixed in a mixed solvent of water and an organic solvent, whereby a salt exchange reaction occurs.

As the organic solvent, a solvent that separates from water to remove the by-produced R₅⁺X⁻ in the water layer by dissolving the onium salt compound (A) is preferable, and examples thereof include methylene chloride, chloroform, and ethyl acetate.

The onium salt compound (3) and the salt compound (5) are mixed in a mixed solvent of water and an organic solvent, whereby a salt exchange reaction occurs. The mixing order and mixing method in a case of mixing the onium salt compound (3) and the salt compound (5) in the mixed solvent are not particularly limited, and for example, the salt compound (5) may be added to the mixed solvent and mixed by stirring, adding the onium salt compound (3) while stirring the solution, and after the addition of the salt compound (3) is completed, the solution may be continuously stirred for a predetermined time to mix the onium salt compound (3) and the salt compound (5).

The amount ratio of the onium salt compound (3) and the chlorine compound (5) used in the step (Y) is determined by the stoichiometric ratio, but the molar ratio of [onium salt compound (3)/n]/[chlorine compound (5)] is preferably 1.2 to 0.8 and more preferably 1.1 to 0.9.

In addition, the amount of the solvent used may be, for example, 2 times to 50 times the amount of the onium salt compound (3) by mass, and is preferably 2 times to 20 times the amount of the onium salt compound (3) by mass.

The amount ratio of water to the organic solvent in the solvent may be, for example, 1.5 to 0.5 in terms of the volume ratio of organic solvent/water.

The reaction temperature (mixing temperature) is not particularly limited, and is, for example, preferably set to about 0° C. to 80° C. and more preferably set to 10° C. to 50° C.

The reaction time (mixing time) is not particularly limited, and for example, may be 10 minutes to 4 hours and is preferably 15 minutes to 2 hours from the start of the reaction (that is, from the start of mixing the onium salt compound (3) and the salt compound (5)).

By going through the above-described step (Y), a reaction solution including the onium salt compound (A) represented by Formula (4A) or Formula (4B) is obtained.

After the step (Y) is completed, the onium salt compound (A) may be subjected to isolation and purification by using general separation and purification means such as washing, extraction, drying, filtration, concentration, and recrystallization. These separation and purification means can be used alone or in combination of two or more kinds thereof.

For example, the water phase of the obtained reaction solution is removed, the organic phase is washed with an aqueous acid solution or water, and then concentrated under reduced pressure to obtain a crude product. A poor solvent such as diisopropyl ether, tert-butyl methyl ether, or cyclopentyl methyl ether is added to the crude product, and the mixture is stirred, and the precipitated solid is collected by filtration, whereby the onium salt compound (A) can be isolated and purified. In addition, purification means such as crystallization, recrystallization, and silica gel column chromatography can also be applied.

By using the method for producing an onium salt compound according to the embodiment of the present invention, the residual rate of impurities such as the onium salt compound (1) which is a raw material compound and the onium salt compound (3) which is a synthetic intermediate, which are contained in the onium salt compound (A) which is a final product, can be reduced.

Hereinafter, an evaluation method for the residual rate of these impurities will be described.

### (Evaluation Method for Residual Rate of Onium Salt Compound (1))

The residual rate of the onium salt compound (1) represented by Formula (1A) or (1B) in the onium salt compound (A) is preferably calculated by the following method.

In a case where the onium salt compound (A) has an F atom, the ratio is calculated from the ratio of the integral value of the peak A_{F} derived from the F atom of the onium salt compound (A) to the integral value of the peak 1_{F} derived from the Rf of the onium salt compound (1), by measuring the ¹⁹F NMR of a sample solution obtained by dissolving the onium salt compound (A) in a deuterated solvent. In a case where an integral value of the peak A_{F} per F atom is defined as I_{AF} and an integral value of the peak 1_{F} per F atom is defined as I_{IF}, a residual rate Y (mol %) of the onium salt compound (1) can be calculated by Expression (2-1).

$$Y = {I}_{1 F} / ( {I}_{A F} + {I}_{1 F} ) \times 100 \qquad ( 2 - 1 )$$

In a case where the onium salt compound (A) does not have a F atom, the internal standard substance P having a F atom and the onium salt compound (A) are dissolved in a deuterated solvent to prepare a sample solution. The ¹H NMR and ¹⁹F NMR of the sample solution are measured, and the integral values of the peak 1_{F} derived from the Rf of the onium salt compound (1) and the peak P_{F} derived from the F atom of the internal standard substance P in the ¹⁹F NMR are determined respectively. Similarly, in the ¹H NMR, the integral values of the peak A_{H} derived from the onium salt compound (A) and the peak P_{H} derived from the internal standard substance P are obtained, respectively. In a case where an integral value of the peak 1_{F} per F atom is defined as I_{IF}, an integral value of the peak P_{F} per F atom is defined as I_{PF}, an integral value of the peak A_{H} per H atom is defined as I_{AH}, and an integral value of the peak P_{H} per H atom is defined as I_{PH}, a residual rate Y (mol %) of the onium salt compound (1) can be calculated by Expression (2-2).

$$Y = \{ ( {I}_{PH} / {I}_{A H} ) \times ( {I}_{1 F} / {I}_{PF} ) \} / \{ ( {I}_{PH} / {I}_{A H} ) \times ( {I}_{1 F} / {I}_{PF} ) + 1 \} \times 100 \qquad ( 2 - 2 )$$

The internal standard substance P is not particularly limited as long as it has a fluorine atom, and examples thereof include 1,4-difluorobenzene, 1,4-bis(trifluoromethyl)benzene, ethyl trifluoroacetate, and 2,2,2-trifluoroethanol.

Y is preferably 2.0 mol % or less, more preferably 1.0 mol % or less, and particularly preferably 0.4 mol % or less.

### (Evaluation Method for Residual Rate of Onium Salt Compound (3))

The residual rate of the onium salt compound (3) represented by Formula (3A) or (3B) in the onium salt compound (A) is preferably calculated according to the following silver nitrate titration method.

The onium salt compound (A) is weighed to W (g) and dissolved in a solvent such as tetrahydrofuran (THF) to prepare a sample solution. Using a silver nitrate aqueous solution of C(N), the amount of the titrant is measured for an empty solution of only a solvent and the above-described sample solution with an automatic titrator (AT-510, manufactured by KYOTO ELECTRONICS MANUFACTURING Co., Ltd.). From the obtained results of the amount of the titration, the amount Q (ppm) of the halogen is calculated using the following expression.

$$Q ( ppm ) = ( V 1 - V 2 ) \times C \times f \times M Q \times 1 \; 000 / W$$

In the expression, V1 represents the amount of the titrant of a sample solution (ml), V2 represents the amount of the titrant of an empty solution (ml), f represents a power value of a titrant, MQ represents a molar mass (g/mol) of a halogen atom to be obtained, and W represents a weighed value of the onium salt compound (A).

Assuming that all the Qs obtained above are due to the residual of the onium salt compound (3), a residual rate X (mol %) of the onium salt compound (3) with respect to 1 mol of the onium salt compound (A) is calculated using the following Expression (1).

$$X = Q \times MA / MQ / 10000 \qquad ( 1 )$$

In the formula, MA (g/mol) represents the molecular weight of the onium salt compound (A).

The concentration of the silver nitrate aqueous solution used is preferably 0.01 N or less. The solvent for dissolving the onium salt compound (A) is not particularly limited as long as it is a polar solvent that is water-soluble and does not react with silver nitrate, but is preferably an ether-based solvent such as THF or an ester-based solvent such as γ-butyrolactone, and more preferably a mixed solvent of the above-described solvent and water.

X is preferably 2.0 mol % or less, more preferably 1.0 mol % or less, and particularly preferably 0.4 mol % or less.

The onium salt compound (A) produced by the above-described method for producing is preferably used as a photoacid generator described later.

In addition, a preferred aspect thereof includes using the onium salt compound (A) as an acid diffusion control agent described later.

In a case where the onium salt compound (A) is used as the acid diffusion control agent, it is preferable to use a photoacid generator such that an acid generated from the photoacid generator is a relatively strong acid with respect to an acid generated from the onium salt compound (A) and necessary for the reaction of the resin in the exposed portion.

The content of the onium salt compound (A) is preferably 3% by mass or more, more preferably 5% by mass or more, and still more preferably 10% by mass or more with respect to the total solid content of the composition.

The upper limit value of the content of the onium salt compound (A) is not particularly limited, but is usually 60% by mass or less, preferably 50% by mass or less, and more preferably 45% by mass or less with respect to the total solid content of the composition.

The onium salt compound (A) may be used alone or in combination of two or more kinds thereof.

### [Resin (B)]

The resin (B) contained in the composition of the embodiment of the present invention is a resin whose solubility in a developer changes due to action of acid.

The resin (B) usually includes a repeating unit having a group having a polarity that increases through decomposition by the action of acid (hereinafter, also referred to as an “acid-decomposable group”), and preferably includes a repeating unit having an acid-decomposable group.

In a case where the resin (B) has an acid-decomposable group, in the pattern forming method in the present specification, typically, in a case where an alkali developer is adopted as a developer, a positive tone pattern is suitably formed, and in a case where an organic developer is adopted as a developer, a negative tone pattern is suitably formed.

### (Repeating Unit Having Acid-Decomposable Group)

The acid-decomposable group is a group which is decomposed by action of acid to form a polar group. The acid-decomposable group preferably has a structure in which a polar group is protected by a group that leaves by action of acid (leaving group). That is, the resin (B) has a repeating unit having a group which is decomposed by action of acid to generate a polar group. The resin having this repeating unit has an increased polarity by action of acid, an increased solubility in an alkali developer, and a decreased solubility in an organic solvent.

Specific examples of the repeating unit having an acid-decomposable group include repeating units described in paragraphs [0104] to [0149] of WO2021/251086A.

A content of the repeating unit having an acid-decomposable group is preferably 15% by mole or more, more preferably 20% by mole or more, and still more preferably 30% by mole or more with respect to all repeating units in the resin (B). In addition, the upper limit value thereof is preferably 90% by mole or less, more preferably 80% by mole or less, still more preferably 70% by mole or less, and particularly preferably 60% by mole or less with respect to all repeating units in the resin (B).

The resin (B) may include at least one repeating unit selected from the group consisting of the following group A and/or at least one repeating unit selected from the group consisting of the following group B.

Group A: group consisting of the following repeating units (20) to (25)
(20) repeating unit having an acid group, which will be described later
(21) repeating unit having neither an acid-decomposable group nor an acid group and having a fluorine atom, a bromine atom, or an iodine atom, which will be described later
(22) repeating unit having a lactone group, a sultone group, or a carbonate group, which will be described later
(23) repeating unit having a photoacid generating group, which will be described later
(24) repeating unit represented by Formula (V-1) or Formula (V-2), which will be described later
(25) repeating unit for reducing mobility of main chain

Group B: group consisting of the following repeating units (30) to (32)
(30) repeating unit having at least one group selected from a hydroxyl group, a cyano group, or an alkali-soluble group, which will be described later
(31) repeating unit having an alicyclic hydrocarbon structure and not exhibiting acid decomposability, which will be described later
(32) repeating unit represented by Formula (III) having neither a hydroxyl group nor a cyano group, which will be described later

As a preferred aspect of the resin (B), an aspect in which the resin (B) includes at least one of a repeating unit having a phenolic hydroxyl group or a repeating unit having a lactone group is exemplified. In this manner, the adhesiveness of the resist film formed of the composition of the embodiment of the present invention to the substrate is improved.

The resin (B) preferably has an acid group, and preferably includes a repeating unit having an acid group. In a case where the resin (B) has an acid group, the interaction between the resin (B) and the acid generated from the photoacid generator is more excellent. As a result, diffusion of the acid is further suppressed, and a cross-sectional shape of the formed pattern can be more rectangular.

The resin (B) may have at least one repeating unit selected from the group consisting of the group A. In a case where the composition according to the embodiment of the present invention is used as an actinic ray-sensitive or radiation-sensitive resin composition with EUV exposure, it is preferable that the resin (B) has at least one repeating unit selected from the group consisting of the above-described group A.

The resin (B) may contain at least one of a fluorine atom or an iodine atom. In a case where the composition according to the embodiment of the present invention is used as an actinic ray-sensitive or radiation-sensitive resin composition with EUV exposure, it is preferable that the resin (B) includes at least one of a fluorine atom or an iodine atom. In a case where the resin (B) includes both a fluorine atom and an iodine atom, the resin (B) may have one repeating unit including both a fluorine atom and an iodine atom, and the resin (B) may include two kinds of repeating units, that is, a repeating unit having a fluorine atom and a repeating unit having an iodine atom.

The resin (B) may have at least one repeating unit selected from the group consisting of the group B. In a case where the composition according to the embodiment of the present invention is used as an actinic ray-sensitive or radiation-sensitive resin composition with ArF, it is preferable that the resin (B) has at least one repeating unit selected from the group consisting of the above-described group B.

In a case where the composition according to the embodiment of the present invention is used as an actinic ray-sensitive or radiation-sensitive resin composition with ArF, it is preferable that the resin (B) does not include a fluorine atom and a silicon atom.

### (Repeating Unit Having Acid Group)

The resin (B) may have a repeating unit having an acid group.

As the acid group, an acid group having a pKa of 13 or less is preferable. An acid dissociation constant of the above-described acid group is preferably 13 or less, more preferably 3 to 13, and still more preferably 5 to 10.

In a case where the resin (B) has an acid group having a pKa of 13 or less, a content of the acid group in the resin (B) is not particularly limited, but is usually 0.2 to 6.0 mmol/g. Among these, 0.8 to 6.0 mmol/g is preferable, 1.2 to 5.0 mmol/g is more preferable, and 1.6 to 4.0 mmol/g is still more preferable. In a case where the content of the acid group is within the above-described range, the development proceeds satisfactorily, the formed pattern shape is excellent, and the resolution is also excellent.

As the acid group, for example, a carboxyl group, a phenolic hydroxyl group, a fluoroalcohol group (preferably, a hexafluoroisopropanol group), a sulfonic acid group, a sulfonamide group, or an isopropanol group is preferable.

In the above-described hexafluoroisopropanol group, one or more (preferably one or two) fluorine atoms may be substituted with a group (an alkoxycarbonyl group and the like) other than a fluorine atom.

—C(CF₃)(OH)—CF₂— formed as above is also preferable as the acid group. In addition, one or more fluorine atoms may be substituted with a group other than a fluorine atom to form a ring including —C(CF₃)(OH)—CF₂—.

The repeating unit having an acid group is preferably a repeating unit different from a repeating unit having the structure in which a polar group is protected by the group that is eliminated by the action of an acid as described above, and a repeating unit having a lactone group, a sultone group, or a carbonate group which will be described later.

The repeating unit having an acid group may have a fluorine atom or an iodine atom.

In a case where the resin (B) includes a repeating unit having an acid group, a content of the repeating unit having an acid group is preferably 5 mol % or more and more preferably 10 mol % or more with respect to all the repeating units in the resin (B). In addition, the upper limit value thereof is preferably 70% by mole or less, more preferably 65% by mole or less, and still more preferably 60% by mole or less with respect to all repeating units in the resin (B).

### (Repeating Unit Having Neither Acid-decomposable Group Nor Acid Group and Having Fluorine Atom, Bromine Atom, or Iodine Atom)

The resin (B) may have a repeating unit (hereinafter, also referred to as a unit X) having a fluorine atom, a bromine atom, or an iodine atom, which has neither an acid-decomposable group nor an acid group, in addition to the above-described acid-decomposable repeating unit and repeating unit having an acid group. It is preferable that the <repeating unit having neither an acid-decomposable group nor an acid group and having a fluorine atom, a bromine atom, or an iodine atom> herein is different from other types of the repeating units belonging to the group A, such as <repeating unit having lactone group, sultone group, or carbonate group> and <repeating unit having photoacid generating group> described later.

### (Repeating Unit Having Lactone Group, Sultone Group, or Carbonate Group)

The resin (B) may have a repeating unit having at least one selected from the group consisting of a lactone group, a sultone group, and a carbonate group (hereinafter, also referred to as “unit Y”).

It is also preferable that the unit Y has no hydroxyl group and acid group such as a hexafluoroisopropanol group.

The lactone group or the sultone group may have a lactone structure or a sultone structure. The lactone structure or the sultone structure is preferably a 5- to 7-membered ring lactone structure or a 5- to 7-membered ring sultone structure. Among these, the structure is more preferably a 5- to 7-membered ring lactone structure with which another ring structure is fused so as to form a bicyclo structure or a spiro structure or a 5- to 7-membered ring sultone structure with which another ring structure is fused so as to form a bicyclo structure or a spiro structure.

Specific examples of the repeating unit having a preferred lactone group, sultone group, or carbonate group include those described in paragraphs [0193] to [0207] of WO2021/251086A.

In a case where the resin (B) includes the unit Y, a content of the unit Y is preferably 1 mol % or more, and more preferably 5 mol % or more with respect to all repeating units in the resin (B). In addition, the upper limit value thereof is preferably 85% by mole or less, more preferably 80% by mole or less, still more preferably 70% by mole or less, and particularly preferably 60% by mole or less with respect to all repeating units in the resin (A).

### (Repeating Unit Having Photoacid Generating Group)

The resin (B) may have a repeating unit having a group (hereinafter, also referred to as a “photoacid generating group”) which generates an acid upon irradiation with actinic rays or radiation (preferably, electron beams or extreme ultraviolet rays) as a repeating unit in addition to the above-mentioned repeating units.

Examples of a preferred aspect of the resin (B) include an aspect in which the resin (B) includes a repeating unit having a group which decomposes upon irradiation with electron beams or extreme ultraviolet rays to generate an acid.

Examples of the repeating unit having a photoacid generating group include the repeating units described in paragraphs [0094] to [0105] of JP2014-041327A and the repeating units described in paragraph [0094] of WO2018/193954A.

### (Repeating Unit Represented by Formula (V-1) or Formula (V-2))

The resin (B) may have a repeating unit represented by Formula (V-1) or Formula (V-2).

The repeating unit represented by Formula (V-1) and Formula (V-2) is preferably a repeating unit different from the above-described repeating units.

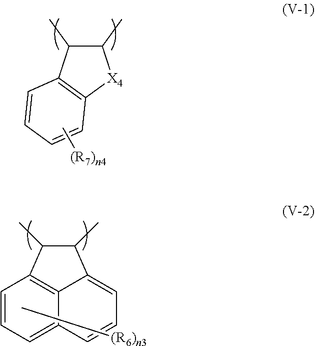

In the formulae,

R₆ and R₇ each independently represent a hydrogen atom, a hydroxyl group, an alkyl group, an alkoxy group, an acyloxy group, a cyano group, a nitro group, an amino group, a halogen atom, an ester group (—OCOR or —COOR; R is an alkyl group or fluorinated alkyl group having 1 to 6 carbon atoms), or a carboxyl group. As the alkyl group, a linear, branched, or cyclic alkyl group having 1 to 10 carbon atoms is preferable.

n₃ represents an integer of 0 to 6.

n₄ represents an integer of 0 to 4.

X₄ is a methylene group, an oxygen atom, or a sulfur atom.

The repeating unit represented by Formula (V-1) or (V-2) is exemplified below.

Examples of the repeating unit represented by Formula (V-1) or (V-2) include repeating units described in paragraph [0100] of WO2018/193954A.

### (Repeating Unit for Reducing Mobility of Main Chain)

From the viewpoint that excessive diffusion of a generated acid or pattern collapse during development can be suppressed, the resin (B) preferably has a high glass transition temperature (Tg). The Tg is preferably higher than 90° C., more preferably higher than 100° C., still more preferably higher than 110° C., and particularly preferably higher than 125° C. From the viewpoint that a dissolution rate in the developer is excellent, the Tg is preferably 400° C. or lower and more preferably 350° C. or lower.

In order to raise the Tg of the resin (B) (preferably to raise the Tg to higher than 90° C.), it is preferable to reduce the mobility of the main chain of the resin (B). Examples of a method for lowering the mobility of the main chain of the resin (B) include the following (a) to (e) methods.
(a) introduction of a bulky substituent into the main chain
(b) introduction of a plurality of substituents into the main chain
(c) introduction of a substituent causing an interaction between the resins (B) into the vicinity of the main chain
(d) formation of the main chain in a cyclic structure
(e) linking of a cyclic structure to the main chain

The resin (B) preferably has a repeating unit in which the homopolymer exhibits a Tg of 130° C. or higher.

Examples of specific accomplishing means for (a) to (e) above include a method of introducing a repeating unit described in paragraphs [0107] to [0133] of WO2018/193954A into the resin (B).

(Repeating Unit Having at least One Group Selected from Hydroxyl Group, Cyano Group, or Alkali-Soluble Group)

The resin (B) may have a repeating unit having at least one group selected from a hydroxyl group, a cyano group, or an alkali-soluble group.

The resin (B) may have a repeating unit having a hydroxyl group or a cyano group. As a result, adhesiveness to the substrate and affinity for a developer are improved.

The repeating unit having a hydroxyl group or a cyano group is preferably a repeating unit having an alicyclic hydrocarbon structure substituted with a hydroxyl group or a cyano group.

It is preferable that the repeating unit having a hydroxyl group or a cyano group does not have the acid-decomposable group. Examples of the repeating unit having a hydroxyl group or a cyano group include repeating units described in paragraphs [0081] to [0084] of JP2014-098921A.

The resin (B) may have a repeating unit having an alkali-soluble group.

Examples of the alkali-soluble group include a carboxyl group, a sulfonamide group, a sulfonylimide group, a bissulfonylimide group, and an aliphatic alcohol group (for example, a hexafluoroisopropanol group) in which the α-position is substituted with an electron withdrawing group, and a carboxyl group is preferable. In a case where the resin (B) includes the repeating unit having an alkali-soluble group, resolution for use in contact holes is increased. Examples of the repeating unit having an alkali-soluble group include repeating units described in paragraphs [0085] and [0086] of JP2014-098921A.

### (Repeating Unit Having Alicyclic Hydrocarbon Structure and Not Exhibiting Acid Decomposability)

The resin (B) may have a repeating unit having an alicyclic hydrocarbon structure and not exhibiting acid decomposability. As a result, it is possible to reduce elution of low-molecular-weight components from the resist film into the immersion liquid during liquid immersion exposure. Examples of the repeating unit having an alicyclic hydrocarbon structure and not exhibiting acid decomposability include a repeating unit derived from 1-adamantyl (meth)acrylate, diamantyl (meth)acrylate, tricyclodecanyl (meth)acrylate, or cyclohexyl (meth)acrylate.

### (Repeating Unit Represented by Formula (III) Having Neither Hydroxyl Group Nor Cyano Group)

The resin (B) may have a repeating unit represented by Formula (III), which has neither a hydroxyl group nor a cyano group.

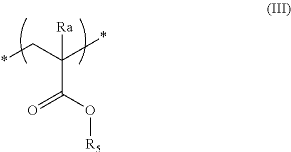

In Formula (III), R₅ represents a hydrocarbon group having at least one cyclic structure and having neither a hydroxyl group nor a cyano group.

Ra represents a hydrogen atom, an alkyl group, or a —CH₂—O—Ra₂ group. In the formula, Ra₂ represents a hydrogen atom, an alkyl group, or an acyl group.

Examples of the repeating unit represented by Formula (III), which has neither a hydroxyl group nor a cyano group, include repeating units described in paragraphs [0087] to [0094] of JP2014-098921A.

### (Other Repeating Units)

Furthermore, the resin (B) may have another repeating unit in addition to the above-described repeating units.

For example, the resin (A) may have a repeating unit selected from the group consisting of a repeating unit having an oxathiane ring group, a repeating unit having an oxazolone ring group, a repeating unit having a dioxane ring group, and a repeating unit having a hydantoin ring group.

Examples of the other repeating units include repeating units described in paragraphs [0242] to [0245] of WO2021/251086A.

The resin (B) can be synthesized in accordance with an ordinary method (for example, a radical polymerization).

A weight-average molecular weight (Mw) of the resin (B) as a value expressed in terms of polystyrene by a GPC method is preferably 30,000 or less, more preferably 1,000 to 30,000, still more preferably 3,000 to 30,000, and particularly preferably 5,000 to 15,000.

The dispersity (molecular weight distribution, Pd, Mw/Mn) of the resin (B) is preferably 1 to 5, more preferably 1 to 3, still more preferably 1.2 to 3.0, and particularly preferably 1.2 to 2.0. As the dispersity is smaller, resolution and resist shape are more excellent, and a side wall of a resist pattern is smoother and roughness is also more excellent.

The resin (B) contained in the composition according to the embodiment of the present invention may be one kind or two or more kinds.

In the composition according to the embodiment of the present invention, the content of the resin (B) is preferably 40.0% to 99.9% by mass, and more preferably 60.0% to 90.0% by mass, with respect to the total solid content of the composition according to the embodiment of the present invention.

### [Salt of Acid]

The composition according to the embodiment of the present invention preferably contains a salt of an acid, more preferably contains a salt of an acid having a pKa of −2.0 or more, and still more preferably contains a salt of an acid having a pKa of −2.0 or more and 1.0 or less.

The salt of an acid is preferably a compound (a photoacid generator) which generates an acid in a case of being irradiated with actinic ray or radiation.

### [Photoacid Generator (C)]

The photoacid generator may be in a form of a low-molecular-weight compound or a form incorporated into a part of a polymer. In addition, a combination of the form of a low-molecular-weight compound and the form incorporated into a part of a polymer may also be used.

In a case where the photoacid generator is in the form of a low-molecular-weight compound, a molecular weight of the photoacid generator is preferably 3,000 or less, more preferably 2,000 or less, and still more preferably 1,000 or less. The lower limit thereof is not particularly limited, and is preferably 100 or more.

In a case where the photoacid generator is in the form incorporated into a part of a polymer, it may be incorporated into the part of the resin (B) or into a resin which is different from the resin (B).

The photoacid generator is preferably in the form of a low-molecular-weight compound.

The photoacid generator is preferably a compound which generates an acid having a pKa of −2.0 or more in a case of being irradiated with actinic ray or radiation, and more preferably a compound which generates an acid having a pKa of −2.0 or more and 1.0 or less.

The photoacid generator may or may not be the above-described onium salt compound (A).

Examples of the photoacid generator include a compound (onium salt) represented by “M⁺ X⁻”, and a compound that generates an organic acid by exposure is preferable.

Examples of the above-described organic acid include sulfonic acids (an aliphatic sulfonic acid, an aromatic sulfonic acid, a camphor sulfonic acid, and the like), carboxylic acids (an aliphatic carboxylic acid, an aromatic carboxylic acid, an aralkylcarboxylic acid, and the like), a carbonylsulfonylimide acid, a bis(alkylsulfonyl)imide acid, and a tris(alkylsulfonyl)methide acid.

In the compound represented by “M⁺ X⁻”, M⁺ represents an organic cation.

The organic cation is not particularly limited. The valence of the organic cation may be monovalent or divalent or higher valent.

Among these, as the above-described organic cation, a cation represented by Formula (ZaI) (hereinafter, also referred to as “cation (ZaI)”) or a cation represented by Formula (ZaII) (hereinafter, also referred to as “cation (ZaII)”) is preferable.

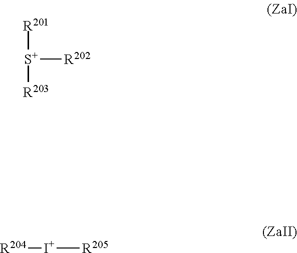

In Formula (ZaI), R²⁰¹, R²⁰², and R²⁰³ each independently represent an organic group.

The number of carbon atoms in the organic group of R²⁰¹, R²⁰², and R²⁰³ is preferably 1 to 30 and more preferably 1 to 20. Two of R²⁰¹ to R²⁰³ may be bonded to each other to form a ring structure, and the ring structure may include an oxygen atom, a sulfur atom, an ester group, an amide group, or a carbonyl group in the ring. Examples of the group formed by the bonding of two of R²⁰¹ to R²⁰³ include an alkylene group (for example, a butylene group and a pentylene group) and —CH₂—CH₂—O—CH₂—CH₂—.

Specific examples of the organic cation represented by M⁺ include the cations described in paragraphs [0064] to [0086] of WO2021/251086A.

In the compound represented by “M⁺ X⁻”, X⁻ represents an organic anion.

The organic anion is not particularly limited, and examples thereof include a monovalent or di- or higher valent organic anion.

The organic anion is preferably an anion with significantly lower ability to undergo nucleophilic reaction, and more preferably a non-nucleophilic anion.

Examples of the non-nucleophilic anion include a sulfonate anion (an aliphatic sulfonate anion, an aromatic sulfonate anion, a camphor sulfonate anion, and the like), a carboxylate anion (an aliphatic carboxylate anion, an aromatic carboxylate anion, an aralkyl carboxylate anion, and the like), a sulfonylimide anion, a bis(alkylsulfonyl)imide anion, and a tris(alkylsulfonyl)methide anion.

Specific examples of the organic anion represented by X⁻ include monovalent anions among the anions exemplified as the organic anion represented by Zⁿ⁻ in Formula (4A) or (4B) described above.

It is also preferable that the photoacid generator is at least one selected from the group consisting of the above-described compounds (I) and (II).

Specific examples of the photoacid generator include the photoacid generators described in paragraphs [0168] to [0171] of WO2018/193901A.

The content of the photoacid generator is not particularly limited, but from the viewpoint of further rectangularizing the cross-sectional shape of a pattern thus formed, the content thereof is preferably 0.5% by mass or more, and more preferably 1.0% by mass or more with respect to the total solid content of the composition according to the embodiment of the present invention. The above-described content is preferably 50.0% by mass or less, more preferably 30.0% by mass or less, and still more preferably 25.0% by mass or less with respect to the total solid content of the composition according to the embodiment of the present invention.

The photoacid generator may be used alone or in combination of two or more kinds thereof.

### [Acid Diffusion Control Agent (D)]

The composition according to the embodiment of the present invention may contain an acid diffusion control agent.

The acid diffusion control agent may or may not be the above-described onium salt compound (A).

The acid diffusion control agent acts as a quencher that suppresses a reaction of an acid-decomposable resin in the unexposed portion by excessive generated acids by trapping the acids generated from a photoacid generator and the like upon exposure.

The type of the acid diffusion control agent is not particularly limited, and examples thereof include a basic compound (DA), a low-molecular-weight compound (DB) having a nitrogen atom and a group which is eliminated by action of acid, and a compound (DC) in which ability to control acid diffusion decreases or disappears in a case of being irradiated with actinic ray or radiation.

Examples of the compound (DC) include an onium salt compound (DD) which is a relatively weak acid with respect to the photoacid generator, and a basic compound (DE) in which basicity decreases or disappears in a case of being irradiated with actinic ray or radiation.

Specific examples of the basic compound (DA) include compounds described in paragraphs [0132] to [0136] of WO2020/066824A, specific examples of the basic compound (DE) in which basicity decreases or disappears in a case of being irradiated with actinic ray or radiation include compounds described in paragraphs [0137] to [0155] of WO2020/066824A and compounds described in paragraph [0164] of WO2020/066824A, and specific examples of the low-molecular-weight compound (DB) having a nitrogen atom and a group which is eliminated by action of acid include compounds described in paragraphs [0156] to [0163] of WO2020/066824A.

Specific examples of the onium salt compound (DD) which is a relatively weak acid with respect to the photoacid generator include compounds described in paragraphs [0305] to [0314] of WO2020/158337A.

In addition to above, for example, as the acid diffusion control agent, known compounds described in paragraphs [0627] to [0664] of US2016/0070167A1, paragraphs [0095] to [0187] of US2015/0004544A1, paragraphs [0403] to [0423] of US2016/0237190A1, and paragraphs [0259] to [0328] of US2016/0274458A1 can be suitably used.

In a case where the composition according to the embodiment of the present invention contains the acid diffusion control agent, a content of the acid diffusion control agent (in a case of a plurality of types, the total thereof) is preferably 0.1% to 15.0% by mass, and more preferably 1.0% to 15.0% by mass with respect to the total solid content of the composition according to the embodiment of the present invention.

In the composition according to the embodiment of the present invention, the acid diffusion control agent may be used alone or in combination of two or more kinds thereof.

### [Hydrophobic Resin (E)]

The composition according to the embodiment of the present invention may further contain a hydrophobic resin (also referred to as a “hydrophobic resin (E)”) different from the resin (B).

The hydrophobic resin (E) is preferably designed to be unevenly distributed on the surface of the actinic ray-sensitive or radiation-sensitive film, but unlike the surfactant, it does not necessarily have a hydrophilic group in the molecule, not does it necessarily contribute to the uniform mixing of the polar substance and the non-polar substance.

Examples of the effects of the addition of the hydrophobic resin (E) include control of static and dynamic contact angles of the actinic ray-sensitive or radiation-sensitive film surface with respect to water and suppression of outgas.

The hydrophobic resin (E) preferably has any one or more of a fluorine atom, a silicon atom, and a CH₃ partial structure which is contained in a side chain moiety of a resin, from the viewpoint of uneven distribution on the film surface layer, and more preferably has two or more kinds thereof. The above-described hydrophobic resin preferably has a hydrocarbon group having 5 or more carbon atoms. These groups may be included in the main chain of the resin or may be substituted in the side chain of the resin.

Examples of the hydrophobic resin (E) include the compounds described in paragraphs [0275] to [0279] of WO2020/004306A.

In a case where the composition according to the embodiment of the present invention contains the hydrophobic resin (E), the content of the hydrophobic resin (E) is preferably 0.01% to 20.0% by mass, and more preferably 0.1% to 15.0% by mass with respect to the total solid content of the composition according to the embodiment of the present invention.

### [Surfactant]

The composition according to the embodiment of the present invention may contain a surfactant. In a case where the surfactant is contained, the adhesiveness is more excellent, and a pattern having fewer development defects can be formed.

The surfactant is preferably a fluorine-based and/or silicon-based surfactant.

Examples of the fluorine-based and/or silicon-based surfactant include the surfactants disclosed in paragraphs [0218] and [0219] of WO2018/193954A.

The surfactant may be used alone or in combination of two or more kinds thereof.

In a case where the composition according to the embodiment of the present invention contains a surfactant, a content of the surfactant is preferably 0.0001% to 2.0% by mass, more preferably 0.0005% to 1.0% by mass, and still more preferably 0.1% to 1.0% by mass with respect to the total solid content of the composition according to the embodiment of the present invention.

### [Solvent]

It is preferable that the composition according to the embodiment of the present invention contains a solvent.

The solvent preferably includes at least one solvent of (M1) propylene glycol monoalkyl ether carboxylate, or (M2) at least one selected from the group consisting of a propylene glycol monoalkyl ether, a lactic acid ester, an acetic acid ester, an alkoxypropionic acid ester, a chain ketone, a cyclic ketone, a lactone, and an alkylene carbonate as a solvent. The above-described solvent may further include a component other than the components (M1) and (M2).

It is preferable to combine the above-described solvent with the above-described resin from the viewpoint of improving the coating properties of the composition according to the embodiment of the present invention and reducing the number of development defects of the pattern. Since the above-described solvents have a good balance of solubility, boiling point, and viscosity of the above-described resin, unevenness in the film thickness of the resist film, generation of precipitates during spin coating, and the like can be suppressed.

Details of the component (M1) and the component (M2) are described in paragraphs [0218] to [0226] of WO2020/004306A, the contents of which are incorporated herein by reference.

In a case where the solvent further contains a component other than the components (M1) and (M2), a content of the component other than the components (M1) and (M2) is preferably 5% to 30% by mass with respect to the total amount of the solvent.

The content of the solvent in the composition of the embodiment of the present invention is preferably set so that the concentration of solid contents is 0.5% to 30% by mass, and more preferably set so that the concentration of solid contents is 1% to 20% by mass. With this content, the coating property of the composition according to the embodiment of the present invention can be further improved.

### [Other Additives]

The composition according to the embodiment of the present invention may further contain a dissolution inhibiting compound, a dye, a plasticizer, a photosensitizer, a light absorbing agent, and/or a compound promoting a solubility in a developer (for example, a phenol compound having a molecular weight of 1,000 or less, or an alicyclic or aliphatic compound including a carboxyl group), or the like.

The above-described “dissolution inhibiting compound” is intended to be a compound having a molecular weight of 3000 or less, in which solubility in an organic developer decreases by decomposition due to action of acid.

The composition according to the embodiment of the present invention is suitably used as a photosensitive composition for EB or EUV exposure.

### [Method for Producing Actinic Ray-Sensitive or Radiation-Sensitive Resin Composition]

The method for producing the composition according to the embodiment of the present invention includes:
a step (X) of mixing an onium salt compound (1) represented by Formula (1A) or Formula (1B) with a salt compound (2) represented by Formula (2) in a non-aqueous solvent (S) to obtain an onium salt compound (3) represented by Formula (3A) or Formula (3B);
a step (step (Y)) of obtaining an onium salt compound (A) represented by Formula (4A) or Formula (4B) from the onium salt compound (3); and
a step (step (Z)) of mixing the onium salt compound (A) with the resin (B) whose solubility in an alkali developer changes due to action of acid.

For the step (X) and the step (Y), the description of the above-mentioned method for producing the onium salt compound (A) can be cited.

The step (Z) is a step of mixing the onium salt compound (A) and the resin (B).

The mixing method is not particularly limited, and for example, the above-described respective components constituting the actinic ray-sensitive or radiation-sensitive resin composition may be mixed with each other by stirring.

The mixing temperature is not particularly limited, but for example, the mixing may be carried out at 10° C. to 80° C. from the viewpoint of suppressing the volatilization of the solvent described above.

The mixing time is not particularly limited either, but may be, for example, 30 minutes to 24 hours.

As a mixing ratio of the onium salt compound (A) and the resin (B), the amount of each of the onium salt compound (A) and the resin (B) to be used may be determined such that the content of each component in the actinic ray-sensitive or radiation-sensitive resin composition is within the above-described range.

### [Onium Salt Composition]

The present invention also relates to an onium salt composition including an onium salt compound, the onium salt composition including 0.001 mol % to 3 mol % of an organic cation represented by Formula (2A) with respect to 1 mol of the onium salt compound represented by Formula (4A) or (4B) (hereinafter, also referred to as “the onium salt composition according to the embodiment of the present invention”).

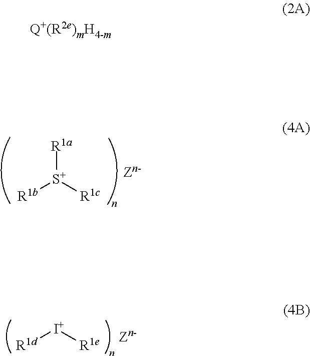

In Formula (2A), Q represents an N atom or a P atom, m represents an integer of 1 to 4, R^{2e} represents an alkyl group, a cycloalkyl group, or an aryl group, and a plurality of R^{2e}'s may be the same as or different from each other. R^{2e} 's adjacent to each other may form a ring.

In Formula (4A), R^{1a}, R^{1b}, and R^{1c} each independently represent an alkyl group, a cycloalkyl group, an aryl group, or *—W₁—C(═O)Ar¹. Two of R^{1a}, R^{1b}, and R^{1c} may be bonded to each other to form a ring. *represents a linkage site to S⁺, W₁ represents a single bond or an alkylene group, and Ar¹ represents an aryl group.

In Formula (4B), R^{1d} and R^{1e} each independently represent an alkyl group, a cycloalkyl group, or an aryl group.

In Formulae (4A) and (4B), Zⁿ⁻ represents an n-valent organic anion. n represents an integer of 1 or more.

As the alkyl group represented by R^{2e}, a linear or branched alkyl group having 1 to 20 carbon atoms is preferable, and a linear alkyl group having 1 to 20 carbon atoms is more preferable.

Examples of the cycloalkyl group represented by R^{2e} include a cycloalkyl group having 3 to 15 carbon atoms.

Examples of the aryl group represented by R^{2e} include an aryl group having 6 to 14 carbon atoms, and a phenyl group is preferable.

The alkyl group, the cycloalkyl group, or the aryl group, which is represented by R^{2e}, may have a substituent, and examples of the substituent include an alkyl group (for example, having 1 to 15 carbon atoms), a cycloalkyl group (for example, having 3 to 15 carbon atoms), an aryl group (for example, having 6 to 14 carbon atoms), an alkoxy group (for example, having 1 to 15 carbon atoms), and a cycloalkylalkoxy group (for example, having 1 to 15 carbon atoms).

R^{2e}'s adjacent to each other may form a ring.

R^{1a}, R^{1b}, and R^{1c} in Formula (4A) have the same meanings as R^{1a}, R^{1b}, and R^{1c} in Formula (4A) of the onium salt compound (A) produced by the above-described method for producing an onium salt compound according to the embodiment of the present invention, and preferred examples thereof are also the same.

R^{1d} and R^{1e} in Formula (4B) have the same meanings as R^{1d} and R^{1e} in Formula (4B) of the onium salt compound (A) produced by the method for producing an onium salt compound according to the embodiment of the present invention, and preferred examples thereof are also the same.

n and Zⁿ⁻ in Formulae (4A) and (4B) have the same meanings as n and Zⁿ⁻ in Formulae (4A) and (4B) in the onium salt compound (A) produced by the above-described method for producing an onium salt compound according to the embodiment of the present invention.

The onium salt composition according to the embodiment of the present invention is a mixture of an onium salt compound (onium salt compound (A)) represented by Formula (4A) or (4B) and a trace amount of the organic cation represented by Formula (2A).

The onium salt composition according to the embodiment of the present invention is substantially the onium salt compound containing a trace amount of the organic cation represented by Formula (2A).

The onium salt composition according to the embodiment of the present invention includes the organic cation represented by Formula (2A) (hereinafter, also referred to as an “organic cation A”) in an amount of 0.001 mol % to 3 mol % with respect to 1 mol of the onium salt compound represented by Formula (4A) or (4B) (onium salt compound (A)).

### (Evaluation Method for Residual Rate of Organic Cation Represented by Formula (2A))

The residual rate of the organic cation A in the onium salt compound represented by Formula (4A) or (4B) is preferably calculated by the following method.

In a case where the structure of the organic cation represented by Formula (2A) is unclear, the structure of the organic cation represented by Formula (2A) is specified by analyzing the onium salt compound represented by Formula (4A) or (4B) by a method such as ¹H NMR, ¹⁹F NMR, mass spectrometry, or elemental analysis, and then the following evaluation method is carried out. The anionic moiety does not affect the following evaluation methods regardless of the structure thereof.

In a case where the organic cation A is specified by the above-described analysis, the residual rate of the organic cation represented by Formula (2A) can be calculated by measuring ¹H NMR of a sample solution obtained by dissolving the onium salt compound (A) in a deuterated solvent, regardless of the corresponding anion moiety.

In addition, in the onium salt compound represented by Formula (4A) or (4B), in a case where it is confirmed by the above-described analysis that a trace amount of the organic cation represented by Formula (2A) is present, the residual rate can be calculated as a residual rate of the organic cation represented by Formula (2A) by the following method.

### (Evaluation Method for Residual Rate of Organic Cation Represented by Formula (2A))

The residual rate of the organic cation represented by Formula (2A) in the onium salt compound (A) is preferably calculated by the following method.

The ¹H NMR of a sample solution obtained by dissolving the onium salt compound (A) in a deuterated solvent is measured, and the ratio of the integral value of the peak A_{H} derived from the H atom of the onium salt compound (A) to the integral value of the peak 2_{H} derived from the organic cation represented by Formula (2A) is calculated. In a case where an integral value of the peak A_{H} per H atom is defined as I_{AH} and an integral value of the peak 2_{H} per H atom is defined as I_{2H}, the residual rate Z (mol %) of the organic cation represented by Formula (2A) can be calculated by Expression (3).

Z=I_{2H}/(I_{AH}+I_{2H})×100 (3)

However, in order to accurately detect a trace amount of the organic cation represented by Formula (2A), the concentration of the sample solution obtained by dissolving the onium salt compound (A) in the deuterated solvent is required to be 20% by weight or more. The concentration is more preferably 40% by weight or more.

The residual rate Z indicates the content (mol %) of the organic cation represented by Formula (2A) with respect to 1 mol of the onium salt compound represented by Formula (4A) or (4B) in the onium salt composition according to the embodiment of the present invention.

Since the onium salt having an organic cation represented by Formula (2A) does not have a function as a photoacid generator, in a case where the onium salt remains even in a trace amount, an increase in acid concentration fluctuation due to a decrease in effective acid cannot be ignored, particularly in the formation of an extremely fine pattern, and is likely to lead to deterioration in performance of LWR or the like. On the other hand, in a case where the hydrophilicity and hydrophobicity of the organic cation represented by Formula (2A) and the onium salt compound (A) are close to each other, it is difficult to purify the onium salt compound (A), and this causes a problem where the organic cation represented by Formula (2A) cannot be completely removed unless the purification is repeated multiple times, which lowers the production efficiency. As a result of intensive studies, it was found that an onium salt composition containing 0.001 mol % to 3 mol % of an organic cation represented by Formula (2A) with respect to 1 mol of the onium salt compound achieves both LWR performance and productivity.

In the onium salt composition according to the embodiment of the present invention, the content of the organic cation represented by Formula (2A) (hereinafter, also referred to as “organic cation A”) is preferably 0.001 mol % to 3 mol %, more preferably 0.001 mol % to 1.0 mol %, and still more preferably 0.001 mol % to 0.20 mol % with respect to 1 mol of the onium salt compound represented by Formula (4A) or (4B).

The method for producing the onium salt composition according to the embodiment of the present invention is not particularly limited, but the onium salt composition according to the embodiment of the present invention can be suitably produced, for example, by the above-described method for producing according to the embodiment of the present invention.

The organic cation represented by Formula (2A) is preferably represented by Formula (2B).

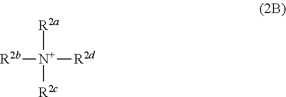

In Formula (2B), R^{2a} to R^{2d} each independently represent an alkyl group, a cycloalkyl group, or an aryl group.

As the alkyl group represented by R^{2a} to R^{2d}, a linear or branched alkyl group having 1 to 20 carbon atoms is preferable, and a linear alkyl group having 1 to 20 carbon atoms is more preferable.

Examples of the cycloalkyl group represented by R^{2a} to R^{2d} include a cycloalkyl group having 3 to 15 carbon atoms.

Examples of the aryl group represented by R^{2a} to R^{2d} include an aryl group having 6 to 14 carbon atoms, and a phenyl group is preferable.

The alkyl group, the cycloalkyl group, or the aryl group represented by R^{2a} to R^{2d} may have a substituent, and examples of the substituent include an alkyl group (for example, having 1 to 15 carbon atoms), a cycloalkyl group (for example, having 3 to 15 carbon atoms), an aryl group (for example, having 6 to 14 carbon atoms), an alkoxy group (for example, having 1 to 15 carbon atoms), and a cycloalkylalkoxy group (for example, having 1 to 15 carbon atoms).

R^{1a}, R^{1b}, and R^{1c} in Formula (4A) are each preferably an aryl group.

### [Actinic Ray-Sensitive or Radiation-Sensitive Film and Pattern Forming Method]

The actinic ray-sensitive or radiation-sensitive film formed of the composition of the embodiment of the present invention is preferably a resist film.

The procedure of the pattern forming method using the method for producing a composition according to the embodiment of the present invention is not particularly limited, but preferably has the following steps.
Step 0: a step of obtaining a composition by the method for producing a composition according to the embodiment of the present invention
Step 1: step of forming an actinic ray-sensitive or radiation-sensitive film on a substrate using the composition
Step 2: step of exposing the actinic ray-sensitive or radiation-sensitive film
Step 3: step of developing the exposed actinic ray-sensitive or radiation-sensitive film with a developer to form a pattern

Hereinafter, the procedure of each of the above-described steps will be described in detail.

### (Step 0: Step of Obtaining Composition by Method for Producing Composition According to Embodiment of Present Invention)

The step 0 is a step of obtaining a composition by the method for producing a composition according to the embodiment of the present invention. As described above, the method for producing the composition according to the embodiment of the present invention includes a step of mixing the onium salt compound (A) and the resin (B), but the composition according to the embodiment of the present invention may include further components in addition to the onium salt compound (A) and the resin (B).

### (Step 1: Actinic Ray-Sensitive or Radiation-Sensitive Film Forming Step)

The step 1 is a step of forming an actinic ray-sensitive or radiation-sensitive film on a substrate using the composition (the composition of the embodiment of the present invention).

Examples of the method of forming an actinic ray-sensitive or radiation-sensitive film on a substrate using the composition of the embodiment of the present invention include a method of applying the composition of the embodiment of the present invention onto a substrate.

It is preferable that the composition according to the embodiment of the present invention is filtered through a filter before the application, as necessary. A pore size of the filter is preferably 0.1 μm or less, more preferably 0.05 μm or less, and still more preferably 0.03 μm or less. The filter is preferably a polytetrafluoroethylene-made filter, a polyethylene-made filter, or a nylon-made filter.

The composition according to the embodiment of the present invention can be applied to a substrate (for example, silicon and silicon dioxide coating) as used in the manufacture of integrated circuit elements by a suitable application method such as an application using a spinner or a coater. The application method is preferably a spin coating using a spinner. A rotation speed upon the spin coating using a spinner is preferably 1000 to 3000 rotations per minute (rpm).

After the application of the composition according to the embodiment of the present invention, the substrate may be dried to form an actinic ray-sensitive or radiation-sensitive film. In addition, various underlying films (an inorganic film, an organic film, or an antireflection film) may be formed on an underlayer of the resist film.

Examples of the drying method include a method of heating and drying. The heating can be carried out using a unit included in an ordinary exposure machine and/or development machine, and may also be carried out using a hot plate or the like. A heating temperature is preferably 80° C. to 150° C., more preferably 80° C. to 140° C., and still more preferably 80° C. to 130° C. A heating time is preferably 30 to 1000 seconds, more preferably 60 to 800 seconds, and still more preferably 60 to 600 seconds.

A film thickness of the actinic ray or radiation-sensitive film is not particularly limited, but from the viewpoint that a fine pattern having higher accuracy can be formed, is preferably 10 to 120 nm. Among these, in a case of performing EUV exposure, the film thickness of the actinic ray or radiation-sensitive film is more preferably 10 to 65 nm and still more preferably 15 to 50 nm. In a case of performing ArF liquid immersion exposure, the film thickness of the actinic ray or radiation-sensitive film is more preferably 10 to 120 nm and still more preferably 15 to 90 nm.

A topcoat may be formed on an upper layer of the actinic ray-sensitive or radiation-sensitive film using a topcoat composition.

It is preferable that the topcoat composition is not mixed with the actinic ray-sensitive or radiation-sensitive film and can be uniformly applied onto the upper layer of the actinic ray-sensitive or radiation-sensitive film. The topcoat is not particularly limited, a topcoat known in the related art can be formed by the methods known in the related art, and for example, the topcoat can be formed based on the description in paragraphs [0072] to [0082] of JP2014-059543A.

For example, it is preferable that a topcoat including a basic compound as described in JP2013-61648A is formed on the actinic ray-sensitive or radiation-sensitive film. Specific examples of the basic compound which can be included in the topcoat include a basic compound which may be included in the composition of the embodiment of the present invention.

It is also preferable that the topcoat includes a compound which includes at least one group or bond selected from the group consisting of an ether bond, a thioether bond, a hydroxyl group, a thiol group, a carbonyl bond, and an ester bond.

### (Step 2: Exposing Step)

The step 2 is a step of exposing the actinic ray or radiation-sensitive film.

Examples of an exposing method include a method in which the formed actinic ray or radiation-sensitive film is irradiated with actinic ray or radiation through a predetermined mask.

Examples of the actinic ray or radiation include infrared light, visible light, ultraviolet light, far ultraviolet light, extreme ultraviolet light, X-rays, and electron beams, and the wavelength is preferably 250 nm or less and more preferably 220 nm or less. Far ultraviolet light having a wavelength of 1 to 200 nm, specifically, KrF excimer laser (248 nm), ArF excimer laser (193 nm), F₂ excimer laser (157 nm), EUV (13.5 nm), X-rays, and electron beams are particularly preferable.

The exposure amount of the actinic ray or radiation with which the actinic ray-sensitive or radiation-sensitive film is irradiated is not particularly limited, but is preferably an exposure amount that gives an appropriate decomposition rate of the photoacid generator by the irradiation with the actinic ray or radiation.

It is known that the photodecomposition reaction of the photoacid generator occurs according to Formula (XZ) as reported in the known literature (Proc. of SPIE Vol. 9425), and the amount of acid generated increases as the exposure amount is high and the decomposition rate of the photoacid generator is high, and the acid generation contrast (the slope of the decomposition rate of the photoacid generator with respect to the exposure amount) increases as the exposure amount is low and the decomposition rate of the photoacid generator is low.

Decomposition rate of photoacid generator=1−exp(-KE) . . . Formula (XZ)

In Formula (XZ), K represents a reaction rate constant, and E represents an exposure amount.

An exposure amount at which the decomposition rate of the photoacid generator contained in the actinic ray-sensitive or radiation-sensitive film is 1% to 99% is preferable, an exposure amount at which the decomposition rate of the photoacid generator is 10% to 90% is more preferable, and an exposure amount at which the decomposition rate of the photoacid generator is 20% to 80% is still more preferable.

The same content as described above is also described in the following non-patent document (Atsushi Sekiguchi et al.; Techniques for Measuring Rate Constants for Acid Generation from PAG (Photo Acid Generator) during ArF Exposure; International Conference of Photopolymer Science and Technology (ICPST-25); Jun. 25, 2008). The above non-patent document is also published in photonics technology journal “Light Edge”, No. 31 (October 2008).

It is preferable to perform baking (heating) before performing development and after the exposure. The baking accelerates a reaction in the exposed portion, and the sensitivity and the pattern shape are improved.

A heating temperature is preferably 80° C. to 150° C., more preferably 80° C. to 140° C., and still more preferably 80° C. to 130° C.

A heating time is preferably 10 to 1000 seconds, more preferably 10 to 180 seconds, and still more preferably 30 to 120 seconds.

The heating can be carried out using a unit included in an ordinary exposure machine and/or development machine, and may also be performed using a hot plate or the like.

This step is also referred to as a post-exposure baking.

### (Step 3: Developing Step)

The step 3 is a step of developing the exposed actinic ray-sensitive or radiation-sensitive film with a developer to form a pattern.

The developer may be either an alkali developer or a developer containing an organic solvent (hereinafter, also referred to as an organic developer).

Examples of a developing method include a method in which the substrate is immersed in a tank filled with a developer for a certain period of time (a dipping method), a method in which a development is performed by heaping a developer up onto the surface of the substrate by surface tension, and then leaving it to stand for a certain period of time (a puddle method), a method in which a developer is sprayed on the surface of the substrate (a spraying method), and a method in which a developer is continuously jetted onto the substrate rotating at a constant rate while scanning a developer jetting nozzle at a constant rate (a dynamic dispensing method).

In addition, after the step of performing the development, a step of stopping the development may be carried out while replacing the solvent with another solvent.

A developing time is not particularly limited as long as it is a period of time where the non-exposed portion of the resin is sufficiently dissolved, and is preferably 10 to 300 seconds and more preferably 20 to 120 seconds.

A temperature of the developer is preferably 0° C. to 50° C. and more preferably 15° C. to 35° C.

As the alkali developer, it is preferable to use an aqueous alkali solution including an alkali. The type of the aqueous alkali solution is not particularly limited, and examples thereof include an aqueous alkali solution including a quaternary ammonium salt typified by tetramethylammonium hydroxide, an inorganic alkali, a primary amine, a secondary amine, a tertiary amine, an alcoholamine, a cyclic amine, or the like. Among those, the aqueous solutions of the quaternary ammonium salts typified by tetramethylammonium hydroxide (TMAH) are preferable as the alkali developer. An appropriate amount of alcohols, a surfactant, or the like may be added to the alkali developer. The alkali concentration of the alkali developer is usually preferably 0.1% to 20% by mass. The pH of the alkali developer is usually preferably 10.0 to 15.0.

As the organic developer, a developer containing at least one organic solvent selected from the group consisting of a ketone-based solvent, an ester-based solvent, an alcohol-based solvent, an amide-based solvent, an ether-based solvent, and a hydrocarbon-based solvent is preferable.

A plurality of the above-described solvents may be mixed, or the solvent may be used in admixture with a solvent other than those described above or water. A moisture content in the entire developer is preferably less than 50% by mass, more preferably less than 20% by mass, and still more preferably less than 10% by mass, and it is particularly preferable that the entire developer contains substantially no water.

A content of the organic solvent with respect to the organic developer is preferably 50% by mass or more and 100% by mass or less, more preferably 80% by mass or more and 100% by mass or less, still more preferably 90% by mass or more and 100% by mass or less, and particularly preferably 95% by mass or more and 100% by mass or less, with respect to the total amount of the developer.

### (Other Steps)

It is preferable that the above-described pattern forming method includes a step of performing washing using a rinsing liquid after the step 3.

Examples of the rinsing liquid used in the rinsing step after the step of performing development using an alkali developer include pure water. An appropriate amount of a surfactant may be added to the pure water.

An appropriate amount of a surfactant may be added to the rinsing liquid.

The rinsing liquid used in the rinsing step after the developing step with an organic developer is not particularly limited as long as the rinsing liquid does not dissolve the pattern, and a solution including a common organic solvent can be used. As the rinsing liquid, a rinsing liquid containing at least one organic solvent selected from the group consisting of a hydrocarbon-based solvent, a ketone-based solvent, an ester-based solvent, an alcohol-based solvent, an amide-based solvent, and an ether-based solvent is preferably used.

A method for the rinsing step is not particularly limited, but examples thereof include a method in which a rinsing liquid is continuously jetted on a substrate rotated at a constant rate (a rotation application method), a method in which a substrate is dipped in a tank filled with a rinsing liquid for a certain period of time (a dip method), and a method in which a rinsing liquid is sprayed on a substrate surface (a spray method).

In addition, the pattern forming method may include a heating step (post bake) after the rinsing step. By this step, the developer and the rinsing liquid remaining between and inside the patterns are removed by baking. In addition, this step also has an effect that a resist pattern is annealed and the surface roughness of the pattern is improved. The heating step after the rinsing step is usually performed at 40° C. to 250° C. (preferably 90° C. to 200° C.) for usually 10 seconds to 3 minutes (preferably 30 seconds to 120 seconds).

In addition, an etching treatment on the substrate may be carried out using the formed pattern as a mask. That is, the substrate (or the underlayer film and the substrate) may be processed using the pattern formed in the step 3 as a mask to form a pattern on the substrate.

A method for processing the substrate (or the underlayer film and the substrate) is not particularly limited, but a method in which a pattern is formed on a substrate by subjecting the substrate (or the underlayer film and the substrate) to dry etching using the pattern formed in the step 3 as a mask is preferable. Oxygen plasma etching is preferable as the dry etching.

It is preferable that various materials (for example, a solvent, a developer, a rinsing liquid, a composition for forming an antireflection film, and a composition for forming a topcoat) used in the composition and the pattern forming method according to the embodiment of the present invention do not include impurities such as metals. The content of impurities contained in these materials is preferably 1 part per million (ppm) by mass or less, more preferably 10 parts per billion (ppb) by mass or less, still more preferably 100 parts per trillion (ppt) by mass or less, particularly preferably 10 parts per trillion (ppt) by mass or less, and most preferably 1 part per trillion (ppt) by mass or less. The lower limit thereof is not particularly limited, but is preferably 0 ppt by mass or more. Here, examples of the metal impurities include Na, K, Ca, Fe, Cu, Mg, Al, Li, Cr, Ni, Sn, Ag, As, Au, Ba, Cd, Co, Pb, Ti, V, W, and Zn.

Examples of a method for removing the impurities such as metals from the various materials include filtration using a filter. Details of the filtration using a filter are described in paragraph [0321] of WO2020/004306A.

Examples of a method for reducing the impurities such as metals included in the various materials include a method of selecting raw materials having a low content of metals as raw materials constituting the various materials, a method of subjecting raw materials constituting the various materials to filter filtration, and a method of performing distillation under the condition for suppressing the contamination as much as possible by, for example, lining the inside of a device with TEFLON (registered trademark).

In addition to the filter filtration, removal of the impurities by an adsorbing material may be performed, or a combination of filter filtration and an adsorbing material may be used. As the adsorbing material, known adsorbing materials can be used, and for example, inorganic adsorbing materials such as silica gel and zeolite and organic adsorbing materials such as activated carbon can be used. It is necessary to prevent the incorporation of impurities such as metals in the production process in order to reduce the metal impurities included in the above-described various materials. Sufficient removal of the metal impurities from a production device can be confirmed by measuring the content of metal components included in a washing solution used to wash the production device. A content of the metal components included in the washing solution after the use is preferably 100 parts per trillion (ppt) by mass or less, more preferably 10 ppt by mass or less, and still more preferably 1 ppt by mass or less. The lower limit thereof is not particularly limited, but is preferably 0 ppt by mass or more.

A conductive compound may be added to an organic treatment liquid such as the rinsing liquid in order to prevent breakdown of chemical liquid pipes and various parts (a filter, an O-ring, a tube, and the like) due to electrostatic charging, and subsequently generated electrostatic discharging. The conductive compound is not particularly limited, and examples thereof include methanol. An addition amount is not particularly limited, but from the viewpoint that preferred development characteristics or rinsing characteristics are maintained, the addition amount is preferably 10% by mass or less and more preferably 5% by mass or less. The lower limit thereof is not particularly limited, but is preferably 0.01% by mass or more.

For members of the chemical liquid pipe, for example, various pipes coated with stainless steel (SUS), or a polyethylene, polypropylene, or a fluororesin (a polytetrafluoroethylene resin, a perfluoroalkoxy resin, or the like) that has been subjected to an antistatic treatment can be used. In the same manner, for the filter or the O-ring, polyethylene, polypropylene, or a fluororesin (polytetrafluoroethylene, a perfluoroalkoxy resin, or the like) that has been subjected to an antistatic treatment can be used.

### [Method for Manufacturing Electronic Device]

The present specification also relates to a method for manufacturing an electronic device, which includes the above-described pattern forming method, and an electronic device manufactured by the method for manufacturing.

Examples of a suitable aspect of the electronic device according to the present specification include an aspect of being mounted on electric and electronic apparatus (for example, home appliances, office automation (OA)-related equipment, media-related equipment, optical equipment, telecommunication equipment, and the like).

### EXAMPLES

Hereinbelow, the present invention will be described in more detail with reference to Examples. The materials, the amounts of materials used, the proportions, the treatment details, and the treatment procedure in Examples below may be appropriately modified as long as the modifications do not depart from the spirit of the present invention. Therefore, the scope of the present invention should not be construed as being limited to Examples shown below.

### Example S1: Synthesis of Compound A1

### Synthesis of Intermediate Compound (3-1)

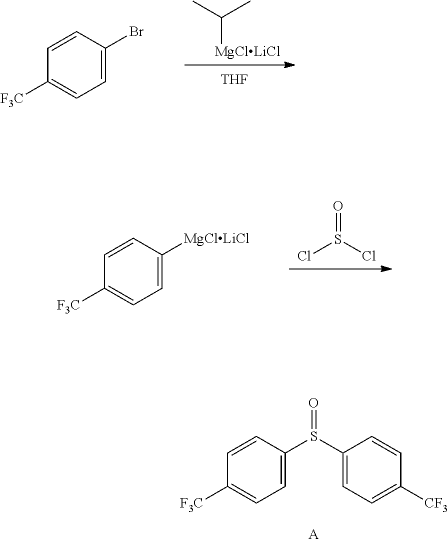

A mixture of 160 mL of a 1.3 M tetrahydrofuran (THF) solution of isopropylmagnesium chloride-lithium chloride complex and 40 mL of tetrahydrofuran was cooled to 0° C., and 45.4 g of 1-bromo-4-(trifluoromethyl)benzene was added dropwise thereto. The obtained mixture was stirred at room temperature for 6 hours to prepare a Grignard reagent. The mixture was cooled to 0° C., and 12.0 g of thionyl chloride was slowly added dropwise thereto. 160 mL of 1 N hydrochloric acid was added dropwise thereto, and the mixture was extracted with 80 mL of heptane. An organic layer was washed with 200 mL of water three times, and the organic layer was concentrated under reduced pressure. 100 mL of heptane was added to the obtained crude product, and the mixture was stirred and filtered to obtain a compound A with a yield of 60%.

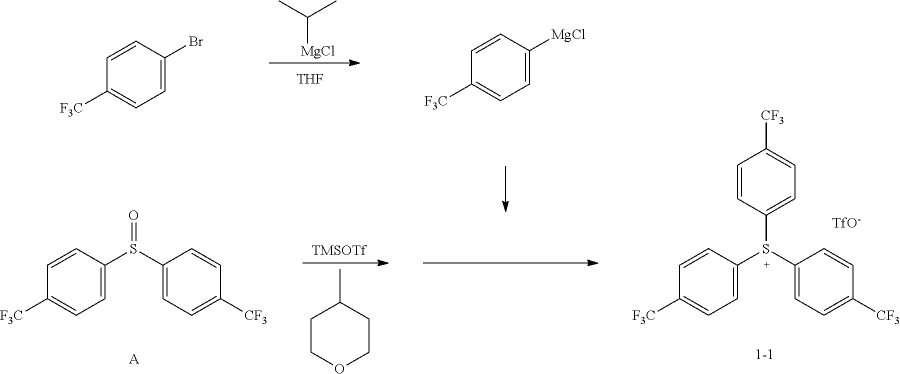

120 mL of a 1.0 M tetrahydrofuran (THF) solution of isopropylmagnesium chloride was cooled to 0° C., and 25.2 g of 1-bromo-4-(trifluoromethyl)benzene was added dropwise thereto. The obtained mixture was stirred at room temperature for 6 hours to prepare a Grignard reagent. 19.0 g of the compound A was dissolved in 86 mL of 4-methyltetrahydropyran, the solution was cooled to 0° C., and 62.4 g of trimethylsilyl trifluoromethanesulfonate (TMSOTf) was added dropwise thereto. The prepared Grignard reagent was added dropwise to the obtained mixture, and the mixture was stirred for 1 hour. After 86 mL of 1 N hydrochloric acid was added dropwise, extraction was carried out with 140 mL of methylene chloride. The organic layer was washed with 140 mL of water three times, and the organic layer was concentrated under reduced pressure. 200 mL of diisopropyl ether was added to the obtained crude product, and the mixture was stirred and filtered to obtain a compound (1-1) with a yield of 67%.

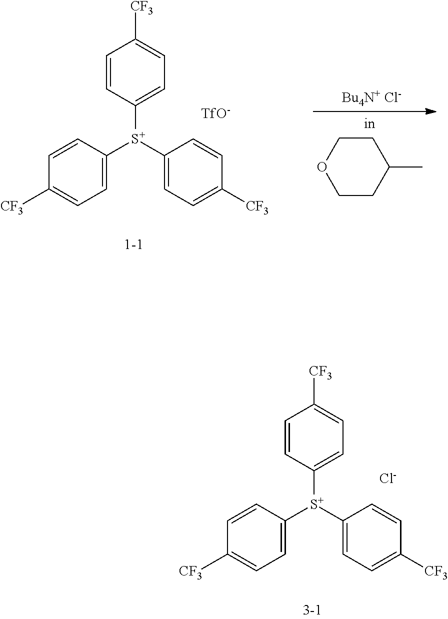

10 g of the compound (1-1) was dissolved in 120 g of 4-methyltetrahydropyrane, tetrabutylammonium chloride (4.2 g) was added thereto while stirring, the mixture was heated to 40° C., and the mixture was further stirred for 2 hours. After returning to room temperature (25° C.), the mixture was filtered to collect crude crystals. The crude crystals were dissolved in 21 g of acetone, 105 g of tert-butyl methyl ether was added thereto while stirring at 40° C., and crystals were precipitated. After returning to room temperature, the mixture was filtered to collect crystals, thereby obtaining a compound (3-1) with a yield of 71%.
¹H NMR (Acetone-d6): 8.72, 8.07 ppm
¹⁹F NMR(Acetone-d6): −63.8 ppm
TfO⁻ represents CF₃SO₃⁻.

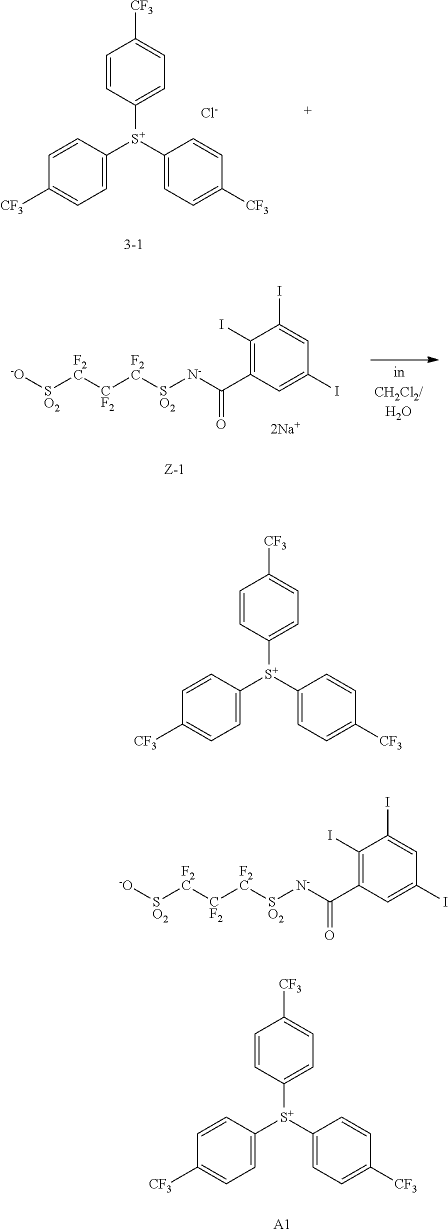

15 g (17.9 mmol) of the compound (Z-1), 250 g of methylene chloride, and 200 g of water were mixed, and then 18 g (35.8 mmol) of the compound (3-1) was added thereto, followed by stirring at room temperature for 30 minutes. After removing the aqueous layer, the organic layer was washed once with 0.1 N hydrochloric acid and three times with water, and the organic layer was concentrated under reduced pressure. 100 g of diisopropyl ether was added to the obtained crude product, the mixture was stirred, and the filtered solid was dried under reduced pressure for 5 hours to obtain a compound A1 with a yield of 87%.
¹H NMR (Acetone-d6): 8.37, 8.16, 8.11, 7.61 ppm
¹⁹F NMR(Acetone-d6): −63.8, −112.7, −114.4, −118.6 ppm

### Examples S2 to S26: Synthesis of Compounds A2 to A26

Compounds A2 to A26 shown in Tables 1 to 3 were obtained by the same method as in Example S1, except that the onium salt compound (1), the salt compound (2), and the non-aqueous solvent (S) were changed to the compounds shown in Tables 1 to 3, and the salt compound (5) was changed to the compound shown in Tables 1 to 3.

### Comparative Example RS1: Synthesis of Compound RA1

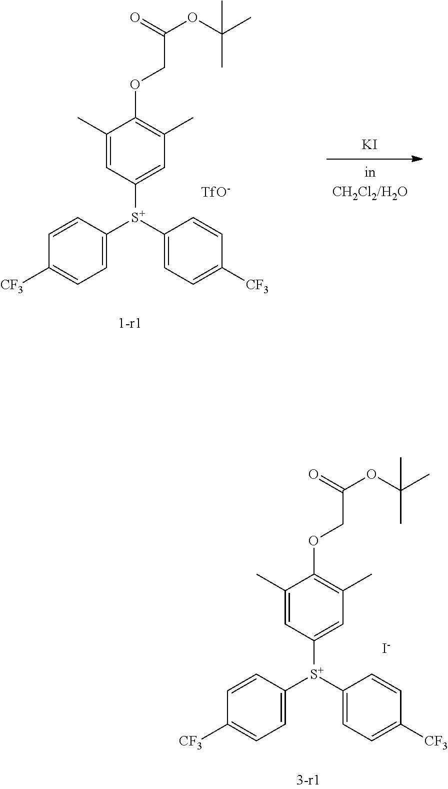

An intermediate compound (3-r1) was synthesized in the same manner as in JP2014-97969A, except that the compound (1-r1) was used.

A compound RA1 was obtained by the same method as in Example S1, except that the intermediate compound (3-r1) was used as the onium salt compound (3), and the salt compound (5) was changed to the salt compound consisting of the anion contained in the onium salt compound (A) and the sodium cation, which are shown in Table 2.

### Comparative Example RS2: Synthesis of Intermediate Compound (3-r3)

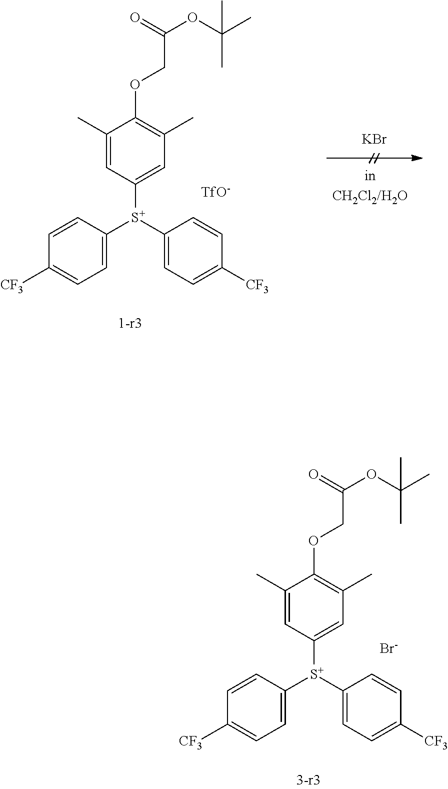

11.1 g of the compound (1-r3) was dissolved in 125 ml of methylene chloride, 125 ml of a 1 M potassium bromide aqueous solution was added thereto, and the mixture was stirred at room temperature for 30 minutes. After removing the aqueous layer by a liquid separation operation, the mixture was washed with 125 ml of a 1 M potassium bromide aqueous solution three times and with 125 ml of water four times. As a result of confirming the NMR of the residue after concentration under reduced pressure, only the peak of the compound (1-r3) was detected, and the compound (3-r3) could not be obtained.

### [Evaluation]

The obtained compounds A2 to A26 and RA1 were evaluated as shown below. The evaluation results are listed in Tables 1 to 3.

### <Evaluation of Residual Rate of Onium Salt Compound (1)>

¹⁹F NMR of a sample solution obtained by dissolving 0.2 g of the onium salt compound (A) in 0.8 g of heavy acetone (heavy methanol in the case of low solubility in heavy acetone) was measured, and the residual rate Y (mol %) of the onium salt compound (1) was calculated using Expression (2-1). For the compound A15 and the compound A19, 1,4-difluorobenzene (0.5 mol with respect to 1 mol of the onium salt compound (A)) was added as an internal standard substance P to the sample solution, ¹H NMR and ¹⁹F NMR were measured, and the residual rate Y (mol %) of the onium salt compound (1) was calculated using the above-described Formula (2-2).

### <Evaluation of Residual Rate of Onium Salt Compound (3)>

A commercially available 0.01 N silver nitrate aqueous solution was diluted 10 times with ion exchange water to prepare a 0.001 N silver nitrate aqueous solution. 100 mg of the onium salt compound (A) is weighed and dissolved in 60 ml of a mixed solvent of THF and water (THF/water=54/6 (volume ratio)) to prepare a sample solution. Using the 0.001 N silver nitrate aqueous solution prepared above, the amount of the titrant was measured with an automatic titrator (AT-510, manufactured by KYOTO ELECTRONICS MANUFACTURING Co., Ltd.) for the empty solution of only the mixed solvent and the sample solution. From the results of the obtained amount of the titrant, the residual rate X (mol %) of the onium salt compound (3) with respect to 1 mol of the onium salt compound (A) was calculated using Expression (1) described above.

### <Calculation of SP Value of Non-Aqueous Solvent (S)>

The calculation was performed using calculation software HSPiP 5th Edition 5.1.08.

The values are shown in Tables 1 to 3.

TABLE 1

| | | | | | | | Onium salt compound (A) | | |
|---|---|---|---|---|---|---|---|---|---|
| | | | | | | | Chemical | Residual | |
| | | | | | Calculated | | foumula of | rate Y of | Residual rate |
| | Onium salt | Salt | Onium salt | | SP value of | Salt | onium salt | onium salt | X of onium salt |
| | compound | compound | compound | Non-aqueous | non-aqueous | compound | compound | compound | compound |
| | (1) | (2) | (3) | solvent (S) | solvent (S) | (5) | (A) | (1) (mol %) | (3) (mol %) |
| Example | (1)-1 | (2)-1 | (3)-1 | (S)-1 | 17.8 | (5)-1 | A1 | | |
| S1 | | | | | | | (A)-1 | 0.1 | <0.1 |
| Example | (1)-1 | (2)-1 | (3)-1 | (S)-2 | 21.5 | (5)-1 | A2 | | |
| S2 | | | | | | | (A)-1 | 0.9 | <0.1 |
| Example | (1)-1 | (2)-1 | (3)-1 | (S)-3 | 17.7 | (5)-2 | A3 | | |
| S3 | | | | | | | (A)-2 | 0.1 | <0.1 |
| Example | (1)-2 | (2)-1 | (3)-2 | (S)-1 | 17.8 | (5)-3 | A4 | | |
| S4 | | | | | | | (A)-3 | 0.1 | <0.1 |
| Example | (1)-2 | (2)-2 | (3)-2 | (S)-1 | 17.8 | (5)-3 | A5 | | |
| S5 | | | | | | | (A)-3 | 1.2 | <0.1 |
| Example | (1)-2 | (2)-3 | (3)-2 | (S)-4 | 17.7 | (5)-4 | A6 | | |
| S6 | | | | | | | (A)-4 | 0.3 | <0.1 |
| Example | (1)-3 | (2)-4 | (3)-2 | (S)-5 | 17.1 | (5)-5 | A7 | | |
| S7 | | | | | | | (A)-5 | 0.3 | <0.1 |
| Example | (1)-4 | (2)-5 | (3)-3 | (S)-6 | 17.0 | (5)-6 | A8 | | |
| S8 | | | | | | | (A)-6 | 0.2 | 0.1 |
| Example | (1)-4 | (2)-1 | (3)-4 | (S)-3 | 17.7 | (5)-7 | A9 | | |
| S9 | | | | | | | (A)-7 | 0.1 | <0.1 |
| Example | (1)-5 | (2)-6 | (3)-5 | (S)-4 | 17.7 | (5)-8 | A10 | | |
| S10 | | | | | | | (A)-8 | 0.3 | <0.1 |
| Example | (1)-6 | (2)-7 | (3)-6 | (S)-1 | 17.8 | (5)-9 | A11 | | |
| S11 | | | | | | | (A)-9 | 0.1 | <0.1 |
| Example | (1)-7 | (2)-6 | (3)-7 | (S)-7 | 17.5 | (5)-10 | A12 | | |
| S12 | | | | | | | (A)-10 | 0.2 | <0.1 |
| Example | (1)-7 | (2)-8 | (3)-8 | (S)-6 | 17.0 | (5)-11 | A13 | | |
| S13 | | | | | | | (A)-11 | 0.3 | 0.2 |
| Example | (1)-8 | (2)-8 | (3)-9 | (S)-3 | 17.7 | (5)-12 | A14 | | |
| S14 | | | | | | | (A)-12 | 0.9 | 0.1 |
| Example | (1)-9 | (2)-9 | (3)-10 | (S)-1 | 17.8 | (5)-13 | A15 | | |
| S15 | | | | | | | (A)-13 | 1.8 | <0.1 |

TABLE 2

| | | | | | | | Onium salt compound (A) | | |
|---|---|---|---|---|---|---|---|---|---|
| | | | | | | | Chemical | Residual | Residual |
| | | | | | Calculated SP | | foumula of | rate Y of | rate X of |
| | Onium salt | Salt | | | value of | Salt | onium salt | onium salt | onium salt |
| | compound | compound | Onium salt | Non-aqueous | non-aqueous | compound | compound | compound | compound |
| | (1) | (2) | compound (3) | solvent (S) | solvent (S) | (5) | (A) | (1) (mol %) | (3) (mol %) |
| Example S16 | (1)-10 | (2)-8 | (3)-11 | (S)-3 | 17.7 | (5)-14 | A16 | | |
| | | | | | | | (A)-14 | 0.2 | 0.1 |
| Example S17 | (1)-11 | (2)-10 | (3)-12 | (S)-9 | 18.3 | (5)-15 | A17 | | |
| | | | | | | | (A)-15 | 0.2 | <0.1 |
| Example S18 | (1)-12 | (2)-11 | (3)-13 | (S)-8 | 17.5 | (5)-16 | A18 | | |
| | | | | | | | (A)-16 | 1.1 | <0.1 |
| Example S19 | (1)-13 | (2)-3 | (3)-14 | CHCl₃ | 19.5 | (5)-17 | A19 | | |
| | | | | | | | (A)-17 | 1.5 | <0.1 |
| Example S20 | (1)-14 | (2)-1 | (3)-15 | (S)-3 | 17.7 | (5)-18 | A20 | | |
| | | | | | | | (A)-18 | 0.1 | <0.1 |
| Example S21 | (1)-15 | (2)-1 | (3)-16 | CH₃CN | 24.3 | (5)-10 | A21 | | |
| | | | | | | | (A)-19 | 1.3 | <0.1 |
| Example S22 | (1)-4 | (2)-7 | (3)-4 | (S)-7/(S)-5 = | 17.1 | (5)-6 | A22 | | |
| | | | | 1:1 (mass ratio) | | | (A)-6 | 0.2 | 0.1 |
| Comparative | (1)-2 | KI | (3)-R1 | CH₂Cl₂/ | — | (5)-3 | RA1 | | |
| Example RS1 | | | | H₂O | | | (A)-3 | 0.5 | 3.1 |
| Comparative | (1)-2 | KBr | Synthesis | CH₂Cl₂/ | — | — | — | | |
| Example RS2 | | | unavailable | H₂O | | | Synthesis | — | — |
| | | | | | | | unavailable | | |

TABLE 3

| | | | | | | | Onium salt compound (A) | | |
|---|---|---|---|---|---|---|---|---|---|
| | | | | | | | Chemical | Residual | Residual |
| | | | | | Calculated | | foumula of | rate Y of | rate X of |
| | Onium salt | Salt | Onium salt | | SP value of | Salt | onium salt | onium salt | onium salt |
| | compound | compound | compound | Non-aqueous | non-aqueous | compound | compound | compound | compound |
| | (1) | (2) | (3) | solvent (S) | solvent (S) | (5) | (A) | (1) (mol %) | (3) (mol %) |
| Example | (1)-11 | (2)-10 | (3)-12 | (S)-9 | 18.3 | (5)-19 | A23 | | |
| S23 | | | | | | | (A)-20 | 0.2 | <0.1 |
| Example | (1)-11 | (2)-10 | (3)-12 | (S)-9 | 18.3 | (5)-20 | A24 | | |
| S24 | | | | | | | (A)-21 | 0.2 | <0.1 |
| Example | (1)-11 | (2)-10 | (3)-12 | (S)-9 | 18.3 | (5)-21 | A25 | | |
| S25 | | | | | | | (A)-22 | 0.2 | <0.1 |
| Example | (1)-11 | (2)-10 | (3)-12 | (S)-9 | 18.3 | (5)-22 | A26 | | |
| S26 | | | | | | | (A)-23 | 0.2 | <0.1 |

The structures of the “Onium salt compound (1)” described in Tables 1 to 3 are described below.

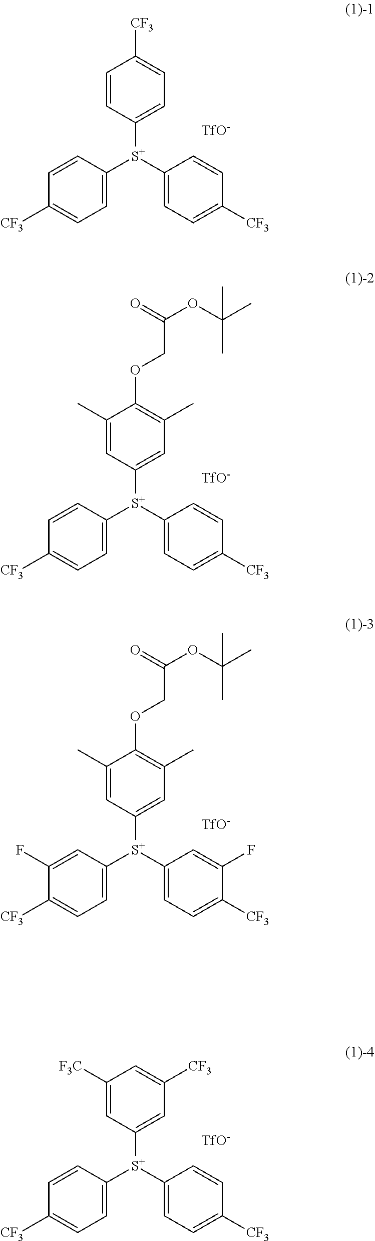

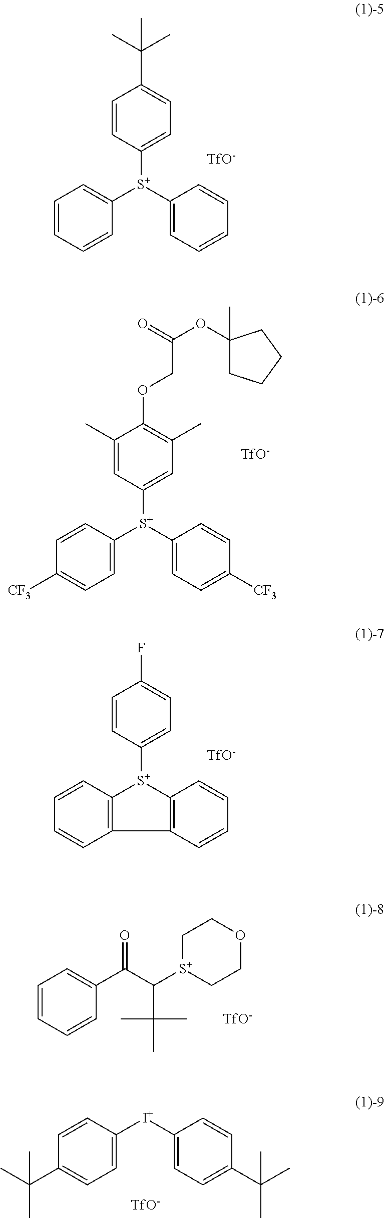

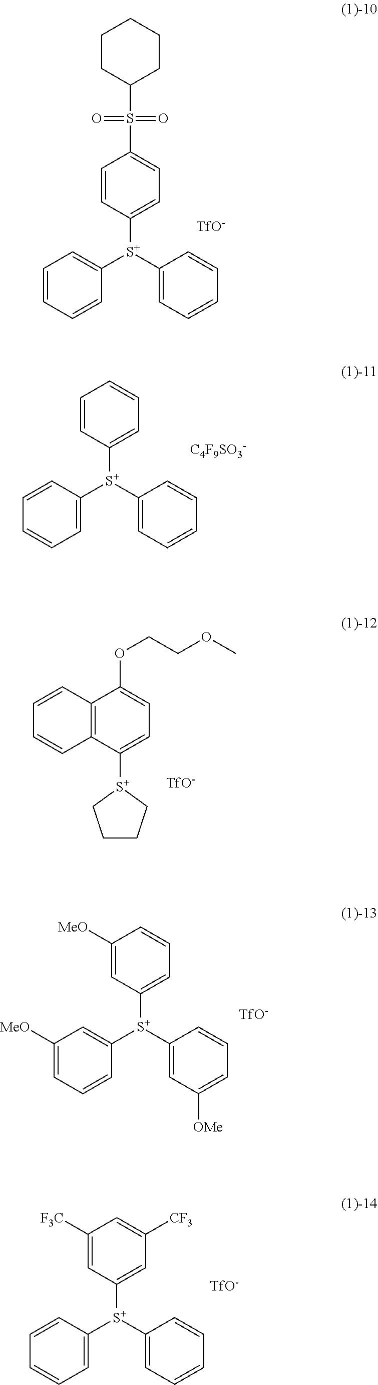

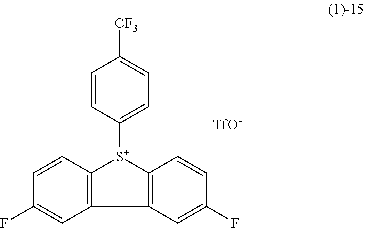

The structures of the “Salt compounds (2)” described in Tables 1 to 3 are described below. It is noted that KI and KBr are not the salt compound (2), but are described in the section of “Salt compound (2)” for convenience.

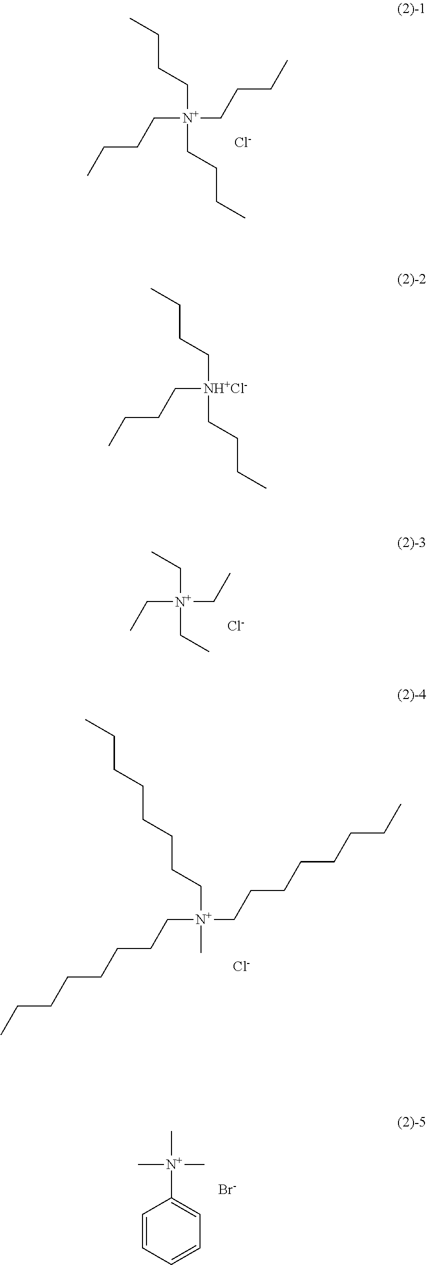

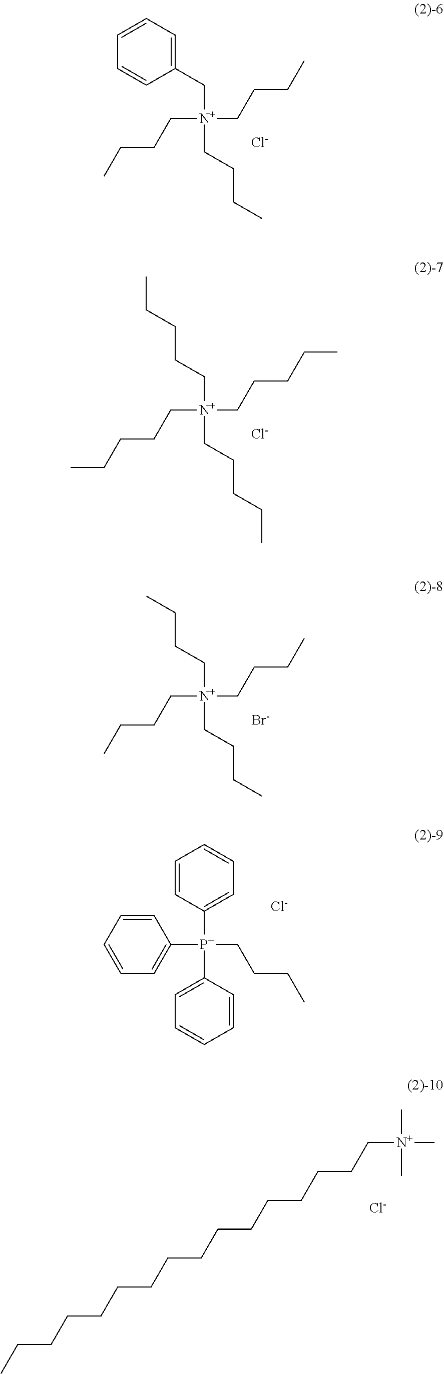

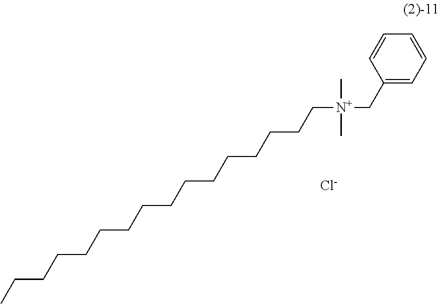 [SMILES: CCCCCCCCCCCCCCCC[N+](C)(C)Cc1ccccc1 ; [Cl-]]

The structures of the “Onium salt compound (3)” described in Tables 1 to 3 are described below.

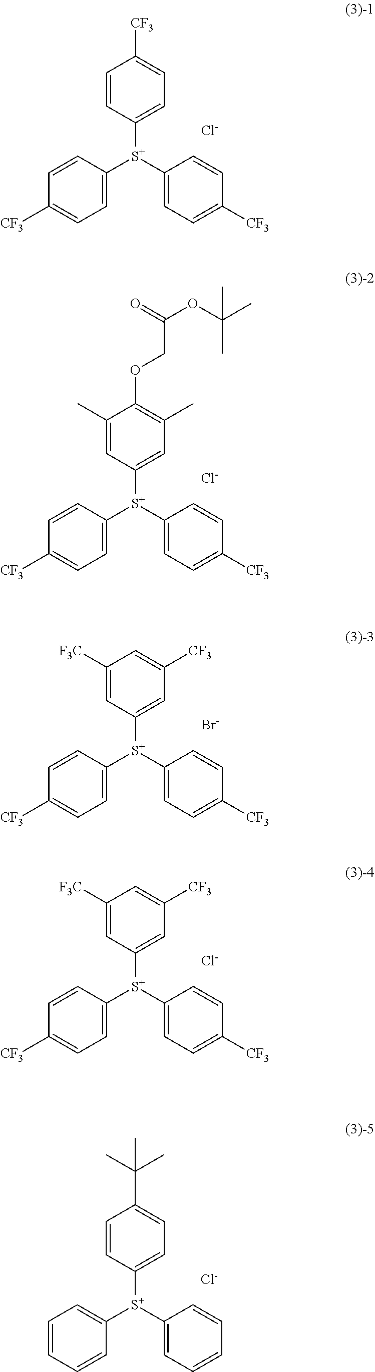

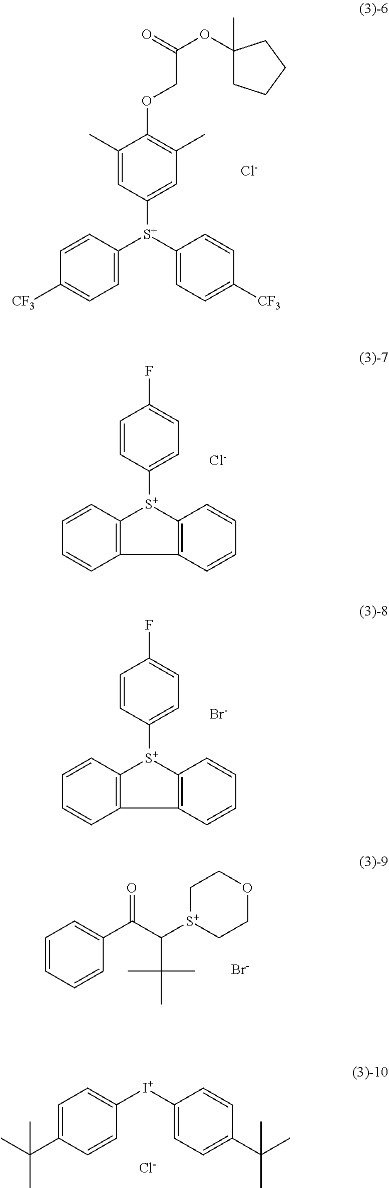

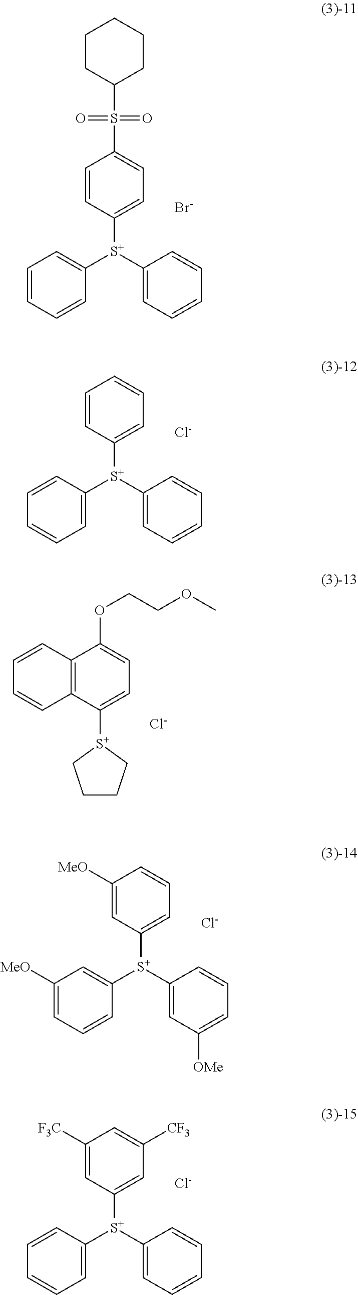

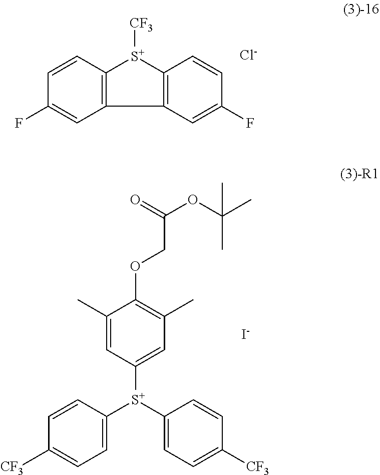 [SMILES: Fc1ccc2c(c1)c1cc(F)ccc1[s+]2C(F)(F)F ; [Cl-] ; Cc1cc([S+](c2ccc(C(F)(F)F)cc2)c2ccc(C(F)(F)F)cc2)cc(C)c1OCC(=O)OC(C)(C)C ; [I-]]

The compound (3)-R1 described in “Onium salt compound (3)” in Comparative Example RS1 described in Table 2 is not the onium salt compound (3), but is described as “Onium salt compound (3)” for convenience.

The structures of the “Non-aqueous solvent (S)” described in Tables 1 to 3 are described below.

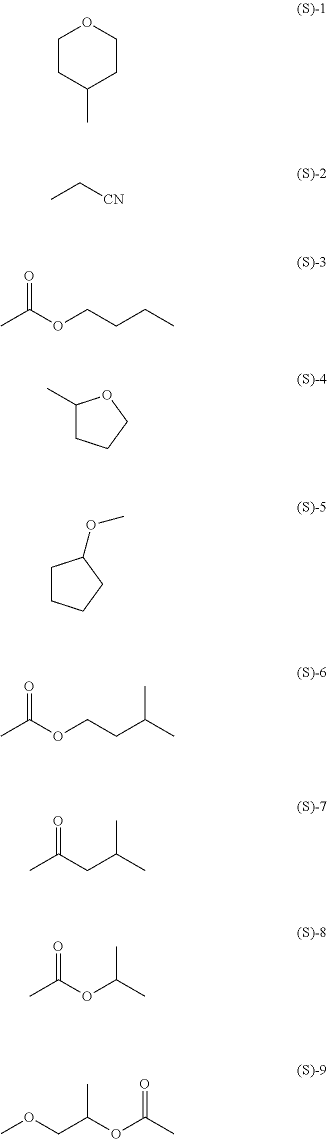

The structures of the “Salt compounds (5)” described in Tables 1 to 3 are described below.

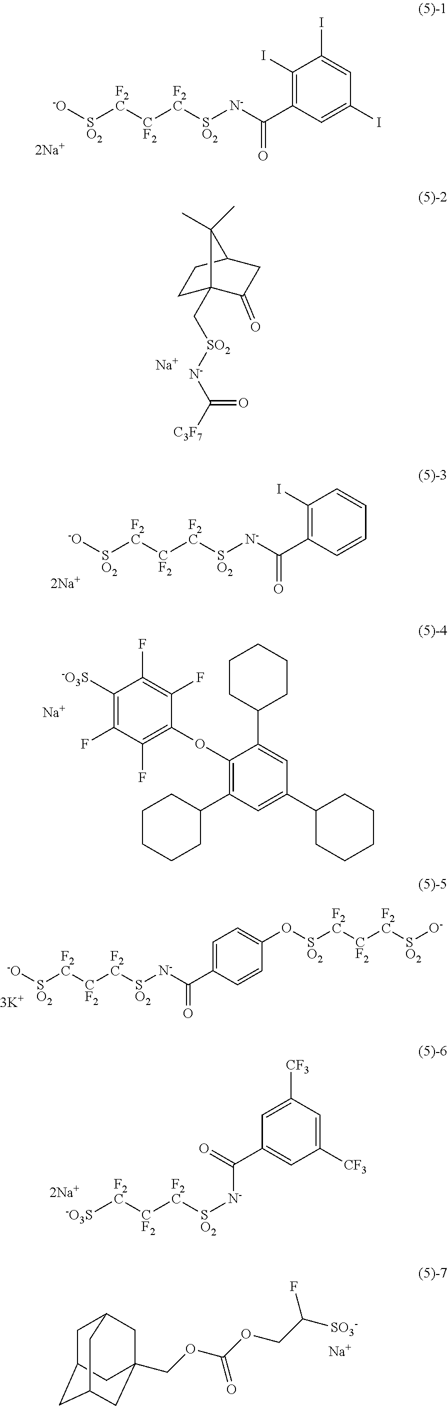

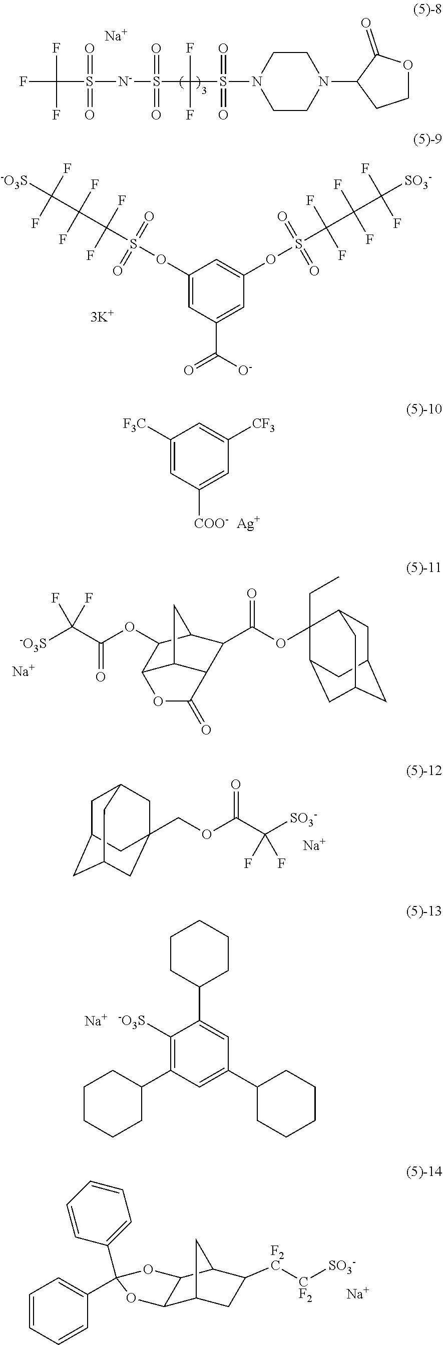

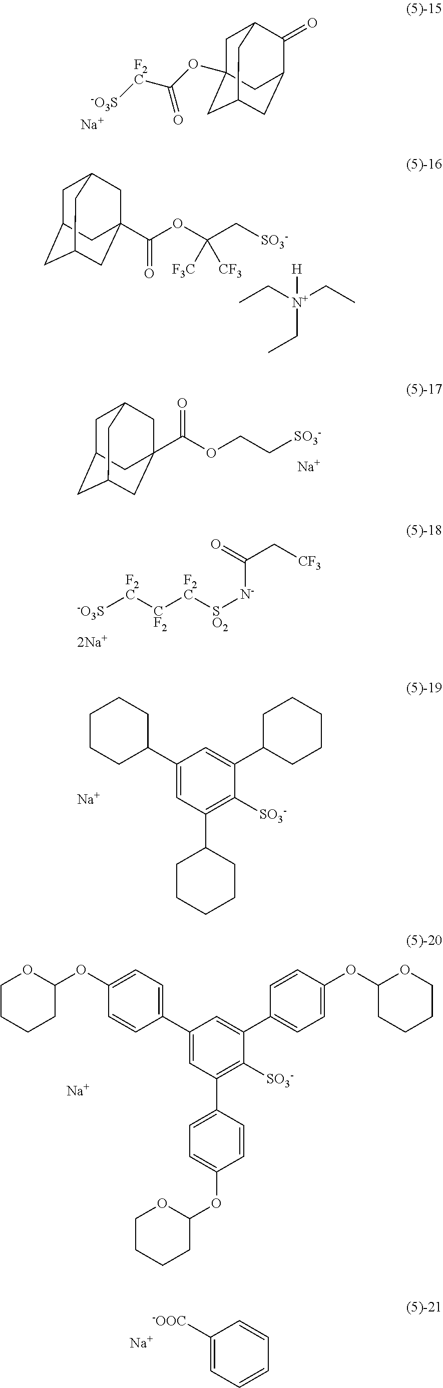

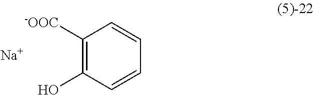

The structures of the “Chemical formula of onium salt compound (A)” described in Tables 1 to 3 are described below.

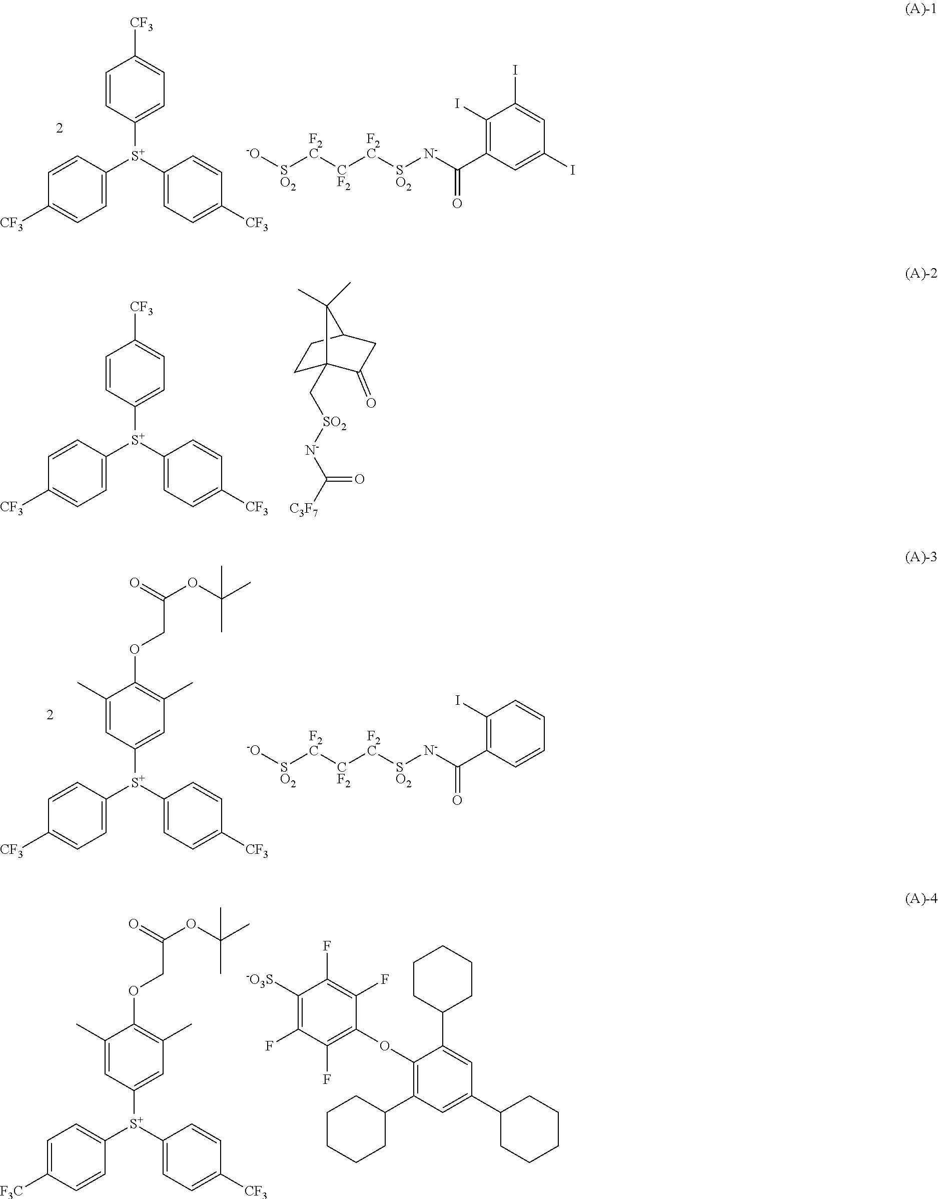

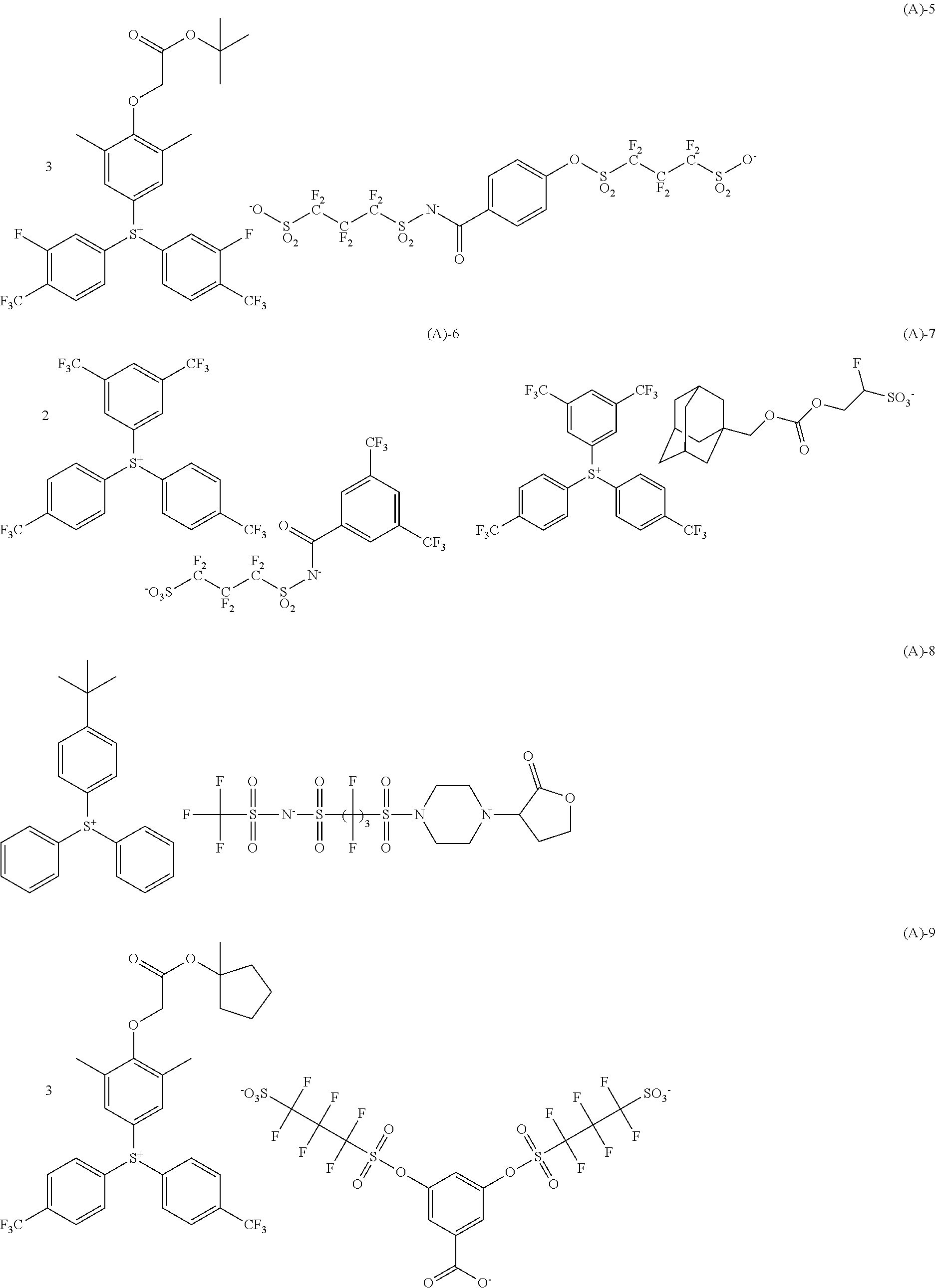

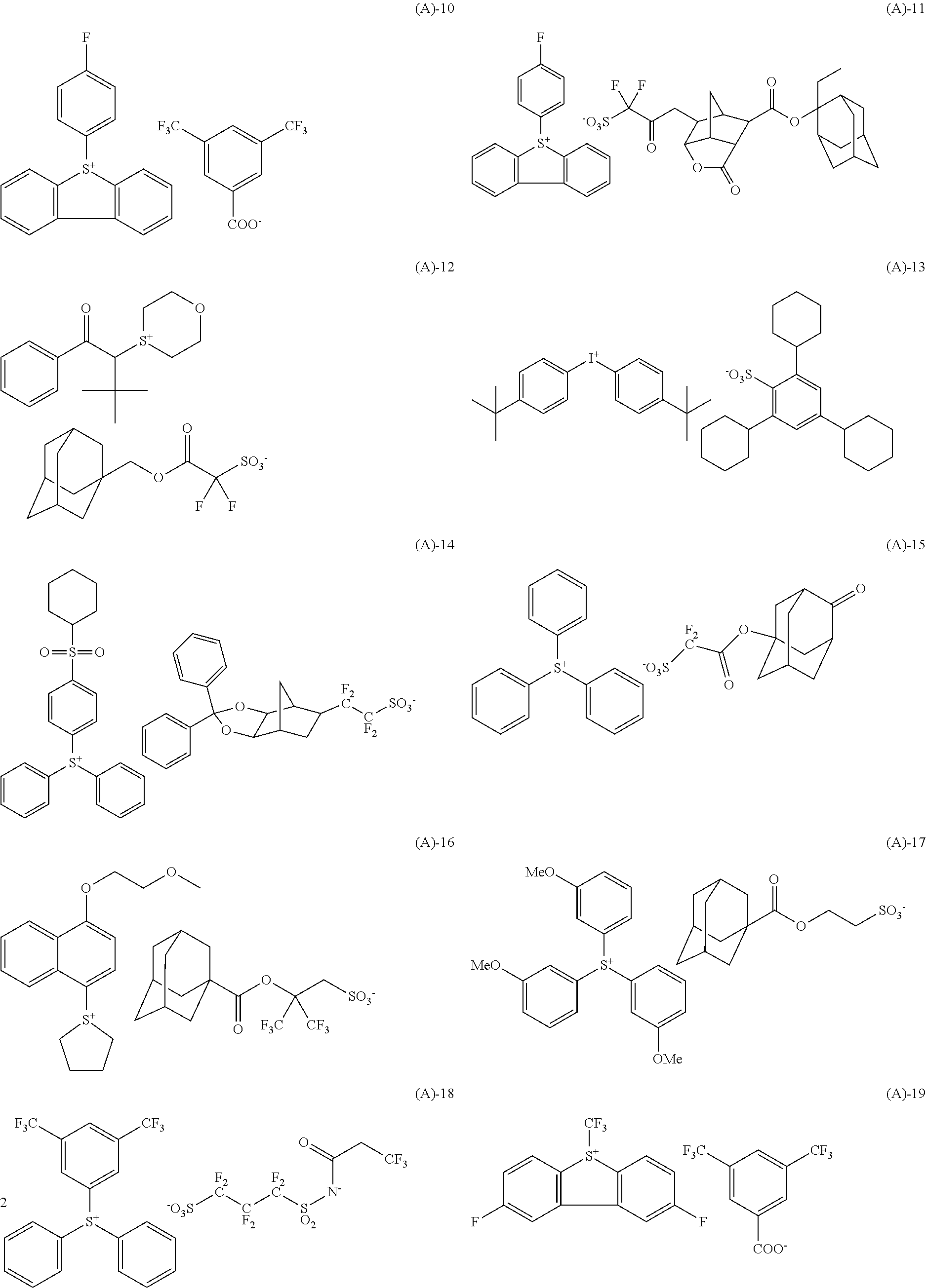

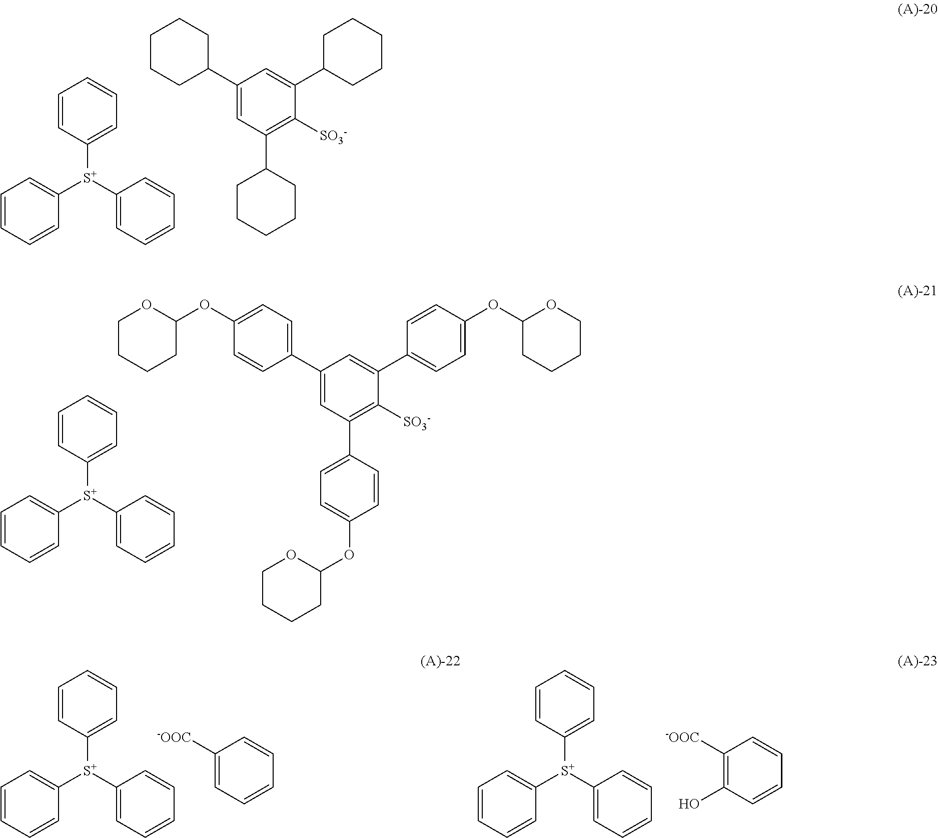

In the compounds A1 to A26 synthesized by the method for producing an onium salt compound according to the embodiment of the present invention, the residual amounts of the onium salt compound (1) and the onium salt compound (3) were 2 mol % or less, and the residual amounts of the raw material compound and the intermediate compound were small. On the other hand, it was found that a large amount of the onium salt compound (3) remained in the compound RA1.

### <Onium Salt Composition>

The compounds A1 to A26 and the compound RA1, which are onium salt compounds, were respectively set as onium salt compositions N1 to N26 and NR1.

The compound RA1 was subjected to ¹H NMR measurement, but the cation represented by Formula (2A) could not be confirmed.

### Reference Example RS3: Synthesis of Compound RA2

A compound RA2 was obtained by the same method as in Example S1, except that 3.5 mol % of the following compound was added to the compound (Z-1). The compound RA2 which is an onium salt compound was used as an onium salt composition NR2. Bu represents an n-butyl group.

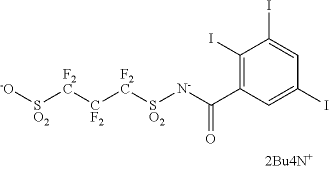

### <Evaluation of Residual Rate of Organic Cation Represented by Formula (2A)>

¹H NMR of a sample solution obtained by dissolving 0.2 g of the onium salt compound (A) in 0.8 g of heavy acetone (heavy methanol in the case of low solubility in heavy acetone) was measured, and the residual rate Z (mol %) was calculated using the above-described Expression (3).

The structures of the organic cations represented by Formula (2A) in each of the onium salt compositions are shown in Tables 4 and 5. The amount (mol %) of the organic cation represented by Formula (2A) with respect to 1 mol of the onium salt compound is shown in Tables 4 and 5 as “Residual rate Z (mol %) of cation represented by Formula (2A)”.

TABLE 4

| | | | | | Residual rate |
|---|---|---|---|---|---|
| | | | Onium | Organic | Z of cation |
| | Onium | | salt | cation | represented |
| | salt | | com- | represented | by Formula |
| | com- | Com- | pound | by Formula | (2A) |
| | pound | pound | (A) | (2A) | (mol %) |
| Example T1 | N1 | A1 | A1 | (Q)-1 | 0.008 |
| Example T2 | N2 | A2 | A2 | (Q)-1 | 0.006 |
| Example T3 | N3 | A3 | A3 | (Q)-1 | 0.006 |
| Example T4 | N4 | A4 | A4 | (Q)-1 | 0.004 |
| Example T5 | N5 | A5 | A5 | (Q)-2 | 0.010 |
| Example T6 | N6 | A6 | A6 | (Q)-3 | 0.005 |
| Example T7 | N7 | A7 | A7 | (Q)-4 | 0.004 |
| Example T8 | N8 | A8 | A8 | (Q)-5 | 0.009 |
| Example T9 | N9 | A9 | A9 | (Q)-1 | 0.008 |
| Example T10 | N10 | A10 | A10 | (Q)-6 | 0.008 |
| Example T11 | N11 | A11 | A11 | (Q)-7 | 0.007 |
| Example T12 | N12 | A12 | A12 | (Q)-6 | 0.006 |
| Example T13 | N13 | A13 | A13 | (Q)-1 | 0.006 |
| Example T14 | N14 | A14 | A14 | (Q)-1 | 0.008 |
| Example T15 | N15 | A15 | A15 | (Q)-8 | 0.010 |
| Example T16 | N16 | A16 | A16 | (Q)-1 | 0.005 |
| Example T17 | N17 | A17 | A17 | (Q)-9 | 0.009 |
| Example T18 | N18 | A18 | A18 | (Q)-10 | 0.008 |
| Example T19 | N19 | A19 | A19 | (Q)-3 | 0.004 |
| Example T20 | N20 | A20 | A20 | (Q)-1 | 0.004 |
| Example T21 | N21 | A21 | A21 | (Q)-1 | 0.006 |
| Example T22 | N22 | A22 | A22 | (Q)-7 | 0.006 |
| Comparative | NR1 | RA1 | RA1 | — | — |
| Example RS1 | | | | | |
| Reference | NR2 | RA2 | RA2 | (Q)-1 | 3.5 |
| Example RS3 | | | | | |

TABLE 5

| | | | | | Residual rate |
|---|---|---|---|---|---|
| | | | Onium | Organic | Z of cation |
| | Onium | | salt | cation | represented |
| | salt | | com- | represented | by Formula |
| | com- | Com- | pound | by Formula | (2A) |
| | pound | pound | (A) | (2A) | (mol %) |
| Example T23 | N23 | A23 | A23 | (Q)-9 | 0.009 |
| Example T24 | N24 | A24 | A24 | (Q)-9 | 0.009 |
| Example T25 | N25 | A25 | A25 | (Q)-9 | 0.009 |
| Example T26 | N26 | A26 | A26 | (Q)-9 | 0.009 |

The structures of the “Cation represented by Formula (2A)” described in Tables 4 and 5 are described below.

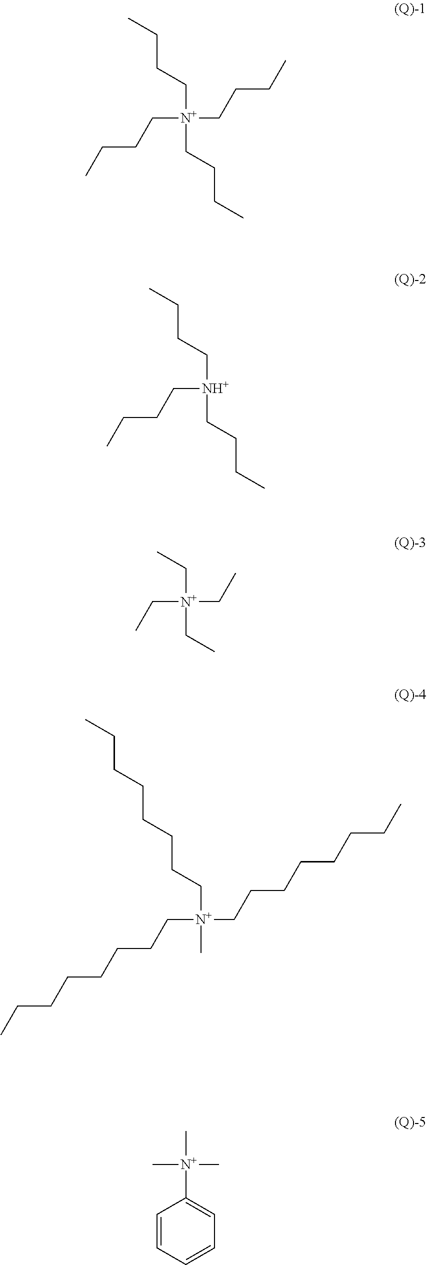

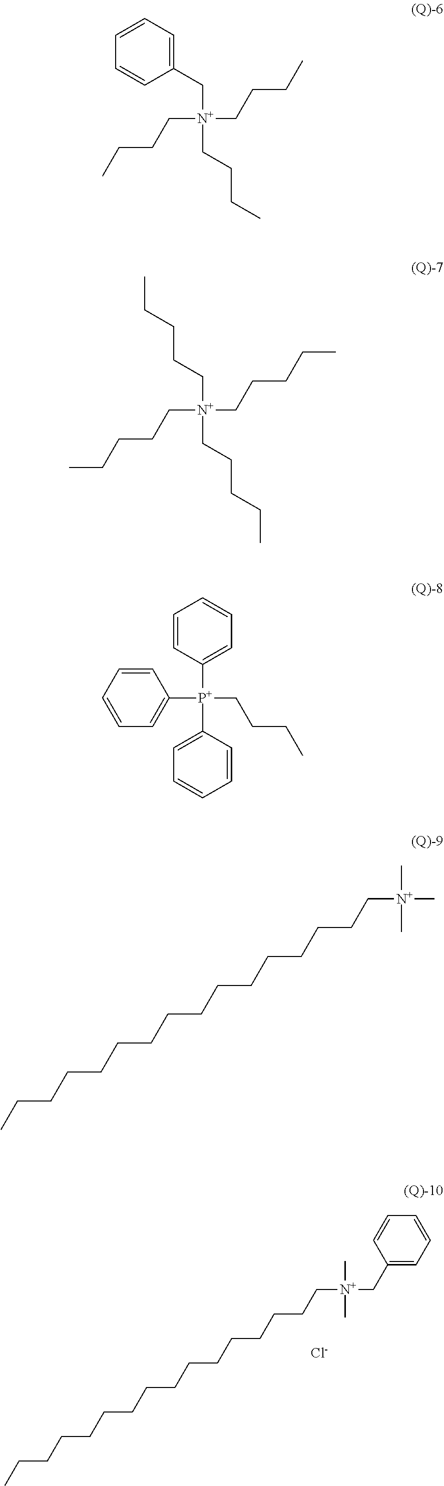

The various components used in the method for producing the actinic ray-sensitive or radiation-sensitive resin composition of Examples and Comparative Examples are shown below.

### <Onium Salt Compound (A)>

As the onium salt compound (A), the above-described compounds A1 to A26 were used. In Comparative Examples, the above-described compound RA1 was used. In Reference Examples, the above-described compound RA2 was used.

### <Resin (B)>

Resins B1 to B12 were used as the resin (B).

The structures of the resins B1 to B12 are shown in Tables 6 and 7 below. The content ratio (content with respect to all repeating units in the resin) of the following repeating unit is a molar ratio.

The weight-average molecular weight (Mw) and the dispersity (PDI=Mw/Mn) of the resin were measured by GPC (carrier: tetrahydrofuran (THF)) (values expressed in terms of polystyrene equivalent). In addition, the content of the repeating unit was measured by ¹³C-nuclear magnetic resonance (NMR).

TABLE 6

| | | Composition ratio | | |
|---|---|---|---|---|
| | Structure | (molar ratio) | Mw | PDI |
| B1 | 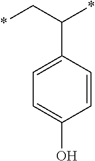 [SMILES: *CC(*)c1ccc(O)cc1] | 34/13/53 | 6500 | 1.60 |
| | 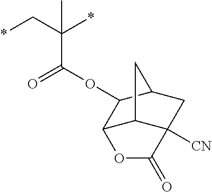 [SMILES: *CC(*)(C)C(=O)OC1C2CC3C1OC(=O)C3(C#N)C2] | | | |
| | 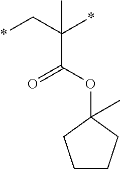 [SMILES: *CC(*)(C)C(=O)OC1(C)CCCC1] | | | |
| B2 | 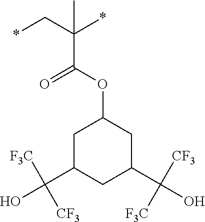 [SMILES: *CC(*)(C)C(=O)OC1CC(C(O)(C(F)(F)F)C(F)(F)F)CC(C(O)(C(F)(F)F)C(F)(F)F)C1] | 16/31/53 | 7200 | 1.58 |
| | 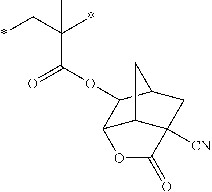 [SMILES: *CC(*)(C)C(=O)OC1C2CC3C1OC(=O)C3(C#N)C2] | | | |
| | 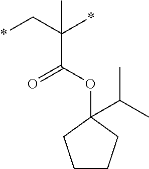 [SMILES: *CC(*)(C)C(=O)OC1(C(C)C)CCCC1] | | | |
| B3 | 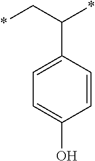 [SMILES: *CC(*)c1ccc(O)cc1] | 43/11/43/3 | 8000 | 1.62 |
| | 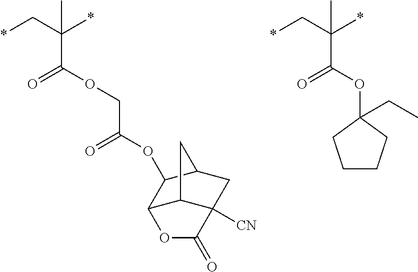 [SMILES: *CC(*)(C)C(=O)OCC(=O)OC1C2CC3C1OC(=O)C3(C#N)C2 ; *CC(*)(C)C(=O)OC1(CC)CCCC1] | | | |
| | 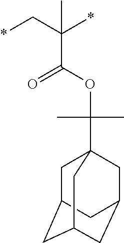 [SMILES: *CC(*)(C)C(=O)OC(C)(C)C12CC3CC(CC(C3)C1)C2] | | | |
| B4 | 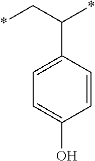 [SMILES: *CC(*)c1ccc(O)cc1] | 49/20/31 | 7800 | 1.71 |
| | 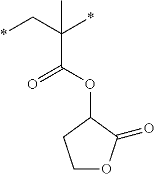 [SMILES: *CC(*)(C)C(=O)OC1CCOC1=O] | | | |
| | 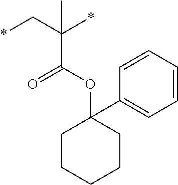 [SMILES: *CC(*)(C)C(=O)OC1(c2ccccc2)CCCCC1] | | | |
| B5 | 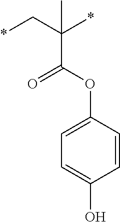 [SMILES: *CC(*)(C)C(=O)Oc1ccc(O)cc1] | 27/21/39/13 | 8400 | 1.78 |
| | 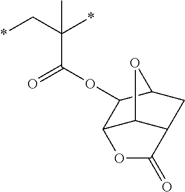 [SMILES: *CC(*)(C)C(=O)OC1C2CC3C(=O)OC1C3O2] | | | |
| | 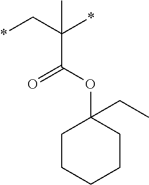 [SMILES: *CC(*)(C)C(=O)OC1(CC)CCCCC1] | | | |
| | 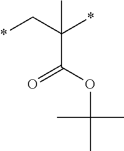 [SMILES: *CC(*)(C)C(=O)OC(C)(C)C] | | | |
| B6 | 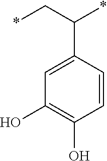 [SMILES: *CC(*)c1ccc(O)c(O)c1] | 36/7/57 | 12000 | 1.53 |
| | 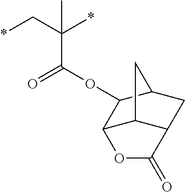 [SMILES: *CC(*)(C)C(=O)OC1C2CC3C(=O)OC1C3C2] | | | |
| | 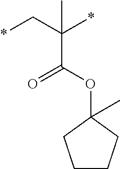 [SMILES: *CC(*)(C)C(=O)OC1(C)CCCC1] | | | |
| B7 | 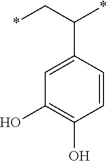 [SMILES: *CC(*)c1ccc(O)c(O)c1] | 31/7/3/59 | 8600 | 1.61 |
| | 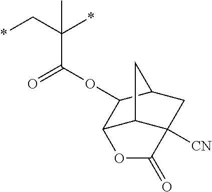 [SMILES: *CC(*)(C)C(=O)OC1C2CC3C1OC(=O)C3(C#N)C2] | | | |
| | 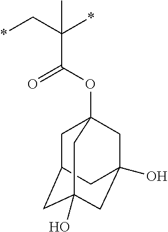 [SMILES: *CC(*)(C)C(=O)OC12CC3CC(O)(CC(O)(C3)C1)C2] | | | |
| | 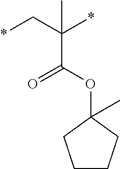 [SMILES: *CC(*)(C)C(=O)OC1(C)CCCC1] | | | |
| B8 | 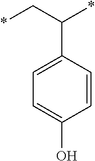 [SMILES: *CC(*)c1ccc(O)cc1] | 31/14/14/41 | 8000 | 1.58 |
| | 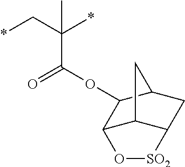 [SMILES: *CC(*)(C)C(=O)OC1C2CC3C1OS(=O)(=O)C3C2] | | | |
| | 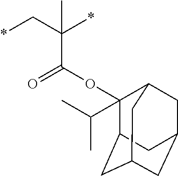 [SMILES: *CC(*)(C)C(=O)OC1(C(C)C)C2CC3CC(C2)CC1C3] | | | |
| | 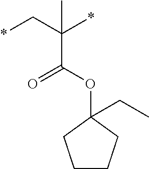 [SMILES: *CC(*)(C)C(=O)OC1(CC)CCCC1] | | | |

TABLE 7

| | | Composition ratio | | |
|---|---|---|---|---|
| | Structure | (molar ratio) | Mw | PDI |
| B9 | 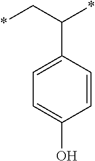 [SMILES: *CC(*)c1ccc(O)cc1] | 50/20/30 | 7500 | 1.51 |
| | 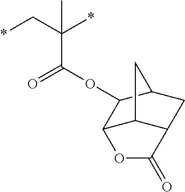 [SMILES: *CC(*)(C)C(=O)OC1C2CC3C(=O)OC1C3C2] | | | |
| | 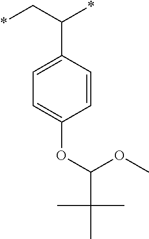 [SMILES: *CC(*)c1ccc(OC(OC)C(C)(C)C)cc1] | | | |
| B10 | 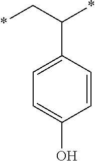 [SMILES: *CC(*)c1ccc(O)cc1] | 50/10/40 | 7000 | 1.58 |
| | 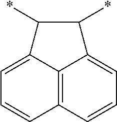 [SMILES: *C1c2cccc3cccc(c23)C1*] | | | |
| | 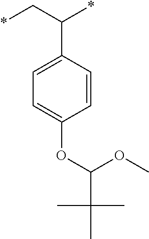 [SMILES: *CC(*)c1ccc(OC(OC)C(C)(C)C)cc1] | | | |
| B11 | 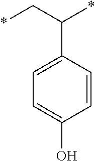 [SMILES: *CC(*)c1ccc(O)cc1] | 60/40 | 5000 | 1.52 |
| | 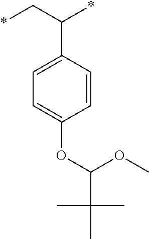 [SMILES: *CC(*)c1ccc(OC(OC)C(C)(C)C)cc1] | | | |
| B12 | 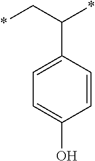 [SMILES: *CC(*)c1ccc(O)cc1] | 70/30 | 5500 | 1.55 |
| | 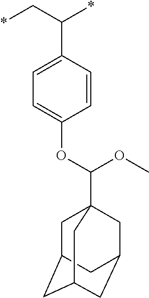 [SMILES: *CC(*)c1ccc(OC(OC)C23CC4CC(CC(C4)C2)C3)cc1] | | | |

### <Photoacid Generator (C)>

The photoacid generator C1 used is shown below.

The photoacid generator C1 was produced with reference to the method described in JP2013-160955A.

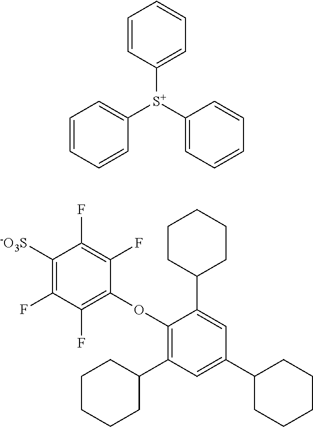

### <Acid Diffusion Control Agent (D)>

The acid diffusion control agent D1 used is shown below.

The acid diffusion control agent D1 was produced with reference to the method described in WO2020/175495A.

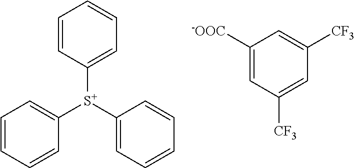

### <Hydrophobic Resin (E)>

The hydrophobic resins E1 and E2 used are shown below.

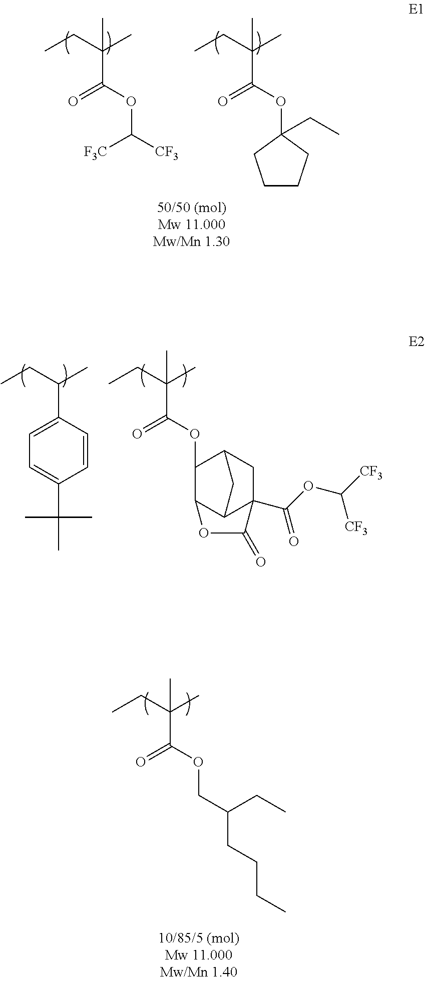

### <Solvent (F)>

The solvents F-1 to F-6 used are shown below.
F-1: Propylene glycol monomethyl ether acetate (PGMEA)
F-2: Propylene glycol monomethyl ether (PGME)
F-3: γ-Butyrolactone
F-4: Ethyl lactate
F-5: Cyclohexanone
F-6: 2-Heptanone

### Examples 1 to 49, Comparative Example 1, and Reference Example 1

### (Preparation of Actinic Ray-Sensitive or Radiation-Sensitive Resin Composition)

Each component shown in Tables 8 to 10 was dissolved in the solvent shown in Tables 8 to 10 and mixed so that the concentration of solid contents was 2.0% by mass. Next, the obtained mixed liquid was filtered initially through a polyethylene-made filter having a pore diameter of 50 nm, then through a nylon-made filter having a pore diameter of 10 nm, and lastly through a polyethylene-made filter having a pore diameter of 5 nm in this order to prepare an actinic ray-sensitive or radiation-sensitive resin composition (resist composition).

The content of each component in Tables 8 to 10 is a mass-based proportion with respect to the total solid content of each resist composition. “%” is based on a mass (that is, by mass”). The concentration of solid contents means a mass percentage of the mass of other components excluding the solvent with respect to the total mass of each resist composition.

The types of compounds used and the mass ratios thereof are shown in Tables 8 to 10 for the solvents.

TABLE 8

| | | Onium salt | | | | Photoacid | | | | Hydrophobic | | | |
|---|---|---|---|---|---|---|---|---|---|---|---|---|---|
| | | compound (A) | | Resin (B) | | generator (C) | | Acid diffusion control | | resin (E) | | | |
| | | | Content | | Content | | Content | agent (D) | | | Content | Solvent (F) | |
| | Com- | | (% by | | (% by | | (% by | | Content (% | | (% by | | Content ratio |
| | position | Type | mass) | Type | mass) | Type | mass) | Type | by mass) | Type | mass) | Type | (mass ratio) |
| Example 1 | R1 | A1 | 17.5 | B1 | 74.1 | C1 | 8.4 | — | — | — | — | F-1/F-2 | 80/20 |
| Example 2 | R2 | A1 | 17.5 | B2 | 74.1 | C1 | 8.4 | — | — | — | — | F-1/F-2 | 60/40 |
| Example 3 | R3 | A1 | 17.5 | B2 | 73.1 | C1 | 8.4 | — | — | E1 | 1.0 | F-1/F-2 | 60/40 |
| Example 4 | R4 | A1 | 17.5 | B7 | 74.1 | C1 | 8.4 | — | — | — | — | F-1/F-2/F-4 | 80/10/10 |
| Example 5 | R5 | A2 | 17.5 | B1 | 74.1 | C1 | 8.4 | — | — | — | — | F-1/F-2 | 80/20 |
| Example 6 | R6 | A3 | 6.8 | B1 | 80.6 | C1 | 12.6 | — | — | — | — | F-1/F-2 | 40/60 |
| Example 7 | R7 | A3 | 6.8 | B6 | 80.6 | C1 | 12.6 | — | — | — | — | F-1/F-2 | 60/40 |
| Example 8 | R8 | A4 | 17.5 | B1 | 74.1 | C1 | 8.4 | — | — | — | — | F-1/F-2/F-4 | 80/10/10 |
| Example 9 | R9 | A4 | 17.5 | B2 | 74.1 | C1 | 8.4 | — | — | — | — | F-1/F-2 | 70/30 |
| Example 10 | R10 | A4 | 17.5 | B7 | 74.1 | C1 | 8.4 | — | — | — | — | F-1/F-2/F-3 | 80/10/10 |
| Example 11 | R11 | A5 | 17.5 | B1 | 74.1 | C1 | 8.4 | — | — | — | — | F-1/F-5 | 90/10 |
| Example 12 | R12 | A6 | 17.3 | B1 | 78.4 | — | — | D1 | 4.3 | — | — | F-1/F-2 | 60/40 |
| Example 13 | R13 | A6 | 17.3 | B1 | 77.1 | — | — | D1 | 4.3 | E2 | 1.3 | F-1/F-2 | 60/40 |
| Example 14 | R14 | A6 | 17.3 | B2 | 78.4 | — | — | D1 | 4.3 | — | — | F-1/F-3/F-4 | 80/10/10 |
| Example 15 | R15 | A6 | 17.3 | B6 | 78.4 | — | — | D1 | 4.3 | — | — | F-1/F-3/F-4 | 80/15/5 |
| Example 16 | R16 | A7 | 25.6 | B1 | 73.1 | — | — | D1 | 1.3 | — | — | F-1/F-2/F-4 | 80/10/10 |
| Example 17 | R17 | A8 | 17.5 | B1 | 74.1 | C1 | 8.4 | — | — | — | — | F-1/F-2/F-3 | 80/10/10 |
| Example 18 | R18 | A8 | 17.5 | B3 | 74.1 | C1 | 8.4 | — | — | — | — | F-1/F-2 | 60/40 |
| Example 19 | R19 | A8 | 17.5 | B6 | 74.1 | C1 | 8.4 | — | — | — | — | F-1/F-2/F-6 | 80/10/10 |
| Example 20 | R20 | A9 | 17.3 | B1 | 78.4 | — | — | D1 | 4.3 | — | — | F-1/F-2 | 70/30 |
| Example 21 | R21 | A9 | 17.3 | B3 | 78.4 | — | — | D1 | 4.3 | — | — | F-1/F-3/F-4 | 80/15/5 |
| Example 22 | R22 | A9 | 17.3 | B7 | 78.4 | — | — | D1 | 4.3 | — | — | F-1/F-2 | 95/5 |
| Example 23 | R23 | A10 | 3.3 | B1 | 84.9 | C1 | 11.8 | — | — | — | — | F-1/F-3 | 85/15 |
| Example 24 | R24 | A10 | 3.3 | B5 | 84.9 | C1 | 11.8 | — | — | — | — | F-1/F-2 | 40/60 |
| Example 25 | R25 | A11 | 25.6 | B1 | 73.1 | — | — | D1 | 1.3 | — | — | F-1/F-2/F-6/F-3 | 85/7/7/1 |

TABLE 9

| | | Onium salt | | | | Photoacid | | Acid diffusion | | Hydrophobic | | | |
|---|---|---|---|---|---|---|---|---|---|---|---|---|---|
| | | compound (A) | | Resin (B) | | generator (C) | | control agent (D) | | resin (E) | | | |
| | | | Content | | Content | | Content | | Content | | Content | Solvent (F) | |
| | | | (% by | | (% by | | (% by | | (% by | | (% by | | Content ratio |
| | Composition | Type | mass) | Type | mass) | Type | mass) | Type | mass) | Type | mass) | Type | (mass ratio) |
| Example 26 | R26 | A11 | 25.6 | B6 | 73.1 | — | — | D1 | 1.3 | — | — | F-1/F-2/F-3 | 75/15/10 |
| Example 27 | R27 | A12 | 6.8 | B1 | 80.6 | C1 | 12.6 | — | — | — | — | F-1/F-2/F-4 | 80/15/5 |
| Example 28 | R28 | A12 | 6.8 | B4 | 80.6 | C1 | 12.6 | — | — | — | — | F-1/F-3 | 85/15 |
| Example 29 | R29 | A13 | 17.3 | B1 | 78.4 | — | — | D1 | 4.3 | — | — | F-1/F-2/F-4 | 80/10/10 |
| Example 30 | R30 | A13 | 17.3 | B8 | 78.4 | — | — | D1 | 4.3 | — | — | F-1/F-2 | 70/30 |
| Example 31 | R31 | A14 | 17.3 | B1 | 78.4 | — | — | D1 | 4.3 | — | — | F-1/F-2 | 70/30 |
| Example 32 | R32 | A14 | 17.3 | B2 | 78.4 | — | — | D1 | 4.3 | — | — | F-1/F-2 | 80/20 |
| Example 33 | R33 | A15 | 17.3 | B1 | 78.4 | — | — | D1 | 4.3 | — | — | F-1/F-3 | 85/15 |
| Example 34 | R34 | A16 | 15.6 | B1 | 81.3 | — | — | D1 | 3.1 | — | — | F-1/F-2 | 60/40 |
| Example 35 | R35 | A16 | 15.6 | B4 | 81.3 | — | — | D1 | 3.1 | — | — | F-1/F-2/F-3 | 80/10/10 |
| Example 36 | R36 | A17 | 15.6 | B1 | 81.3 | — | — | D1 | 3.1 | — | — | F-1/F-2 | 70/30 |
| Example 37 | R37 | A17 | 15.6 | B8 | 81.3 | — | — | D1 | 3.1 | — | — | F-1/F-2/F-6 | 80/15/5 |
| Example 38 | R38 | A18 | 17.3 | B1 | 78.4 | — | — | D1 | 4.3 | — | — | F-1/F-2/F-5 | 75/20/5 |
| Example 39 | R39 | A19 | 17.3 | B1 | 78.4 | — | — | D1 | 4.3 | — | — | F-1/F-4 | 90/10 |
| Example 40 | R40 | A20 | 20.2 | B1 | 74.9 | C1 | 4.9 | — | — | — | — | F-1/F-2 | 70/30 |
| Example 41 | R41 | A20 | 20.2 | B5 | 74.9 | C1 | 4.9 | — | — | — | — | F-1/F-2 | 50/50 |
| Example 42 | R42 | A21 | 6.8 | B1 | 80.6 | C1 | 12.6 | — | — | — | — | F-1/F-2 | 80/20 |
| Example 43 | R43 | A21 | 6.8 | B4 | 80.6 | C1 | 12.6 | — | — | — | — | F-1/F-2/F-6 | 80/10/10 |
| Example 44 | R44 | A1/A9 | 17.5/8.4 | B6 | 74.1 | — | — | — | — | — | — | F-1/F-2/F-4 | 80/10/10 |
| Example 45 | R45 | A22 | 17.5 | B1 | 74.1 | C1 | 8.4 | — | — | — | — | F-1/F-2 | 80/20 |
| Comparative | RR1 | RA1 | 17.5 | B1 | 74.1 | C1 | 8.4 | — | — | — | — | F-1/F-2/F-4 | 80/10/10 |
| Example 1 | | | | | | | | | | | | | |
| Reference | RR2 | RA2 | 17.5 | B1 | 74.1 | C1 | 8.4 | — | — | — | — | F-1/F-2 | 80/20 |
| Example 1 | | | | | | | | | | | | | |

TABLE 10

| | | | | | | | | Acid | | | | | |
|---|---|---|---|---|---|---|---|---|---|---|---|---|---|
| | | | | | | | | diffusion | | | | | |
| | | Onium salt | | | | Photoacid | | control agent | | Hydrophobic | | Solvent (F) | |
| | | compound (A) | | Resin (B) | | generator (C) | | (D) | | resin (E) | | | Content |
| | | | Content | | Content | | Content | | Content | | Content | | ratio |
| | | | (% by | | (% by | | (% by | | (% by | | (% by | | (mass |
| | Composition | Type | mass) | Type | mass) | Type | mass) | Type | mass) | Type | mass) | Type | ratio) |
| Example | R46 | A23/A25 | 12.5/10.4 | B9 | 77.1 | — | — | — | — | — | — | F-1/F-2/F-3 | 80/10/10 |
| 46 | | | | | | | | | | | | | |
| Example | R47 | A23/A26 | 15.2/8.2 | B10 | 76.6 | — | — | — | — | — | — | F-1/F-4 | 60/40 |
| 47 | | | | | | | | | | | | | |
| Example | R48 | A24/A26 | 18.3/12.1 | B11 | 69.6 | — | — | — | — | — | — | F-1/F-2/F-3 | 80/10/10 |
| 48 | | | | | | | | | | | | | |
| Example | R49 | A24/A26 | 11.1/10.6 | B12 | 78.3 | — | — | — | — | — | — | F-1/F-2/F-3 | 80/10/10 |
| 49 | | | | | | | | | | | | | |

### [Pattern Formation 1: EUV Exposure and Aqueous Alkaline Solution Development]

A composition for forming an underlayer film, AL412 (manufactured by Brewer Science, Inc.), was applied to a silicon wafer and baked at 205° C. for 60 seconds to form an underlying film having a film thickness of 20 nm. Resist compositions shown in Tables 8 to 10 were applied on the underlying film and baked at 100° C. for 60 seconds to form a resist film having a film thickness of 30 nm.

The silicon wafer having the obtained resist film was subjected to a pattern irradiation using an EUV exposure device (manufactured by Exitech Ltd., Micro Exposure Tool, NA 0.3, Quadrupole, outer sigma 0.68, inner sigma 0.36). As a reticle, a mask having a line size=25 nm and a line:space=1:1 was used.

The resist film after the exposure was baked at 90° C. for 60 seconds, developed with a tetramethylammonium hydroxide aqueous solution (2.38% by mass) for 30 seconds, and then rinsed with pure water for 30 seconds. Thereafter, the resist film was spin-dried to obtain a positive tone pattern.

### <Evaluation of Line Width Roughness (LWR) Performance>

In a case of observing a line-and-space pattern of 25 nm (1:1) resolved at an optimum exposure amount in a case of resolving a line pattern having an average line width of 25 nm from the upper part of the pattern using a scanning electron microscope (SEM (S-9380II, manufactured by Hitachi, Ltd.)), the line width was observed at 250 locations, and the standard deviation (a) thereof was obtained. The measurement variation of the line width was evaluated by 36, and the value of 36 was defined as LWR (nm). As the value is smaller, the performance is better.

The LWR is preferably 4.0 nm or less, more preferably 3.5 nm or less, and particularly preferably 3.2 nm or less.

The resist compositions and the results used in Tables 11 and 12 are shown.

TABLE 11

| Example | Composition | LWR (nm) |
|---|---|---|
| Example P1 | R1 | 2.9 |
| Example P2 | R2 | 2.9 |
| Example P3 | R3 | 2.8 |
| Example P5 | R5 | 3.4 |
| Example P6 | R6 | 2.9 |
| Example P7 | R7 | 3.0 |
| Example P8 | R8 | 2.8 |
| Example P9 | R9 | 2.8 |
| Example P10 | R10 | 2.9 |
| Example P11 | R11 | 3.3 |
| Example P12 | R12 | 3.0 |
| Example P13 | R13 | 2.9 |
| Example P14 | R14 | 2.9 |
| Example P16 | R16 | 2.9 |
| Example P17 | R17 | 3.0 |
| Example P18 | R18 | 3.1 |
| Example P20 | R20 | 2.9 |
| Example P21 | R21 | 2.9 |
| Example P22 | R22 | 2.9 |
| Example P23 | R23 | 2.9 |
| Example P25 | R25 | 2.8 |
| Example P27 | R27 | 3.1 |
| Example P28 | R28 | 3.1 |
| Example P29 | R29 | 3.1 |
| Example P31 | R31 | 3.5 |
| Example P32 | R32 | 3.5 |
| Example P33 | R33 | 3.8 |
| Example P34 | R34 | 3.1 |
| Example P35 | R35 | 3.1 |
| Example P36 | R36 | 3.0 |
| Example P38 | R38 | 3.4 |
| Example P39 | R39 | 3.6 |
| Example P40 | R40 | 2.8 |
| Example P41 | R41 | 2.9 |
| Example P42 | R42 | 3.9 |
| Example P44 | R44 | 2.9 |
| Example P45 | R45 | 3.2 |
| Comparative | RR1 | 4.2 |
| Example PR1 | | |
| Reference | RR2 | 4.2 |
| Example PR2 | | |

TABLE 12

| Example | Composition | LWR (nm) |
|---|---|---|
| Example P46 | R46 | 2.9 |
| Example P47 | R47 | 3.1 |
| Example P48 | R48 | 2.8 |
| Example P49 | R49 | 3.0 |

### [Pattern Formation 2: EUV Exposure and Organic Solvent Development]

A composition for forming an underlayer film, AL412 (manufactured by Brewer Science, Inc.), was applied to a silicon wafer and baked at 205° C. for 60 seconds to form an underlying film having a film thickness of 20 nm. Resist compositions shown in Tables 8 and 9 were applied on the underlying film and baked at 100° C. for 60 seconds to form a resist film having a film thickness of 30 nm.

The silicon wafer having the obtained resist film was subjected to a pattern irradiation using an EUV exposure device (manufactured by Exitech Ltd., Micro Exposure Tool, NA 0.3, Quadrupole, outer sigma 0.68, inner sigma 0.36). As a reticle, a mask having a line size=25 nm and a line:space=1:1 was used.

The resist film after the exposure was baked at 90° C. for 60 seconds, developed with n-butyl acetate for 30 seconds, and spin-dried to obtain a negative tone pattern.

### <Evaluation of LWR Performance>

The LWR performance was evaluated by the same method as described above.

The resist compositions and the results used in Table 13 are shown.

TABLE 13

| Example | Composition | LWR (nm) |
|---|---|---|
| Example N1 | R1 | 3.0 |
| Example N2 | R2 | 3.0 |
| Example N3 | R3 | 2.9 |
| Example N4 | R4 | 2.9 |
| Example N5 | R5 | 3.5 |
| Example N6 | R6 | 3.0 |
| Example N7 | R7 | 3.1 |
| Example N8 | R8 | 2.9 |
| Example N10 | R10 | 3.0 |
| Example N11 | R11 | 3.4 |
| Example N12 | R12 | 3.1 |
| Example N13 | R13 | 3.0 |
| Example N15 | R15 | 3.0 |
| Example N16 | R16 | 3.0 |
| Example N17 | R17 | 3.1 |
| Example N19 | R19 | 3.0 |
| Example N20 | R20 | 3.0 |
| Example N22 | R22 | 3.0 |
| Example N23 | R23 | 3.0 |
| Example N24 | R24 | 3.0 |
| Example N25 | R25 | 2.9 |
| Example N26 | R26 | 2.9 |
| Example N27 | R27 | 3.2 |
| Example N28 | R28 | 3.2 |
| Example N29 | R29 | 3.2 |
| Example N30 | R30 | 3.2 |
| Example N31 | R31 | 3.6 |
| Example N32 | R33 | 3.9 |
| Example N33 | R34 | 3.2 |
| Example N34 | R35 | 3.2 |
| Example N35 | R36 | 3.1 |
| Example N36 | R37 | 3.2 |
| Example N37 | R38 | 3.5 |
| Example N38 | R39 | 3.7 |
| Example N40 | R40 | 2.9 |
| Example N41 | R41 | 3.0 |
| Example N42 | R42 | 4.0 |
| Example N43 | R43 | 4.0 |
| Example N44 | R44 | 3.0 |
| Example N45 | R45 | 3.3 |
| Comparative | RR1 | 4.3 |
| Example NR1 | | |
| Reference | RR2 | 4.3 |
| Example NR2 | | |

### [Pattern Formation 3: Electron Beams (EB) Exposure and Aqueous Alkaline Solution Development]

The resist compositions shown in Tables 8 to 10 were uniformly applied onto a hexamethyldisilazane-treated silicon substrate using a spin coater. Then, the composition was heated and dried at 120° C. for 90 seconds on a hot plate to form a resist film with a film thickness of 35 nm.

The obtained resist film was irradiated with electron beams through a 6% halftone mask with a line width of 24 nm and a 1:1 line-and-space pattern, using an electron beam irradiation device (HL750 manufactured by Hitachi, Ltd., accelerating voltage of 50 keV). Immediately after irradiation, the resist film was heated on a hot plate at 110° C. for 60 seconds. The resist film was further developed at 23° C. for 60 seconds using a 2.38%-by-mass aqueous tetramethylammonium hydroxide (TMAH) solution, rinsed with pure water for 30 seconds, and then spin-dried to obtain a positive tone pattern.

### <Evaluation of LWR Performance>

The LWR performance was evaluated by the same method as described above.

The resist compositions and the results used in Tables 14 and 15 are shown.

TABLE 14

| Example | Composition | LWR (nm) |
|---|---|---|
| Example EP1 | R1 | 3.1 |
| Example EP2 | R2 | 3.1 |
| Example EP3 | R3 | 3.0 |
| Example EP5 | RS | 3.6 |
| Example EP6 | R6 | 3.1 |
| Example EP8 | R8 | 3.0 |
| Example EP9 | R9 | 3.0 |
| Example EP10 | R10 | 3.1 |
| Example EP11 | R11 | 3.5 |
| Example EP12 | R12 | 3.2 |
| Example EP13 | R13 | 3.1 |
| Example EP16 | R16 | 3.1 |
| Example EP18 | R18 | 3.3 |
| Example EP20 | R20 | 3.1 |
| Example EP23 | R23 | 3.1 |
| Example EP25 | R25 | 3.0 |
| Example EP27 | R27 | 3.3 |
| Example EP29 | R29 | 3.3 |
| Example EP32 | R32 | 3.7 |
| Example EP35 | R35 | 3.3 |
| Example EP39 | R39 | 3.8 |
| Example EP42 | R42 | 4.1 |
| Example EP44 | R44 | 3.1 |
| Example EP45 | R45 | 3.4 |
| Comparative | RR1 | 4.4 |
| Example ER1 | | |
| Reference | RR2 | 4.4 |
| Example ER2 | | |

TABLE 15

| Example | Composition | LWR (nm) |
|---|---|---|
| Example EP46 | R46 | 3.0 |
| Example EP47 | R47 | 3.1 |
| Example EP48 | R48 | 3.0 |
| Example EP49 | R49 | 3.1 |

The exposure with the electron beam irradiation device (HL750 manufactured by Hitachi, Ltd.) is a single-beam type, and is not a multi-beam type in which a plurality of single beams are simultaneously scanned. However, the effect of replacing the multi-beam type with the single-beam type is only on the total drawing time, and it is assumed that the evaluation results of the obtained resolution and the LWR are equivalent to the evaluation results in a case where the multi-beam type is used.

From the results of Tables 11 to 15, it was found that the resist compositions used in Examples had excellent LWR performance.

According to the present invention, it is possible to provide a method for producing an actinic ray-sensitive or radiation-sensitive resin composition having excellent LWR performance, a pattern forming method including the method for producing, and a method for manufacturing an electronic device.

In addition, according to the present invention, it is possible to provide a method for producing an onium salt compound, which can be suitably used for the actinic ray-sensitive or radiation-sensitive resin composition, and an onium salt composition.

The present invention has been described in detail with reference to specific embodiments. To those skilled in the art, it is obvious that various changes or modifications can be added without departing from the gist and scope of the present invention.

What is claimed is:

1. A method for producing an actinic ray-sensitive or radiation-sensitive resin composition, the method comprising:
mixing an onium salt compound (1) represented by Formula (1A) or Formula (1B) with a salt compound (2) represented by Formula (2) in a non-aqueous solvent (S) to obtain an onium salt compound (3) represented by Formula (3A) or Formula (3B);
obtaining an onium salt compound (A) represented by Formula (4A) or Formula (4B) from the onium salt compound (3); and
mixing the onium salt compound (A) with a resin (B) whose solubility in a developer changes due to action of acid, 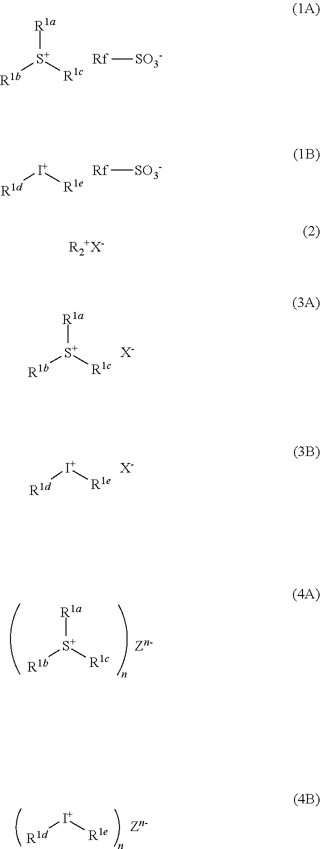
in Formulae (1A), (3A), and (4A), R^{1a}, R^{1b}, and R^{1c} each independently represent an alkyl group, a cycloalkyl group, an aryl group, or *—W₁—C(═O)Ar¹, two of R^{1a}, R^{1b}, and R^{1c} may be bonded to each other to form a ring, and *represents a linkage site to S⁺, W₁ represents a single bond or an alkylene group, and Ar¹ represents an aryl group,
in Formulae (1B), (3B), and (4B), R^{1d} and R^{1e} each independently represent an alkyl group, a cycloalkyl group, or an aryl group,
in Formulae (1A) and (1B), Rf represents an alkyl group or an aryl group which includes one or more fluorine atoms,
in Formula (2), R₂⁺ represents an organic cation which does not have a polymer structure, and X⁻ represents Cl⁻ or Br⁻,
in Formulae (3A) and (3B), X⁻ represents Cl⁻ or Br⁻,
in Formulae (4A) and (4B), Zⁿ⁻ represents an n-valent organic anion, and n represents an integer of 1 or more.

2. The method for producing an actinic ray-sensitive or radiation-sensitive resin composition according to claim 1,
wherein R^{1a}, R^{1b}, and R^{1c} in Formula (1A), Formula (3A), and Formula (4A) each independently represent an alkyl group which does not include a halogen atom, a cycloalkyl group, an aryl group, or *—W₁—C(═O)Ar¹,
where two of R^{1a}, R^{1b}, and R^{1c} may be bonded to each other to form a ring, and *represents a linkage site to S⁺, W₁ represents a single bond or an alkylene group, and Ar¹ represents an aryl group.

3. The method for producing an actinic ray-sensitive or radiation-sensitive resin composition according to claim 1,
wherein R₂⁺ in Formula (2) is an organic cation represented by Formula (2B), 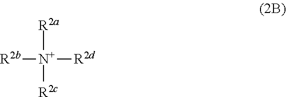
in Formula (2B), R^{2a} to R^{2d} each independently represent an alkyl group, a cycloalkyl group, or an aryl group.

4. The method for producing an actinic ray-sensitive or radiation-sensitive resin composition according to claim 1,
wherein the non-aqueous solvent (S) contains at least one of an ester-based solvent or an ether-based solvent.

5. The method for producing an actinic ray-sensitive or radiation-sensitive resin composition according to claim 1,
wherein R^{1a}, R^{1b}, and R^{1c} in Formulae (1A), (3A), and (4A) are aryl groups,
where two of R^{1a}, R^{1b}, and R^{1c} may be bonded to each other to form a ring.

6. A method for producing an onium salt compound (A) for an actinic ray-sensitive or radiation-sensitive resin composition, the method comprising:
mixing an onium salt compound (1) represented by Formula (1A) or Formula (1B) with a salt compound (2) represented by Formula (2) in a non-aqueous solvent (S) to obtain an onium salt compound (3) represented by Formula (3A) or Formula (3B); and
obtaining an onium salt compound (A) represented by Formula (4A) or Formula (4B) from the onium salt compound (3), 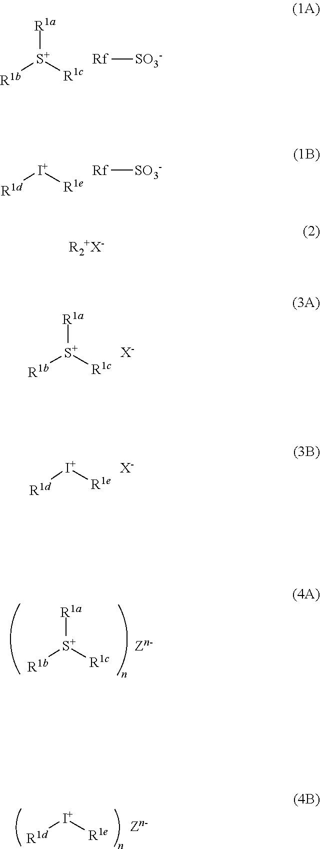
in Formulae (1A), (3A), and (4A), R^{1a}, R^{1b}, and R^{1c} each independently represent an alkyl group, a cycloalkyl group, an aryl group, or *—W₁—C(═O)Ar¹, two of R^{1a}, R^{1b}, and R^{1c} may be bonded to each other to form a ring, and *represents a linkage site to S⁺, W₁ represents a single bond or an alkylene group, and Ar represents an aryl group,
in Formulae (1B), (3B), and (4B), R^{1d} and R^{1e} each independently represent an alkyl group, a cycloalkyl group, or an aryl group,
in Formulae (1A) and (1B), Rf represents an alkyl group or an aryl group which includes one or more fluorine atoms,
in Formula (2), R₂⁺ represents an organic cation which does not have a polymer structure, and X⁻ represents Cl⁻ or Br⁻,
in Formulae (3A) and (3B), X⁻ represents Cl⁻ or Br⁻,
in Formulae (4A) and (4B), Zⁿ⁻ represents an n-valent organic anion, and n represents an integer of 1 or more.

7. The method for producing an onium salt compound (A) according to claim 6,
wherein R^{1a}, R^{1b}, and R^{1c} in Formula (1A), Formula (3A), and Formula (4A) each independently represent an alkyl group which does not include a halogen atom, a cycloalkyl group, an aryl group, or *—W₁—C(═O)Ar¹,
where two of R^{1a}, R^{1b}, and R^{1c} may be bonded to each other to form a ring, and *represents a linkage site to S⁺, W₁ represents a single bond or an alkylene group, and Ar¹ represents an aryl group.

8. The method for producing an onium salt compound (A) according to claim 6,
wherein R₂⁺ in Formula (2) is an organic cation represented by Formula (2B), 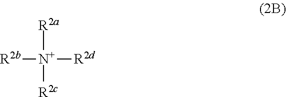
in Formula (2B), R^{2a} to R^{2d} each independently represent an alkyl group, a cycloalkyl group, or an aryl group.

9. The method for producing an onium salt compound (A) according to claim 6,
wherein the non-aqueous solvent (S) contains at least one of an ester-based solvent or an ether-based solvent.

10. The method for producing an onium salt compound (A) according to claim 6,
wherein R^{1a}, R^{1b}, and R^{1c} in Formulae (1A), (3A), and (4A) are aryl groups,
where two of R^{1a}, R^{1b}, and R^{1c} may be bonded to each other to form a ring.

11. An onium salt composition including an onium salt compound, comprising:
0.001 mol % to 3 mol % of an organic cation represented by Formula (2A) with respect to 1 mol of the onium salt compound represented by Formula (4A) or Formula (4B), 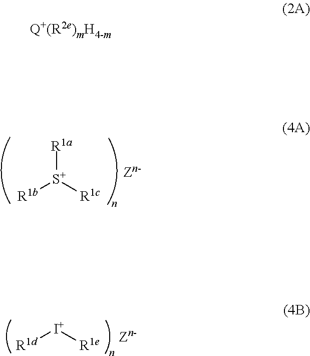
in Formula (2A), Q represents an N atom or a P atom, m represents an integer of 1 to 4, R^{2e} represents an alkyl group, a cycloalkyl group, or an aryl group, and a plurality of R^{2e}'s may be the same as or different from each other, and R^{2e}'s adjacent to each other may form a ring,
in Formula (4A), R^{1a}, R^{1b}, and R^{1c} each independently represent an alkyl group, a cycloalkyl group, an aryl group, or *—W₁—C(═O)Ar¹, two of R^{1a}, R^{1b}, and R^{1c} may be bonded to each other to form a ring, and *represents a linkage site to S⁺, W₁ represents a single bond or an alkylene group, and Ar¹ represents an aryl group,
in Formula (4B), R^{1d} and R^{1e} each independently represent an alkyl group, a cycloalkyl group, or an aryl group,
in Formulae (4A) and (4B), Zⁿ⁻ represents an n-valent organic anion, and n represents an integer of 1 or more.

12. The onium salt composition according to claim 11,
wherein the organic cation represented by Formula (2A) is represented by Formula (2B), 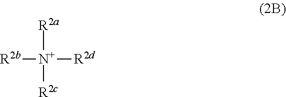
in Formula (2B), R^{2a} to R^{2d} each independently represent an alkyl group, a cycloalkyl group, or an aryl group.

13. The onium salt composition according to claim 11,
wherein R^{1a}, R^{1b}, and R^{1c} in Formula (4A) are aryl groups.

14. A pattern forming method comprising:
obtaining an actinic ray-sensitive or radiation-sensitive resin composition by the method for producing an actinic ray-sensitive or radiation-sensitive resin composition according to claim 1;
forming an actinic ray-sensitive or radiation-sensitive film on a substrate using the actinic ray-sensitive or radiation-sensitive resin composition;
exposing the actinic ray-sensitive or radiation-sensitive film; and
developing the exposed actinic ray-sensitive or radiation-sensitive film using a developer to form a pattern.

15. A method for manufacturing an electronic device, comprising:
the pattern forming method according to claim 14.
